(19) United States
(12) Patent Application Publication
(10) Pub. No.: US 2015/0329868 A1
(43) Pub. Date: Nov. 19, 2015

(54) MODIFIED PHOTOSYNTHETIC MICROORGANISMS FOR CONTINUOUS PRODUCTION OF CARBON-CONTAINING COMPOUNDS

(71) Applicant: Matrix Genetics, LLC, Seattle, WA (US)

(72) Inventors: Jason W. Hickman, San Diego, CA (US); Cameron Miller, Kirkland, WA (US); Mark Budde, Arcadia, CA (US); James Roberts, Seattle, WA (US); Fred Cross, New York, NY (US); Kimberly Marie Kotovic, Seattle, WA (US); Paul Warrener, Gaithersburg, MD (US); Brett K. Kaiser, Seattle, WA (US); Michael Carleton, Kirkland, WA (US)

(21) Appl. No.: 14/376,387

(22) PCT Filed: Jan. 31, 2013
(86) PCT No.: PCT/US13/24142

§ 371 Date: Aug. 1, 2014

Related U.S. Application Data
(60) Provisional application No. 61/595,017, filed on Feb. 3, 2012.
(60) Provisional application No. 61/625,887, filed on Apr. 18, 2012.
(60) Provisional application No. 61/753,339, filed on Jan. 16, 2013.

(51) Int. Cl. C12N 15/74; C12P 7/46; C12P 7/04; C12P 19/02; C12P 13/00; C12N 9/10; C12N 9/12; C12N 9/90; C12P 9/00; C12P 7/50; C12P 7/48

(52) U.S. Cl. CPC C12N 15/74; C12P 7/50; C12P 7/46; C12P 7/04; C12P 19/02; C12P 7/48; C12N 9/1051; C12N 9/1241; C12N 9/90; C12P 9/00; C12P 13/001

(57) ABSTRACT

The present invention relates to a continuous production system for producing carbon-containing compounds, comprising a genetically modified photosynthetic microorganism, such as a Cyanobacterium, that contains one or more mutations or deletions in a glycogen biosynthesis or storage pathway. The system and methods provided herein facilitate the production of carbon-containing compounds under reduced growth conditions, including biofuels, lipids, and other specialty chemicals, such as fatty acids, alkanes, alkenes, wax esters, and triglycerides.

### CROSS-REFERENCE TO RELATED APPLICATION

This application claims the benefit under 35 U.S.C. §119(e) of U.S. Provisional Patent Application No. 61/595,017 filed Feb. 3, 2012, U.S. Provisional Patent Application No. 61/625,887 filed Apr. 18, 2012, and U.S. Provisional Patent Application No. 61/753,339 filed Jan. 16, 2013, where these Provisional Applications are incorporated herein by reference in its entirety.

### SEQUENCE LISTING

The Sequence Listing associated with this application is provided in text format in lieu of a paper copy, and is hereby incorporated by reference into the specification. The name of the text file containing the Sequence Listing is M077-0008PCT_ST25.txt. The text file is about 692 KB, was created on Jan. 16, 2013, and is being submitted electronically via EFS-Web.

### BACKGROUND

1. Technical Field

The present invention relates generally to a continuous production platform that utilizes photosynthetic microorganisms, e.g., Cyanobacteria, modified to uncouple photosynthesis from growth, to produce carbon-containing compounds, such as lipids, biofuels and other specialty chemicals.

2. Description of the Related Art

Fatty acids are carboxylic acids with an unbranched aliphatic tail or chain, the latter ranging from about four to about 28 carbon atoms in length. Triglycerides are neutral polar molecules consisting of glycerol esterified with three fatty acid molecules. Triglycerides and fatty acids can be utilized as carbon and energy storage molecules by most eukaryotic organisms, including plants and algae, and by certain prokaryotic organisms, including certain species of actinomycetes and members of the genus Acinetobacter.

Triglycerides and fatty acids may also be utilized as a feedstock in the production of biofuels and/or other various specialty chemicals. For example, triglycerides and free fatty acids may be subject to a transesterification reaction, in which an alcohol reacts with triglyceride oils or fatty acid molecules, such as those contained in vegetable oils, animal fats, recycled greases, to produce biodiesels such as fatty acid alkyl esters. When triglycerides are included in the starting material, such reactions also produce glycerin as a by-product, which can be purified for use in the pharmaceutical and cosmetic industries

Certain organisms can be utilized as a source of triglycerides or free fatty acids in the production of biofuels. For example, algae naturally produce triglycerides as energy storage molecules, and certain biofuel-related technologies are presently focused on the use of algae as a feedstock for biofuels. Algae are photosynthetic organisms, and the use of triglyceride-producing organisms such as algae provides the ability to produce biodiesel from sunlight, water, CO₂, macronutrients, and micronutrients. Algae, however, cannot be readily genetically manipulated, and produce much less oil (i.e., triglycerides, fatty acids) under culture conditions than in the wild.

Like algae, Cyanobacteria obtain energy from photosynthesis, utilizing chlorophyll A and water to reduce CO₂. Certain Cyanobacteria can produce metabolites, such as carbohydrates, proteins, and fatty acids, from just sunlight, water, CO₂, water, and inorganic salts. Unlike algae, Cyanobacteria can be genetically manipulated. For example, Synechococcus is a genetically manipulable, oligotrophic Cyanobacterium that thrives in low nutrient level conditions, and in the wild accumulates fatty acids in the form of lipid membranes to about 10% by dry weight. Cyanobacteria such as Synechococcus, however, produce no triglyceride energy storage molecules, since Cyanobacteria typically lack the essential enzymes involved in triglyceride synthesis. Instead, Synechococcus in the wild typically accumulates glycogen as its primary carbon storage form.

Clearly, therefore, there is a need in the art for modified photosynthetic microorganisms, including Cyanobacteria, capable of producing lipids such as triglycerides and fatty acids, e.g., to be used as feed stock in the production of biofuels.

### BRIEF SUMMARY

The present invention provides systems and methods for the production of carbon-containing compounds.

In one embodiment, the present invention includes a system for producing carbon-containing compounds, comprising: a modified photosynthetic organism that accumulates a reduced amount of glycogen as compared to the wild-type photosynthetic microorganism; and a culture system for culturing said modified photosynthetic organism under a stress condition, wherein said modified photosynthetic organism maintains photosynthetic activity and accumulates reduced biomass when grown under said stress condition as compared to when grown under non-stress conditions. In particular embodiments, said modified photosynthetic microorganism produces or accumulates intracellularly and/or secretes an increased amount of one or more carbon-containing compounds when grown under said stress condition as compared to when grown under non-stress conditions.

In particular embodiments of the systems and related methods of the present invention, said modified photosynthetic microorganism: has reduced expression of one or more genes of a glycogen biosynthesis or storage pathway as compared to the wild-type photosynthetic microorganism; and/or comprises one or more introduced polynucleotides encoding a protein that increases glycogen breakdown or secretion. In particular embodiments, said stress condition is a reduced level of an essential nutrient, which is optionally selected from nitrogen, sulfur, or phosphorous. In certain embodiments, said modified photosynthetic organism produces and/or secretes an increased amount of at least one carbon-containing compound under said stress condition as compared to the wild-type organism under said stress condition, or as compared to the same modified photosynthetic organism under a non-stress condition, for example, an essential nutrient replete condition.

In a related embodiment, the invention includes methods for producing a carbon-containing compound other than glycogen, comprising culturing in a culture media under a stress condition a modified photosynthetic organism that accumulates a reduced amount of glycogen as compared to the wild-type photosynthetic organism, wherein said modified photosynthetic organism maintains photosynthetic activity and accumulates reduced biomass when grown under said stress condition as compared to when grown under non-stress conditions. In particular embodiments, said modified photosynthetic organism produces and/or secretes an increased amount of one or more carbon-containing compounds when grown under said stress condition as compared to when grown under a non-stress condition, such as an essential nutrient replete condition. In certain embodiments, said modified photosynthetic microorganism: has reduced expression of one or more genes of a glycogen biosynthesis or storage pathway as compared to the wild-type photosynthetic microorganism; and/or comprises one or more introduced polynucleotides encoding a protein that increases glycogen breakdown or increases secretion of a glycogen precursor. In certain embodiments, the method further comprises harvesting said culture media after said modified photosynthetic organism has been cultured under said stress condition. In other related embodiments, the method comprises obtaining said carbon-containing compound from said harvested culture media. In another embodiment, the method further comprises harvesting said modified photosynthetic organism after it has been cultured under said stress condition. In a related embodiment, the method further comprises obtaining said carbon-containing compound from said harvested modified photosynthetic organism.

In particular embodiments of systems and methods of the present invention, said stress condition is a reduced level of an essential nutrient, which is optionally selected from nitrogen, sulfur, and phosphorous. In certain embodiments, said modified photosynthetic organism secretes an increased amount of a carbon-containing compound under said stress condition as compared to the wild-type microorganism under said stress condition, or as compared to a corresponding modified photosynthetic microorganism under a non-stress condition.

In particular embodiments wherein said modified photosynthetic organism has reduced expression of one or more genes of a glycogen biosynthesis or storage pathway as compared to the wild-type photosynthetic organism, said one or more genes are selected from the group consisting of: a glucose-1-phosphate adenyltransferase (glgC) gene, a phosphoglucomutase (pgm) gene, and a glycogen synthase (glgA) gene. In certain embodiment, said one or more genes comprise a complete or partial gene deletion. In particular embodiments, said photosynthetic organism is a Cyanobacterium.

Examples of carbon-containing compounds include lipids, such as fatty acids, optionally free fatty acids, triglycerides, wax esters, fatty alcohols, and alkanes/alkenes, in addition to other specialty chemicals described herein, such as certain biofuels, 2-oxoglutarate, pyruvate, malate, fumarate, succinate, 4-hydroxybutyrate, 1,4 butanediol, glutaconic acid, 3-methyl-2-oxobutyrate, 3-methyl-2-oxovalerate, 4-methyl-2-oxopentanoate, isobutaraldehyde, isobutanol, 2-methyl-1-butanol, 3-methyl-2-butanol, isopentanol, glucose, glutathione, 3-phosphoglycerate, cis-aconitate, glycerin, and polyamine intermediates such as agmatine and putrescine. In certain embodiments, the carbon-containing compound is a precursor or intermediate of a specialty chemical, such as a feedstock for a biofuel, e.g., a lipid.

In certain embodiments, an introduced polynucleotide is exogenous to the photosynthetic microorganism's native genome, e.g., it may be a polynucleotide derived from a different species. In other embodiments, the introduced polynucleotide is a polynucleotide native to the photosynthetic microorganism's genome, i.e., corresponding to a gene or protein normally present in the photosynthetic microorganism, but it is overexpressed, for example, from a recombinantly introduced expression vector. In certain embodiments, the vector is an inducible vector. In particular embodiments, an introduced polynucleotide is present in the photosynthetic microorganism either transiently or stably. Thus, in various embodiments, the introduced polynucleotide is introduced into the photosynthetic microorganism or an ancestor thereof.

In further related embodiments, the present invention includes a modified photosynthetic microorganism having a reduced level of expression of one or more genes of a glycogen biosynthesis or storage pathway as compared to the level of expression of the one or more genes in a wild-type photosynthetic microorganism, and which also comprises one or more introduced polynucleotides encoding proteins of a glycogen breakdown pathway or a functional fragment or variant thereof.

In particular embodiments, modified photosynthetic microorganisms of the present invention, e.g., Cynanobacteria, synthesize or accumulate a reduced amount of glycogen under stress conditions as compared to a wild-type photosynthetic microorganism. In related embodiments, these photosynthetic microorganisms secrete or intracellularly accumulate an increased amount of one or more carbon-containing compounds as compared to a wild-type photosynthetic microorganism grown under a comparable stress condition. In certain embodiments, the stress conditions are reduced nitrogen conditions. In various other embodiments, modified photosynthetic microorganisms of the present invention synthesize or accumulate a reduced amount of glycogen and/or an increased amount of a (non-glycogen) carbon-containing compound as compared to a corresponding wild-type photosynthetic microorganism grown under said stress conditions, or as compared to the same or comparable modified photosynthetic microorganism grown under non-stress conditions.

In certain embodiments, the one or more genes having reduced expression in a modified photosynthetic microorganism of the present invention are selected from glucose-1-phosphate adenyltransferase (glgC), phosphoglucomutase (pgm), and/or glycogen synthase (glgA). In particular embodiments, the modified photosynthetic microorganism comprises a mutation of one or more genes of a glycogen biosynthesis or storage pathway. In one specific embodiment, the photosynthetic microorganism comprises mutations of the glgC gene or the pgm gene. In one specific embodiment, the photosynthetic microorganism comprises mutations of the glgC gene and the pgm gene. In various embodiments, the mutations are complete or partial gene deletions.

In particular embodiments, the modified photosynthetic microorganism is a Synechococcus elongatus. In one embodiment, the Synechococcus elongatus is strain PCC 7942. In certain embodiments, the modified photosynthetic microorganism is a salt tolerant variant of S. elongatus PCC 7942. In other embodiments, the modified photosynthetic microorganism is Synechococcus sp. PCC 7002 or Synechocystis sp. PCC 6803.

In another related embodiment, the present invention provides a method of producing a carbon-containing compound other than glycogen, comprising producing said carbon-containing compound in a modified photosynthetic microorganism, e.g., a Cyanobacterium, having a reduced level of expression of one or more genes of a glycogen biosynthesis or storage pathway and/or comprising one or more polynucleotides encoding a protein of a glycogen breakdown or glycogen precursor secretion pathway or a functional fragment or variant thereof, wherein said modified photosynthetic microorganism is grown or cultured under a stress condition, e.g., reduced nitrogen conditions. In certain embodiments, the photosynthetic microorganism maintains photosynthesis but has reduced growth, relative to the same microorganism grown under non-stress conditions. In certain embodiments, the photosynthetic microorganism accumulates or secretes an increased amount of said carbon-containing compound as compared to a wild-type photosynthetic microorganism grown under said stress condition. In particular embodiments, the one or more genes are glucose-1-phosphate adenyltransferase (glgC), phosphoglucomutase (pgm), and/or glycogen synthase (glgA). In one particular embodiment, the photosynthetic microorganism comprises mutations in the one or more genes having reduced expression. In particular embodiments, the genes include the glgC gene and/or the pgm gene. In some embodiments, the mutations are complete or partial gene deletions.

In particular embodiments of the methods of the present invention, the photosynthetic microorganism is a Cyanobacterium. In certain embodiments, the Cyanobacterium is a Synechococcus elongatus. In one embodiment, the Synechococcus elongatus is strain PCC 7942. In certain embodiments, the modified photosynthetic microorganism is a salt tolerant variant of S. elongatus PCC 7942. In other embodiments, the modified photosynthetic microorganism is Synechococcus sp. PCC 7002 or Synechocystis sp. PCC 6803.

In certain embodiments, any of the modified photosynthetic microorganisms described above further comprise one or more additional modifications. As one example, such modified microorganisms may further comprise one or more introduced or overexpressed polynucleotides encoding one or more proteins associated with lipid biosynthesis. In certain aspects, the one or more proteins associated with lipid biosynthesis include an acyl-ACP reductase, acyl carrier protein (ACP), acyl ACP synthase (Aas), alcohol dehydrogenase, aldehyde dehydrogenase, aldehyde decarbonylase, thioesterase (TES), acetyl coenzyme A carboxylase (ACCase), diacylglycerol acyltransferase (DGAT), phosphatidic acid phosphatase (PAP; or phosphatidate phosphatase), triacylglycerol (TAG) hydrolase, fatty acyl-CoA synthetase, lipase/phospholipase, or any combination thereof.

In certain embodiments, the one or more enzymes associated with lipid biosynthesis comprises a diacylglycerol acyltransferase (DGAT), and the carbon-containing compounds produced by the modified photosynthetic microorganism comprise a triglyceride. In these and related embodiments, for instance, to increase production of triglycerides, the modified photosynthetic microorganism may further comprise one or more introduced or overexpressed polynucleotides encoding an acyl-ACP reductase, aldehyde dehydrogenase, phosphatidate phosphatase (PAP), acetyl coenzyme A carboxylase (ACCase), acyl carrier protein (ACP), phospholipase B, phospholipase C, fatty acyl Co-A synthetase, or any combination thereof. In specific embodiments, the modified photosynthetic microorganism comprises a DGAT in combination with an ACCase. In particular embodiments, the modified photosynthetic microorganism comprises a DGAT in combination with a PAP. In further embodiments, the modified photosynthetic microorganism comprises a DGAT in combination with an ACCase and a PAP.

In particular embodiments, the one or more enzymes associated with lipid biosynthesis comprises an acyl-ACP reductase. In some aspects, the expression or overexpression of an acyl-ACP reductase increases production of fatty acids, such as free fatty acids. In certain instances, such modified photosynthetic microorganisms may further comprise one or more introduced or overexpressed polynucleotides encoding an aldehyde dehydrogenase, to further increase production of fatty acids. In these and related embodiments, the modified photosynthetic microorganism may further comprise reduced expression of an aldehyde decarbonylase, reduced expression of an endogenous alcohol dehydrogenase, or both, to respectively shunt carbon away from alkanes and fatty alcohols and towards fatty acids.

In some embodiments, the one or more enzymes associated with lipid biosynthesis comprises an acyl-ACP reductase in combination with a diacylglyceroltransferase (DGAT), and the carbon-containing compounds produced by the modified photosynthetic microorganism comprise a triglyceride. In these and related embodiments, the modified photosynthetic microorganism may further comprise reduced expression of an endogenous aldehyde decarbonylase, to shunt carbon away from alkanes and towards fatty acids and thus triglycerides. In some embodiments, the DGAT may have wax ester synthase activity and the modified photosynthetic microorganism may further comprise an introduced or overexpressed alcohol dehydrogenase, where the carbon-containing compounds produced by the microorganism comprise a wax ester. In these and related embodiments, the modified photosynthetic microorganism may further comprise reduced expression of an endogenous aldehyde dehydrogenase, reduced expression of an aldehyde decarbonylase, or both, to respectively shunt carbon away from fatty acids and alkanes and towards wax esters.

In some embodiments, the one or more enzymes associated with lipid biosynthesis comprises an acyl-ACP reductase in combination with an alcohol dehydrogenase, wherein the carbon-containing compounds produced by the microorganism comprise a fatty alcohol. In these and related embodiments, the modified photosynthetic microorganism may further comprise reduced expression of an endogenous aldehyde decarbonylase, an endogenous aldehyde dehydrogenase, or both, to respectively shunt carbon away from alkanes and fatty acids and towards fatty alcohols.

In some embodiments, the one or more enzymes associated with lipid biosynthesis comprises an acyl-ACP reductase in combination with an aldehyde decarbonylase, wherein the carbon-containing compounds produced by the microorganism comprise an alkane. In these and related embodiments, the modified photosynthetic microorganism may further comprise reduced expression of an endogenous aldehyde dehydrogenase, reduced expression of an endogenous alcohol dehydrogenase, or both, to respectively shunt carbon away from fatty acids and fatty alcohols and towards alkanes.

In certain embodiments, the modified photosynthetic microorganisms described herein further comprise one or more of the following: (i) one or more overexpressed (e.g., introduced) polynucleotides encoding (a) an acyl carrier protein (ACP), (b) an acetyl coenzyme A carboxylase (ACCase), (c) a diacylglycerol acyltransferase (DGAT) optionally in combination with a fatty acyl Co-A synthetase, (d) an aldehyde dehydrogenase, (e) an alcohol dehydrogenase that is capable of converting a fatty aldehyde into a fatty alcohol optionally in combination with a wax ester synthase (e.g., DGAT having wax ester synthase activity), (f) a thioesterase, (g) an acyl-ACP reductase; or (h) any combination of (a)-(g); (ii) reduced expression of one or more genes encoding an endogenous aldehyde decarbonylase; (iii) reduced expression of one or more genes encoding an acyl-ACP synthetase (Aas), or (iv) any combination of (i)-(iii).

Certain embodiments relate to methods for providing secretion of glucose from a photosynthetic microorganism, comprising culturing a modified photosynthetic organism in a media under a stress condition, wherein said photosynthetic microorganism: (a) accumulates a reduced amount of glycogen as compared to the wild-type photosynthetic microorganism, and (b) comprises one or more introduced or (over)expressed polynucleotides encoding a glucose permease, wherein said modified photosynthetic organism maintains photosynthetic activity and accumulates reduced biomass when grown under said stress condition as compared to when grown under non-stress conditions.

Some embodiments relate to methods for producing isobutanol or isopentanol, comprising culturing a modified photosynthetic organism in a media under a stress condition, wherein said photosynthetic microorganism: (a) accumulates a reduced amount of glycogen as compared to the wild-type photosynthetic microorganism, and (b) comprises one or more introduced or overexpressed polynucleotides encoding one or more polypeptides associated with production of isobutanol or isopentanol, wherein said modified photosynthetic microorganism maintains photosynthetic activity and accumulates reduced biomass when grown under said stress condition as compared to when grown under non-stress conditions. In particular embodiments, said one or more polypeptides of (b) are selected from a gene that converts a 2-keto acid to an aldehyde (2-keto acid decarboxylase) and a gene that converts the aldehyde to an alcohol (alcohol dehydrogenase).

Certain embodiments relate to methods for producing 4-hydroxybutyrate, comprising culturing a modified photosynthetic organism in a media under a stress condition, wherein said photosynthetic microorganism: (a) accumulates a reduced amount of glycogen as compared to the wild-type photosynthetic microorganism, and (b) comprises one or more introduced or overexpressed polynucleotides encoding one or more polypeptides associated with production of 4-hydroxybutyrate, wherein said modified photosynthetic microorganism maintains photosynthetic activity and accumulates reduced biomass when grown under said stress condition as compared to when grown under non-stress conditions. In specific embodiments, said one or more polypeptides of (b) are an alpha ketoglutarate decarboxylase, a 4-hydroxybutyrate dehydrogenase, a succinyl-CoA synthetase, a succinate-semialdehyde dehydrogenase, or any combination thereof. Also included are methods for producing 1,4-butanediol, wherein said photosynthetic microorganism: (c) further comprises one or more introduced or overexpressed polynucleotides encoding one or more polypeptides associated with production of 1,4-butanediol from 4-hydroxybutyrate. In some embodiments, said one or more polypeptides of (c) are a 4-hydroxybutyryl-CoA transferase, an aldehyde/alcohol dehydrogenase that is optionally capable of reducing coA-linked substrates to aldehydes/alcohols, or both.

Particular embodiments relate to methods method of producing a polyamine intermediate, comprising culturing a modified photosynthetic organism in a media under a stress condition, wherein said photosynthetic microorganism: (a) accumulates a reduced amount of glycogen as compared to the wild-type photosynthetic microorganism, and (b) optionally comprises one or more introduced or overexpressed polynucleotides encoding one or more polypeptides associated with production of a polyamine intermediate, wherein said modified photosynthetic organism maintains photosynthetic activity and accumulates reduced biomass when grown under said stress condition as compared to when grown under non-stress conditions. In some embodiments, said polyamine intermediate is putrescine or agmatine. In some embodiments, said one or more polypeptides is an arginine decarboxylase, and said polyamine intermediate is agmatine. In specific embodiments, said one or more polypeptides is an arginine decarboxylase, an agmatine deiminase, an N-carbamoylputrescine amidase, or any combination thereof, and said polyamine intermediate is putrescine.

In certain embodiments, an overexpressed polypeptide is encoded by (i) an endogenous polynucleotide which is operably linked to one or more introduced regulatory elements, or (ii) an introduced polynucleotide. In particular embodiments, the one or more introduced regulatory elements or introduced polynucleotides are exogenous to the photosynthetic microorganism's native genome. In certain embodiments, one or more introduced regulatory elements or introduced polynucleotides are derived from the same genus as said modified photosynthetic microorganism. In specific embodiments, said one or more introduced regulatory elements or introduced polynucleotides are derived from the same species as said modified photosynthetic microorganism. In some embodiments, said one or more introduced regulatory elements or introduced polynucleotides are derived from a different genus or species relative to said modified photosynthetic microorganism. In particular embodiments, said one or more introduced regulatory elements are selected from at least one of a promoter, enhancer, repressor, ribosome binding site, and a transcription termination site.

In certain embodiments, said one or more introduced regulatory elements comprises an inducible promoter. In some embodiments, said inducible promoter is a weak promoter under non-induced conditions. In certain embodiments, said one or more introduced regulatory elements comprises a constitutive promoter.

In certain embodiments, one or more of said introduced polynucleotides is present in one or more expression constructs. In certain embodiments, said expression construct is stably integrated into the genome of said modified photosynthetic microorganism. In certain embodiments, said expression construct comprises an inducible promoter. In some embodiments, one or more of said introduced polynucleotides are present in an expression construct comprising a weak promoter under non-induced conditions.

In certain embodiments, one or more of said introduced polynucleotides are codon-optimized for expression in a Cyanobacterium. In particular embodiments, one or more of said codon-optimized polynucleotides are codon-optimized for expression in a Synechococcus elongatus. In certain embodiments, said photosynthetic microorganism is a Cyanobacterium and said Cyanobacterium is a Synechococcus elongatus. In specific embodiments, said Synechococcus elongatus is strain PCC7942. In certain embodiments, said Cyanobacterium is a salt tolerant variant of Synechococcus elongatus strain PCC7942. In other embodiments, said photosynthetic microorganism is a Cyanobacterium and said Cyanobacterium is Synechococcus sp. PCC7002. In certain embodiments, said photosynthetic microorganism is a Cyanobacterium and said Cyanobacterium is Synechocystis sp. PCC6803.

As noted above, also included are methods for the production of carbon-containing compounds such as lipids, comprising culturing a modified photosynthetic microorganism described herein under stress conditions, wherein said modified photosynthetic microorganism accumulates an increased amount of carbon-containing compound as compared to a corresponding wild-type photosynthetic microorganism grown under said stress condition, and wherein said modified photosynthetic microorganism maintains photosynthesis but has reduced growth. In certain embodiments, said culturing comprises inducing expression of one or more of said introduced polynucleotides. In some embodiments, said culturing comprises culturing under static growth conditions. In certain embodiments, said inducing occurs under static growth conditions.

Certain of the methods and systems described herein include the step of relieving the stress condition, for instance, when the ratio of absorbance (680/750 nm) of the culture is (or falls to) about 10%-90% of the ratio of a corresponding culture under non-stress conditions, where relieving the stress condition increases photosynthetic activity of the modified photosynthetic microorganism and/or increases the ratio of absorbance of the culture. In some aspects, the stress condition comprises reduced or depleted levels of an essential nutrient (e.g., nitrogen, phosphorous, sulfur), and the methods include adding (i.e., pulsing the culture with) the essential nutrient in an amount sufficient to increase photosynthetic activity and/or increase the ratio of absorbance. In certain embodiments, said photosynthetic activity increases by at least about 10% relative to photosynthetic activity immediately prior to relief of said stress condition. In particular embodiments, the modified photosynthetic microorganism maintains the increased photosynthetic activity for a substantially longer time (e.g., about 1.5, 2, 3, 4, 5, 6, 7, 8, 9, 10 or more times longer) than a wild-type photosynthetic microorganism under the same or comparable culture conditions. In certain aspects, the ratio of absorbance increases to greater than about 90% of the ratio of a corresponding culture under non-stress conditions, where non-stress conditions optionally comprise nutrient replete conditions. In most instances, the modified photosynthetic microorganism culture maintains the increased ratio of absorbance for a substantially longer time (e.g., about 1.5, 2, 3, 4, 5, 6, 7, 8, 9, 10 or more times longer) than a wild-type photosynthetic microorganism culture under the same or comparable culture conditions. After relief of the stress condition and an initial increase in photosynthetic activity, there is often a subsequent decrease in photosynthetic activity, and in certain aspects the decrease in photosynthetic activity by the modified photosynthetic microorganism is substantially less (e.g., about 1.5, 2, 3, 4, 5, 6, 7, 8, 9, 10 or more times less) than the subsequent decrease in photosynthetic activity by a wild-type photosynthetic microorganism culture under the same or comparable culture conditions. Certain embodiments further comprise repeating the step of relieving the stress condition when the ratio of absorbance falls (again) to about 10%-90% of the ratio of a corresponding culture under non-stress conditions.

Certain of the methods and systems described herein include the step of relieving the stress condition, for instance, at about 2, 3, 4, 5, 6, 7, 8, 9, 10, 11, 12, 13, or 14 days following initiation of the stress condition, where relieving the stress condition increases photosynthetic activity. In certain aspects, the stress condition comprises reduced level of an essential nutrient, and relieving the stress condition comprises adding (i.e., pulsing the culture with) the essential nutrient in an amount sufficient to increase photosynthetic activity. In some embodiments, said photosynthetic activity increases by at least about 10% relative to photosynthetic activity immediately prior to relief of said stress condition. In particular embodiments, the modified photosynthetic microorganism maintains the increased photosynthetic activity for a substantially longer time than a wild-type photosynthetic microorganism under the same or comparable culture conditions. Similar to above, there is often a subsequent decrease in photosynthetic activity following relief of the stress condition and an initial increase in photosynthetic activity, and in certain aspects the decrease in photosynthetic activity by the modified photosynthetic microorganism is substantially less (e.g., about 1.5, 2, 3, 4, 5, 6, 7, 8, 9, 10 or more times less) than the subsequent decrease in photosynthetic activity by a wild-type photosynthetic microorganism culture under the same or comparable culture conditions. Certain embodiments further comprise repeating the step of relieving the stress condition about every 2, 3, 4, 5, 6, 7, 8, 9, 10, 11, 12, 13, or 14 days following previous relief of the stress condition, where relieving the stress condition increases photosynthetic activity.

In certain embodiments, the essential nutrient is selected from at least one of nitrogen, sulfur, and phosphorous. In specific embodiments, the essential nutrient is nitrogen, which can added, for example, in the form of NaNO₃, NH₄Cl, (NH₄)₂SO₄, NH₄HCO₃, CH₄N₂O, KNO₃, or any combination thereof, optionally to achieve a final concentration ranging from about 0.02 mM to about 20 mM to about 30 mM.

In certain embodiments, the photosynthetic activity of the modified photosynthetic microorganism under the stress condition is least about 20% of photosynthetic activity of the modified photosynthetic microorganism or the wild-type photosynthetic microorganism under non-stress conditions. In particular embodiments, the photosynthetic activity of the modified photosynthetic microorganism under the stress condition is least about 50% of photosynthetic activity of the modified photosynthetic microorganism or the wild-type photosynthetic microorganism under non-stress conditions. In certain embodiments, the photosynthetic activity of the modified photosynthetic microorganism under the stress condition is substantially greater (e.g., at least about 1.5, 2, 3, 4, 5, 6, 7, 8, 9, 10 or 20-fold greater) than photosynthetic activity of the wild-type photosynthetic microorganism under the stress condition. In certain aspects, said photosynthetic activity is measured at about day 2, 3, 4, 5, 6, 7, 8, 9, 10, 11, 12, 13, or 14 post-initiation of the stress condition.

In some of the methods and systems described herein, the maintenance of photosynthetic activity comprises maintenance of chlorophyll A levels. In particular embodiments, chlorophyll A levels of the modified photosynthetic microorganism under the stress condition are at least about 20% of chlorophyll A levels of the modified photosynthetic microorganism or the wild-type photosynthetic microorganism under non-stress conditions. In some embodiments, chlorophyll A levels of the modified photosynthetic microorganism under the stress condition are at least about 50% of chlorophyll A levels of the modified photosynthetic microorganism or the wild-type photosynthetic microorganism under non-stress conditions. In certain embodiments, chlorophyll A levels of the modified photosynthetic microorganism under the stress condition are substantially greater (e.g., at least about 1.5, 2, 3, 4, 5, 6, 7, 8, 9, 10 or 20-fold greater) than chlorophyll A levels of the wild-type photosynthetic microorganism under the stress condition. In certain aspects, chlorophyll A levels are measured at about day 2, 3, 4, 5, 6, 7, 8, 9, 10, 11, 12, 13, or 14 post-initiation of the stress condition.

### BRIEF DESCRIPTION OF THE SEVERAL VIEWS OF THE DRAWINGS

FIG. 1 shows measurements of S. elongatus photosynthetic complexes during nitrogen starvation, comparing wild-type to glycogen synthesis mutants.

FIGS. 2a and 2b show attenuation of the NtcA-mediated transcriptional response in nitrogen starved glgC mutants.

FIG. 3 shows nitrogen deprivation of glgC mutant for 24 hours triggers excretion of 2-oxoglutarate equivalent to 10-15% of culture dry weight. Cultures of wild type (WT) and ΔglgC were resuspended in either nitrogen replete (1×N) or media depleted entirely of nitrate (0×N) and maintained in constant light at 30° C. for 24 hours. 2-oxoglutarate level (μM) in the culture media was monitored for each sample by enzymatic assay at the specified times. Data was generated in triplicate and error is expressed as standard deviation of the mean. Samples whose mean fell below the 1 μM detection limit of the assay are not graphed.

FIG. 4 shows elimination of glycogen synthesis alters expression of nblA in response to nitrogen stress.

FIGS. 5a-5c show that KgtP-facilitated internalization of 2-oxoglutarate delays nblA-luxAB reporter activation and phycobilisome degradation in response to nitrogen stress.

FIG. 6 shows chlorophyll A levels for wild-type S. elongatus and glgC mutant following nitrogen starvation, indicated as a percentage of wild-type chlA levels under non-stress (nitrogen replete) conditions.

FIGS. 7a and 7b show a non-chlorotic phenotype of ΔglgC in response to nitrogen stress.

FIG. 8 shows that ΔglgA exhibits a non-chlorotic phenotype in response to nitrogen stress.

FIG. 9 shows that nitrogen stress triggers immediate growth arrest in the ΔglgC mutant.

FIG. 10 shows that deletion of glgC eliminates glycogen synthesis in S. elongatus.

FIG. 11 shows that deletion of glgC does not alter the reduction in O₂ evolution activity observed in S. elongatus in response to nitrogen stress.

FIGS. 12a and 12b show transformation of ΔglgC with glgC transgene (glgC^{TG}) restores chlorosis and glycogen synthesis under nitrogen stress.

FIGS. 13a-13d show a non-chlorotic phenotype of ΔglgC in response to sulfur and phosphate stress.

FIG. 14 shows the amount of 2-oxoglutarate secreted each day by the glgC and glgA mutants post nitrogen starvation.

FIGS. 15a-15e show secretion of carbon skeletons into the culture media by nitrogen starved glgC mutants.

FIGS. 16a-16c show that accumulation of metabolites in a nitrogen starved glgC mutant can be converted to metabolic precursors of volatile biofuel end products.

FIGS. 17a-17i show increased production of carbon-containing compounds by a nitrogen starved glgC mutant, including amino acid precursor metabolites, TCA cycle metabolites, and glycolysis metabolites.

FIGS. 18a-18b show increased production of carbon-containing compounds by a nitrogen starved glgC mutant, including polyamine metabolites agmatine and putrescine. Cultures of wild type and ΔglgC were resuspended in either nitrogen replete (1×N) or media depleted entirely of nitrate (0×N).

FIGS. 19a-19c show elimination of glycogen synthesis triggers excretion of TCA cycle intermediates in response to nitrogen stress.

FIG. 20 shows elimination of glycogen synthesis alters intracellular partitioning of glycolytic and TCA cycle intermediates in response to nitrogen starvation.

FIG. 21 illustrates certain of the metabolites that are increased (bold) or decreased (bold italics) in nitrogen starved mutants that accumulate a reduced amount of glycogen, and some the carbon-containing compounds that can be produced therefrom.

FIG. 22 shows biosynthetic pathways for the production of 4-hydroxybutyrate and 1,4-butanediol.

FIG. 23 shows a biosynthetic pathway for the production of putrescine, a polyamine.

### DETAILED DESCRIPTION

The present invention is based, in part, upon the discovery that certain modified photosynthetic microorganisms, e.g., Cyanobacteria, modified to have reduced glycogen accumulation (e.g., due to decreased production or increased degradation or secretion), are capable of producing carbon-containing compounds and maintaining photosynthesis under stress conditions that otherwise inhibit or reduce their growth. Accordingly, under such stress conditions, glycogen reduced or deficient photosynthetic microorganisms may be used for continuous photosynthetic production of carbon-containing compounds without biomass growth.

Cyanobacteria are photosynthetic bacteria that obtain energy and reducing power from sunlight and water to reduce CO₂ to carbohydrates. Synechococcus elongatus PCC 7942 is a genetically malleable cyanobacterium that is reasonably well studied with regard to its physiology and molecular biology. However, during algal production of carbon-containing compounds, large amounts of carbon and energy are used for accumulation of cellular biomass, and this is a significant drain on cellular resources that could otherwise be used for production of any given carbon-containing compound. Limiting or eliminating general biomass accumulation while maintaining photosynthetic activity and production of desired end products has the potential to increase the efficiency and yield of cyanobacteria-based production platforms.

Another challenge to algal productions systems is that desirable end products are accumulated inside cells. This requires periodic harvesting of whole biomass from the production system to extract the product of interest followed by regrowth of another batch of biomass. The carbon, energy, and nutrients required to generate this biomass are lost if the biomass is not recycled into the next round of growth. These recycling processes are not well developed and require a significant energy input which negatively impacts the net energy that can be obtained from algal biofuel production systems and greatly increases the cost of production.

The accompanying description and Examples describe S. elongatus strains and growth conditions that allow for a continuous production platform that consists of both internal and secreted end products combined with minimal biomass accumulation during the production phase. This end was achieved by the combination of growth conditions that limit nutrients necessary for biomass accumulation, such as nitrogen depletion, along with gene mutations that prohibit glycogen storage and thereby alter the physiological response and global carbon flow under nutrient limitation. For example, without wishing to be bound to any particular theory, it is understood that in wild-type cells, upon nitrogen limitation, levels of the TCA cycle metabolite 2-oxoglutarate increase inside the cell, due to an inability to convert this metabolite into the amino acids glutamate and glutamine. As 2-oxoglutarate levels increase it activates a genetic program through multiple regulatory proteins, such as NtcA and PII (glnK). NtcA and PII bind to 2-oxoglutarate and regulate transcription of multiple genes, resulting in the degradation of photosynthetic complexes and the storage of carbon as glycogen. For wild-type cells, this process results in a decreased ability to photosynthesize and convert carbon dioxide to carbon-containing compounds such as biofuel end products, or other specialty chemicals or intermediates thereof.

The invention described herein allows maintenance of photosynthetic activity of photosynthetic microorganisms such as Synechococcus elongatus PCC 7942 during nutrient starvation by generation of a strain that suppresses the normal physiological response to nutrient limitation, does not store carbon as glycogen, and results in the intracellular accumulation of carbon-containing compounds and/or secretion of carbon-containing compounds from the cells into the media. The fact that photosynthesis and cell growth could be uncoupled under stress conditions by reducing glycogen biosynthesis is both surprising and unexpected. In addition, this surprising discovery affords major advantages, since it allows for the continuous photosynthetic production of carbon-containing compounds, while reducing cell growth and associated shading effects that result at high cell density. In addition, this discovery reduces the frequency with which a culture must be divided or its density reduced.

Among other combinations described herein, embodiments of the present invention may be combined with the discovery that expression or overexpression of certain genes involved in lipid biosynthesis leads to higher levels of lipid biosynthesis. Thus, in certain embodiments, modified photosynthetic microorganisms, e.g., Cyanobacteria, used according to the present invention further comprise one or more exogenous (i.e., introduced) or overexpressed polynucleotides that encode a lipid biosynthesis protein. Specific examples of lipid biosynthesis proteins include acyl-ACP reductases, acyl carrier proteins (ACP), acyl-ACP synthases (Aas), thioesterases or acyl-ACP thioesterases (TES) such as TesA or FatB, diacylglycerol acyltransferases (DGAT), acetyl coenzyme A carboxylases (ACCase), phosphatidic acid phosphatases (PAP; or phosphatidate phosphatases), triacylglycerol (TAG) hydrolases or lipases, fatty acyl-CoA synthetases, aldehyde dehydrogenases, alcohol dehydrogenases, aldehyde decarbonylases, lipases, and phospholipases (PL) such as phospholipase A, B, or C. In certain instances, as described herein, reduced expression and/or activity of selected lipid biosynthesis genes can increase production of one or more desired lipids, for instance, by shunting carbon towards the desired lipid(s). Hence, various combinations of overexpressed and/or reduced lipid biosynthesis proteins can be employed to optimize production of selected lipids relative to others, such as fatty acids, triglycerides, wax esters, fatty alcohols, and alkanes/alkenes. Lipid biosynthesis proteins are described in greater detail below.

Aspects of the present invention can also be combined with the discovery that photosynthetic microorganisms such as Cyanobacteria can be genetically modified in other ways to increase the production of fatty acids, as described herein and in International Patent Applications US2009/061936 and PCT/US2011/065896 and U.S. patent application Ser. No. 12/605,204.

For instance, as described in PCT/US2011/065896, (over)expression of an acyl-ACP reductase (e.g., orf1594) has been shown to increase production of fatty aldehydes, which can then be converted to fatty acids by an aldehyde dehydrogenase. Hence, in certain aspects, an introduced or overexpressed acyl-ACP reductase, optionally in combination with an introduced or overexpressed aldehyde dehydrogenase, can be employed to further increase the production of fatty acids under stress conditions. The introduction or overexpression of an acyl-ACP reductase can be combined with expression and/or reduction (e.g., deletion) of various combinations of lipid biosynthesis proteins, described herein, to selectively increase production of certain lipids relative to others, such as fatty acids, triglycerides, wax esters, fatty alcohols, and alkanes/alkenes. Merely by way of illustration, an introduced or overexpressed acyl-ACP reductase can be combined with an introduced or overexpressed aldehyde dehydrogenase to increase fatty acids and optionally triglycerides (e.g., when further combined with an introduced or overexpressed DGAT), an introduced or overexpressed aldehyde decarbonylase to increase alkanes/alkenes, or an introduced or overexpressed alcohol dehydrogenase to increase production of fatty alcohols and optionally triglycerides/wax esters (e.g., when further combined with an introduced or overexpressed DGAT), among other combinations described herein.

In a variety of aspects, embodiments of the present invention are also useful in combination with the related discovery that photosynthetic microorganisms, including Cyanobacteria, such as Synechococcus, which do not naturally produce triglycerides, can be genetically modified to synthesize triglycerides, as described herein and in International Patent Application US2009/061936 and U.S. patent application Ser. No. 12/605,204, filed Oct. 23, 2009, titled Modified Photosynthetic Microorganisms for Producing Triglycerides. For instance, the addition of one or more polynucleotide sequences that encode one or more enzymes associated with triglyceride synthesis renders Cyanobacteria capable of converting their naturally-occurring fatty acids into triglyceride energy storage molecules. Examples of enzymes associated with triglyceride synthesis include enzymes having a phosphatidate phosphatase activity (PAP) and enzymes having a diacylglycerol acyltransferase activity (DGAT). Specifically, phosphatidate phosphatase enzymes catalyze the production of diacylglycerol molecules, an immediate pre-cursor to triglycerides, and DGAT enzymes catalyze the final step of triglyceride synthesis by converting the diacylglycerol precursors to triglycerides. Hence, recombinant introduction or overexpression of DGAT, PAP, or both, can be utilized to produce triglycerides under stress conditions. Further, because fatty acids provide the starting material for triglycerides, increasing the production of fatty acids in genetically modified photosynthetic microorganisms may be utilized to increase the production of triglycerides, for instance, by increasing ACCase and/or acyl-ACP reductase activity, as described herein and in International Patent Applications PCT/US2009/061936 and PCT/US2011/065896.

In particular aspects, embodiments of the present invention may also be combined with the discovery that the co-expression of an acyl-ACP reductase, alcohol dehydrogenase and a DGAT or other polypeptide having wax ester synthase activity results in wax ester formation, via the acyl-ACP=>fatty aldehyde pathway. For instance, Cyanobacteria over-expressing an acyl-ACP reductase (e.g., orf1594), a long chain alcohol dehydrogenase, and the bi-functional aDGAT enzyme not only produce fewer triglycerides, but also produce wax esters. Because these modified Cyanobacteria produce free fatty acids, and thus suggest that endogenous aldehyde dehydrogenase encoded by orf0489 competes with alcohol dehydrogenase for acyl aldehyde substrate, reduced expression (e.g., deletion) of orf0489 may increase wax ester synthesis in these and related microorganisms, relative to modified microorganisms having no reduced expression of orf0489. Further, because aldehyde decarbonylase encoded by orf1593 may also compete with alcohol dehydrogenase for acyl aldehyde substrate, reduced expression (e.g., deletion) of orf1593 may independently increase wax ester synthesis, and when combined with reduced expression of orf0489 may even further increase wax ester synthesis. Increased wax ester formation may also be achieved by combining any one of these or related embodiments with overexpression of other genes related to fatty aldehyde synthesis, including acyl carrier protein (ACP), Aas, or both.

Other combinations are described herein and will be apparent to persons skilled in the art, including those that relate to the production of non-lipid carbon-containing compounds. Examples of such compounds include but are not limited to various amino acid precursor metabolites, TCA cycle metabolites such as 2-Oxoglutarate, succinatem, and fumarate, glycolysis metabolites such as glucose, isobutanol, isopentanol, 4-hydroxybutyrate, 1,4-butanediol, and polyamine intermediates such as agmatine and putrescine.

### DEFINITIONS

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The term “biologically active fragment”, as applied to fragments of a reference polynucleotide or polypeptide sequence, refers to a fragment that has at least about 0.1, 0.5, 1, 2, 5, 10, 12, 14, 16, 18, 20, 22, 24, 26, 28, 30, 35, 40, 45, 50, 55, 60, 65, 70, 75, 80, 85, 90, 95, 96, 97, 98, 99, 100, 110, 120, 150, 200, 300, 400, 500, 600, 700, 800, 900, 1000% or more of the activity of a reference sequence. The term “reference sequence” refers generally to a nucleic acid coding sequence, or amino acid sequence, to which another sequence is being compared. All sequences provided in the Sequence Listing are also included as reference sequences.

The term “biologically active variant”, as applied to variants of a reference polynucleotide or polypeptide sequence, refers to a variant that has at least about 0.1, 0.5, 1, 2, 5, 10, 12, 14, 16, 18, 20, 22, 24, 26, 28, 30, 35, 40, 45, 50, 55, 60, 65, 70, 75, 80, 85, 90, 95, 96, 97, 98, 99, 100, 110, 120, 150, 200, 300, 400, 500, 600, 700, 800, 900, 1000% or more of the activity (e.g., an enzymatic activity) of a reference sequence. The term “reference sequence” refers generally to a nucleic acid coding sequence, or amino acid sequence, to which another sequence is being compared. The term “variant” encompasses biologically active variants, which may also be referred to as functional variants.

Included within the scope of the present invention are biologically active fragments of at least about 18, 19, 20, 21, 22, 23, 24, 25, 26, 27, 28, 29, 30, 40, 50, 60, 70, 80, 90, 100, 120, 140, 160, 180, 200, 220, 240, 260, 280, 300, 320, 340, 360, 380, 400, 500, 600 or more contiguous nucleotides or amino acid residues in length, including all integers in between, which comprise or encode a polypeptide having an activity of a reference polynucleotide or polypeptide. Representative biologically active fragments and variants generally participate in an interaction, e.g., an intra-molecular or an inter-molecular interaction. An inter-molecular interaction can be a specific binding interaction or an enzymatic interaction. Examples of enzymatic interactions or activities include, without limitation, acyl-acyl carrier protein reductase activity, acyl carrier protein activity, glycogen breakdown activity, glycogen precursor secretion activity, glucose secretion activity, acetyl-CoA carboxylase activity, aldehyde dehydrogenase activity, alcohol dehydrogenase activity, aldehyde decarbonylase activity, and other enzymatic activities described herein.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties (e.g., pegylation) or by post-translational modification techniques as would be understood in the art. The term “derivative” also includes within its scope alterations that have been made to a parent sequence including additions or deletions that provide for functionally equivalent molecules.

By “enzyme reactive conditions” it is meant that any necessary conditions are available in an environment (i.e., such factors as temperature, pH, lack of inhibiting substances) which will permit the enzyme to function. Enzyme reactive conditions can be either in vitro, such as in a test tube, or in vivo, such as within a cell.

As used herein, an “acyl-acyl carrier protein reductase” (or “acyl-ACP reductase”) includes an enzyme that converts acyl-ACP to acyl-aldehyde.

As used herein, the terms “function” and “functional” and the like refer to a biological, enzymatic, or therapeutic function.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences).

“Homology” refers to the percentage number of amino acids that are identical or constitute conservative substitutions. Homology may be determined using sequence comparison programs such as GAP (Deveraux et al., 1984, Nucleic Acids Research 12, 387-395) which is incorporated herein by reference. In this way sequences of a similar or substantially different length to those cited herein could be compared by insertion of gaps into the alignment, such gaps being determined, for example, by the comparison algorithm used by GAP.

The term “host cell” includes an individual cell or cell culture which can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell which comprises a recombinant vector of the invention is a recombinant host cell.

By “isolated” is meant material that is substantially or essentially free from components that normally accompany it in its native state. For example, an “isolated polynucleotide”, as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

The terms “modulating” and “altering” include “increasing” and “enhancing” as well as “decreasing” or “reducing,” typically in a statistically significant or a physiologically significant amount or degree relative to a control. By “increased” or “increasing” is included the ability of one or more modified photosynthetic microorganisms, e.g., Cyanobacteria, to produce (e.g., intracellularly accumulate and/or secrete) a greater amount of one or more carbon-containing compounds when grown under a stress condition, relative to a control photosynthetic microorganism, typically of the same species, such as an unmodified (wild-type) photosynthetic microorganism or a differently modified photosynthetic microorganism grown under that same or a similar stress condition, or relative to the same or a similarly modified photosynthetic microorganism grown under a non-stress condition. In particular embodiments, the stress condition is reduced levels or absence of at least one essential nutrient, and the comparative non-stress condition is a replete condition of the same or all essential nutrient(s). Examples of carbon-containing compounds are described herein.

For lipids, included are increases in total lipids, total fatty acids, total free fatty acids, total intracellular fatty acids, and/or total secreted fatty acids, separately or together. For instance, in certain embodiments, total lipids may increase, with either corresponding increases in all types of lipids, or relative increases in one or more specific types of lipid (e.g., fatty acids, free fatty acids, secreted fatty acids, triglycerides, wax esters). In certain embodiments, total lipids may increase or they may stay the same (i.e., total lipids are not significantly increased compared to an unmodified microorganism of the same type), and the production or storage of fatty acids (e.g., free fatty acids, secreted fatty acids) may increase relative to other lipids. In particular embodiments, the production or storage of one or more selected types of fatty acids (e.g., secreted fatty acids, free fatty acids, intracellular fatty acids, specific fatty acids such as C14:0, C14:1, C16:0, C16:1n9, and C18:0 fatty acids) may increase relative to other types of fatty acids (e.g., secreted fatty acids, free fatty acids, intracellular fatty acids, specific fatty acids such as C14:0, C14:1, C16:0, C16:1n9, and C18:0 fatty acids).

An “increased” or “enhanced” amount is typically a “statistically significant” amount, and may include an increase that is 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein. Certain examples include the amount of carbon-containing compound produced by a corresponding unmodified microorganism or differently modified microorganism grown under the stress condition, or an amount produced by a corresponding modified photosynthetic microorganism grown under non-stress conditions. In specific embodiments, production or storage of a one or more carbon-containing compounds described herein, such as total lipids, total triglycerides, total fatty acids, total free fatty acids, selected fatty acids (e.g., C16:0) total intracellular fatty acids, total secreted fatty acids, and/or total wax esters, is increased relative to an unmodified or differently modified microorganism (e.g., for triglycerides, a DGAT-only expressing strain) grown under the stress condition, or relative to an amount produced by a corresponding modified photosynthetic microorganism grown under non-stress conditions, as described above, or by at least 10%, at least 15%, at least 20%, at least 25%, at least 30%, at least 40%, at least 50%, at least 60%, at least 70%, at least 80%, at least 90%, at least 100%, at least 150%, at least 200%, at least 300%, at least 400%, at least 500%, or at least 1000%. In certain embodiments, production or storage of total carbon-containing compounds such as total lipids, total triglycerides, total fatty acids, total free fatty acids, total intracellular fatty acids, total secreted fatty acids, and/or total wax esters is increased by about 50% to 200%.

Production of lipids such as fatty acids can be measured according to techniques known in the art, such as Nile Red staining, thin layer chromatography and gas chromatography. Production of triglycerides can be measured, for example, using commercially available enzymatic tests, including colorimetric enzymatic tests using glycerol-3-phosphate-oxidase. Production of free fatty acids can be measured in absolute units such as overall accumulation of FAMES (e.g., OD/ml, μg/ml) or in units that reflect the production of FAMES over time, i.e., the rate of FAMES production (e.g., OD/ml/day, μg/ml/day). For example, certain modified microorganisms described herein may produce at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 μg/mL/day; and/or in the range of at least about 20-30, 20-35, 20-40, 20-45, 20-50, 25-30, 25-35, 25-40, 25-45, 25-50, 30-35, 30-40, 30-45, 30-50, 35-40, 35-45, 35-50, 40-45, or 40-50 μg/mL/day. Production of TAGs can be measured similarly.

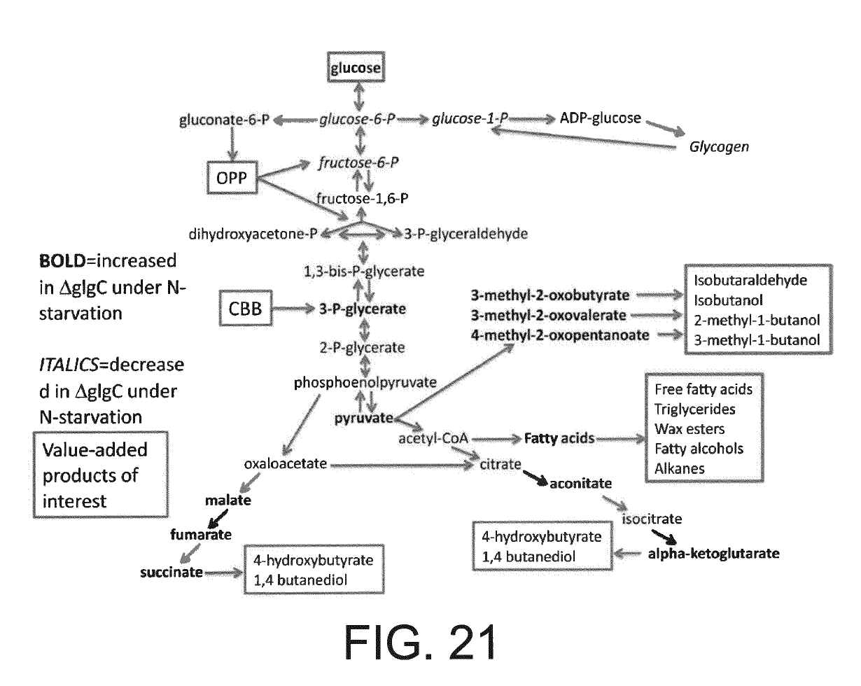

A “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein. In certain instances, by “decreased” or “reduced” is included the ability of one or more modified photosynthetic microorganisms, e.g., Cyanobacteria, to produce or accumulate under a stress condition a lesser amount (e.g., a statistically significant amount) of glycogen or glycogen precursor or related molecule (see FIG. 21), as compared to a control photosynthetic microorganism grown under the same or comparable stress condition, such as an unmodified or wild-type Cyanobacteria or a differently modified Cyanobacteria. Production of glycogen and related molecules can be measured according to techniques known in the art (see Suzuki et al., Biochimica et Biophysica Acta 1770:763-773, 2007). In certain instances, by “decreased” or “reduced” is meant a lesser level of expression (e.g., a statistically significant amount), by a modified photosynthetic microorganism, e.g., Cyanobacteria, of one or more genes associated with a glycogen biosynthesis or storage pathway, as compared to the level of expression in a control photosynthetic microorganism, such as an unmodified Cyanobacteria or a differently modified Cyanobacteria. In particular embodiments, production or accumulation of glycogen or glycogen precursor or related molecule, and/or expression of one or more genes associated with glycogen biosynthesis or storage, is reduced by at least 10%, at least 20%, at least 30%, at least 40%, at least 50%, at least 60%, at least 70%, at least 80%, at least 90%, or 100%. In particular embodiments, production or accumulation of glycogen or glycogen precursor or related molecule, and/or expression of one or more genes associated with glycogen biosynthesis or storage, is reduced by about 50-100%.

Examples of “carbon-containing compounds” include specialty chemicals and associated precursors such as lipids (e.g., fatty aldehydes, fatty acids, free fatty acids, triglycerides, wax esters, fatty alcohols, alkanes), 2-oxoglutarate, malate, fumarate, succinate, 3-methyl-2-oxobutyrate, 3-methyl-2-oxovalerate, 4-methyl-2-oxopentanoate, glucose, glutathione, 3-phosphoglycerate, cis-aconitate, pyruvate, 4-hydroxy-2-oxoglutaric acid, 3-P-glycerate, acetyl CoA, aconinate, 4-hydroxybutyrate, 1,4-butanediol, glutaconic acid, isobutyraldehyde, isobutanol, isopentanol, 2-methyl-1-butanol, 3-methyl-1-butanol, and polyamine intermediates such as agmatine and putrescine.

Additional examples of carbon-containing compounds or specialty chemicals and associated precursors include ethanol, biodiesel, methane, methanol, ethane, ethene, n-propane, 1-propene, 1-propanol, propanal, acetone, propionate, n-butane, 1-butene, 1-butanol, butanal, butanoate, isobutanal, isobutanol, 2-methylbutanal, 2-methylbutanol, 3-methylbutanal, 3-methylbutanol, 2-butene, 2-butanol, 2-butanone, 2,3-butanediol, 3-hydroxy-2-butanone, 2,3-butanedione, ethylbenzene, ethenylbenzene, 2-phenylethanol, phenylacetaldehyde, 1-phenylbutane, 4-phenyl-1-butene, 4-phenyl-2-butene, 1-phenyl-2-butene, 1-phenyl-2-butanol, 4-phenyl-2-butanol, 1-phenyl-2-butanone, 4-phenyl-2-butanone, 1-phenyl-2,3-butandiol, 1-phenyl-3-hydroxy-2-butanone, 4-phenyl-3-hydroxy-2-butanone, 1-phenyl-2,3-butanedione, n-pentane, ethylphenol, ethenylphenol, 2-(4-hydroxyphenyl)ethanol, 1-(4-hydroxyphenyl)butane, 4-(4-hydroxyphenyl)-1-butene, 4-(4-hydroxyphenyl)-2-butene, 1-(4-hydroxyphenyl)-1-butene, 1-(4-hydroxyphenyl)-2-butanol, 4-(4-hydroxyphenyl)-2-butanol, 1-(4-hydroxyphenyl)-2-butanone, 4-(4-hydroxyphenyl)-2-butanone, 1-(4-hydroxyphenyl)-2,3-butandiol, 1-(4-hydroxyphenyl)-3-hydroxy-2-butanone, 4-(4-hydroxyphenyl)-3-hydroxy-2-butanone, 1-(4-hydroxyphenyl)-2,3-butanonedione, indolylethane, indolylethene, 2-(indole-3-) ethanol, n-pentane, 1-pentene, 1-pentanol, pentanal, pentanoate, 2-pentene, 2-pentanol, 3-pentanol, 2-pentanone, 3-pentanone, 4-methylpentanal, 4-methylpentanol, 2,3-pentanediol, 2-hydroxy-3-pentanone, 3-hydroxy-2-pentanone, 2,3-pentanedione, 2-methylpentane, 4-methyl-1-pentene, 4-methyl-2-pentene, 4-methyl-3-pentene, 4-methyl-2-pentanol, 2-methyl-3-pentanol, 4-methyl-2-pentanone, 2-methyl-3-pentanone, 4-methyl-2,3-pentanediol, 4-methyl-2-hydroxy-3-pentanone, 4-methyl-3-hydroxy-2-pentanone, 4-methyl-2,3-pentanedione, 1-phenylpentane, 1-phenyl-1-pentene, 1-phenyl-2-pentene, 1-phenyl-3-pentene, 1-phenyl-2-pentanol, 1-phenyl-3-pentanol, 1-phenyl-2-pentanone, 1-phenyl-3-pentanone, 1-phenyl-2,3-pentanediol, 1-phenyl-2-hydroxy-3-pentanone, 1-phenyl-3-hydroxy-2-pentanone, 1-phenyl-2,3-pentanedione, 4-methyl-1-phenylpentane, 4-methyl-1-phenyl-1-pentene, 4-methyl-1-phenyl-2-pentene, 4-methyl-1-phenyl-3-pentene, 4-methyl-1-phenyl-3-pentanol, 4-methyl-1-phenyl-2-pentanol, 4-methyl-1-phenyl-3-pentanone, 4-methyl-1-phenyl-2-pentanone, 4-methyl-1-phenyl-2,3-pentanediol, 4-methyl-1-phenyl-2,3-pentanedione, 4-methyl-1-phenyl-3-hydroxy-2-pentanone, 4-methyl-1-phenyl-2-hydroxy-3-pentanone, 1-(4-hydroxyphenyl)pentane, 1-(4-hydroxyphenyl)-1-pentene, 1-(4-hydroxyphenyl)-2-pentene, 1-(4-hydroxyphenyl)-3-pentene, 1-(4-hydroxyphenyl)-2-pentanol, 1-(4-hydroxyphenyl)-3-pentanol, 1-(4-hydroxyphenyl)-2-pentanone, 1-(4-hydroxyphenyl)-3-pentanone, 1-(4-hydroxyphenyl)-2,3-pentanediol, 1-(4-hydroxyphenyl)-2-hydroxy-3-pentanone, 1-(4-hydroxyphenyl)-3-hydroxy-2-pentanone, 1-(4-hydroxyphenyl)-2,3-pentanedione, 4-methyl-1-(4-hydroxyphenyl)pentane, 4-methyl-1-(4-hydroxyphenyl)-2-pentene, 4-methyl-1-(4-hydroxyphenyl)-3-pentene, 4-methyl-1-(4-hydroxyphenyl)-1-pentene, 4-methyl-1-(4-hydroxyphenyl)-3-pentanol, 4-methyl-1-(4-hydroxyphenyl)-2-pentanol, 4-methyl-1-(4-hydroxyphenyl)-3-pentanone, 4-methyl-1-(4-hydroxyphenyl)-2-pentanone, 4-methyl-1-(4-hydroxyphenyl)-2,3-pentanediol, 4-methyl-1-(4-hydroxyphenyl)-2,3-pentanedione, 4-methyl-1-(4-hydroxyphenyl)-3-hydroxy-2-pentanone, 4-methyl-1-(4-hydroxyphenyl)-2-hydroxy-3-pentanone, 1-indole-3-pentane, 1-(indole-3)-1-pentene, 1-(indole-3)-2-pentene, 1-(indole-3)-3-pentene, 1-(indole-3)-2-pentanol, 1-(indole-3)-3-pentanol, 1-(indole-3)-2-pentanone, 1-(indole-3)-3-pentanone, 1-(indole-3)-2,3-pentanediol, 1-(indole-3)-2-hydroxy-3-pentanone, 1-(indole-3)-3-hydroxy-2-pentanone, 1-(indole-3)-2,3-pentanedione, 4-methyl-1-(indole-3-)pentane, 4-methyl-1-(indole-3)-2-pentene, 4-methyl-1-(indole-3)-3-pentene, 4-methyl-1-(indole-3)-1-pentene, 4-methyl-2-(indole-3)-3-pentanol, 4-methyl-1-(indole-3)-2-pentanol, 4-methyl-1-(indole-3)-3-pentanone, 4-methyl-1-(indole-3)-2-pentanone, 4-methyl-1-(indole-3)-2,3-pentanediol, 4-methyl-1-(indole-3)-2,3-pentanedione, 4-methyl-1-(indole-3)-3-hydroxy-2-pentanone, 4-methyl-1-(indole-3)-2-hydroxy-3-pentanone, n-hexane, 1-hexene, 1-hexanol, hexanal, hexanoate, 2-hexene, 3-hexene, 2-hexanol, 3-hexanol, 2-hexanone, 3-hexanone, 2,3-hexanediol, 2,3-hexanedione, 3,4-hexanediol, 3,4-hexanedione, 2-hydroxy-3-hexanone, 3-hydroxy-2-hexanone, 3-hydroxy-4-hexanone, 4-hydroxy-3-hexanone, 2-methylhexane, 3-methylhexane, 2-methyl-2-hexene, 2-methyl-3-hexene, 5-methyl-1-hexene, 5-methyl-2-hexene, 4-methyl-1-hexene, 4-methyl-2-hexene, 3-methyl-3-hexene, 3-methyl-2-hexene, 3-methyl-1-hexene, 2-methyl-3-hexanol, 5-methyl-2-hexanol, 5-methyl-3-hexanol, 2-methyl-3-hexanone, 5-methyl-2-hexanone, 5-methyl-3-hexanone, 2-methyl-3,4-hexanediol, 2-methyl-3,4-hexanedione, 5-methyl-2,3-hexanediol, 5-methyl-2,3-hexanedione, 4-methyl-2,3-hexanediol, 4-methyl-2,3-hexanedione, 2-methyl-3-hydroxy-4-hexanone, 2-methyl-4-hydroxy-3-hexanone, 5-methyl-2-hydroxy-3-hexanone, 5-methyl-3-hydroxy-2-hexanone, 4-methyl-2-hydroxy-3-hexanone, 4-methyl-3-hydroxy-2-hexanone, 2,5-dimethylhexane, 2,5-dimethyl-2-hexene, 2,5-dimethyl-3-hexene, 2,5-dimethyl-3-hexanol, 2,5-dimethyl-3-hexanone, 2,5-dimethyl-3,4-hexanediol, 2,5-dimethyl-3,4-hexanedione, 2,5-dimethyl-3-hydroxy-4-hexanone, 5-methyl-1-phenylhexane, 4-methyl-1-phenylhexane, 5-methyl-1-phenyl-1-hexene, 5-methyl-1-phenyl-2-hexene, 5-methyl-1-phenyl-3-hexene, 4-methyl-1-phenyl-1-hexene, 4-methyl-1-phenyl-2-hexene, 4-methyl-1-phenyl-3-hexene, 5-methyl-1-phenyl-2-hexanol, 5-methyl-1-phenyl-3-hexanol, 4-methyl-1-phenyl-2-hexanol, 4-methyl-1-phenyl-3-hexanol, 5-methyl-1-phenyl-2-hexanone, 5-methyl-1-phenyl-3-hexanone, 4-methyl-1-phenyl-2-hexanone, 4-methyl-1-phenyl-3-hexanone, 5-methyl-1-phenyl-2,3-hexanediol, 4-methyl-1-phenyl-2,3-hexanediol, 5-methyl-1-phenyl-3-hydroxy-2-hexanone, 5-methyl-1-phenyl-2-hydroxy-3-hexanone, 4-methyl-1-phenyl-3-hydroxy-2-hexanone, 4-methyl-1-phenyl-2-hydroxy-3-hexanone, 5-methyl-1-phenyl-2,3-hexanedione, 4-methyl-1-phenyl-2,3-hexanedione, 4-methyl-1-(4-hydroxyphenyl)hexane, 5-methyl-1-(4-hydroxyphenyl)-1-hexene, 5-methyl-1-(4-hydroxyphenyl)-2-hexene, 5-methyl-1-(4-hydroxyphenyl)-3-hexene, 4-methyl-1-(4-hydroxyphenyl)-1-hexene, 4-methyl-1-(4-hydroxyphenyl)-2-hexene, 4-methyl-1-(4-hydroxyphenyl)-3-hexene, 5-methyl-1-(4-hydroxyphenyl)-2-hexanol, 5-methyl-1-(4-hydroxyphenyl)-3-hexanol, 4-methyl-1-(4-hydroxyphenyl)-2-hexanol, 4-methyl-1-(4-hydroxyphenyl)-3-hexanol, 5-methyl-1-(4-hydroxyphenyl)-2-hexanone, 5-methyl-1-(4-hydroxyphenyl)-3-hexanone, 4-methyl-1-(4-hydroxyphenyl)-2-hexanone, 4-methyl-1-(4-hydroxyphenyl)-3-hexanone, 5-methyl-1-(4-hydroxyphenyl)-2,3-hexanediol, 4-methyl-1-(4-hydroxyphenyl)-2,3-hexanediol, 5-methyl-1-(4-hydroxyphenyl)-3-hydroxy-2-hexanone, 5-methyl-1-(4-hydroxyphenyl)-2-hydroxy-3-hexanone, 4-methyl-1-(4-hydroxyphenyl)-3-hydroxy-2-hexanone, 4-methyl-1-(4-hydroxyphenyl)-2-hydroxy-3-hexanone, 5-methyl-1-(4-hydroxyphenyl)-2,3-hexanedione, 4-methyl-1-(4-hydroxyphenyl)-2,3-hexanedione, 4-methyl-1-(indole-3-)hexane, 5-methyl-1-(indole-3)-1-hexene, 5-methyl-1-(indole-3)-2-hexene, 5-methyl-1-(indole-3)-3-hexene, 4-methyl-1-(indole-3)-1-hexene, 4-methyl-1-(indole-3)-2-hexene, 4-methyl-1-(indole-3)-3-hexene, 5-methyl-1-(indole-3)-2-hexanol, 5-methyl-1-(indole-3)-3-hexanol, 4-methyl-1-(indole-3)-2-hexanol, 4-methyl-1-(indole-3)-3-hexanol, 5-methyl-1-(indole-3)-2-hexanone, 5-methyl-1-(indole-3)-3-hexanone, 4-methyl-1-(indole-3)-2-hexanone, 4-methyl-1-(indole-3)-3-hexanone, 5-methyl-1-(indole-3)-2,3-hexanediol, 4-methyl-1-(indole-3)-2,3-hexanediol, 5-methyl-1-(indole-3)-3-hydroxy-2-hexanone, 5-methyl-1-(indole-3)-2-hydroxy-3-hexanone, 4-methyl-1-(indole-3)-3-hydroxy-2-hexanone, 4-methyl-1-(indole-3)-2-hydroxy-3-hexanone, 5-methyl-1-(indole-3)-2,3-hexanedione, 4-methyl-1-(indole-3)-2,3-hexanedione, n-heptane, 1-heptene, 1-heptanol, heptanal, heptanoate, 2-heptene, 3-heptene, 2-heptanol, 3-heptanol, 4-heptanol, 2-heptanone, 3-heptanone, 4-heptanone, 2,3-heptanediol, 2,3-heptanedione, 3,4-heptanediol, 3,4-heptanedione, 2-hydroxy-3-heptanone, 3-hydroxy-2-heptanone, 3-hydroxy-4-heptanone, 4-hydroxy-3-heptanone, 2-methylheptane, 3-methylheptane, 6-methyl-2-heptene, 6-methyl-3-heptene, 2-methyl-3-heptene, 2-methyl-2-heptene, 5-methyl-2-heptene, 5-methyl-3-heptene, 3-methyl-3-heptene, 2-methyl-3-heptanol, 2-methyl-4-heptanol, 6-methyl-3-heptanol, 5-methyl-3-heptanol, 3-methyl-4-heptanol, 2-methyl-3-heptanone, 2-methyl-4-heptanone, 6-methyl-3-heptanone, 5-methyl-3-heptanone, 3-methyl-4-heptanone, 2-methyl-3,4-heptanediol, 2-methyl-3,4-heptanedione, 6-methyl-3,4-heptanediol, 6-methyl-3,4-heptanedione, 5-methyl-3,4-heptanediol, 5-methyl-3,4-heptanedione, 2-methyl-3-hydroxy-4-heptanone, 2-methyl-4-hydroxy-3-heptanone, 6-methyl-3-hydroxy-4-heptanone, 6-methyl-4-hydroxy-3-heptanone, 5-methyl-3-hydroxy-4-heptanone, 5-methyl-4-hydroxy-3-heptanone, 2,6-dimethylheptane, 2,5-dimethylheptane, 2,6-dimethyl-2-heptene, 2,6-dimethyl-3-heptene, 2,5-dimethyl-2-heptene, 2,5-dimethyl-3-heptene, 3,6-dimethyl-3-heptene, 2,6-dimethyl-3-heptanol, 2,6-dimethyl-4-heptanol, 2,5-dimethyl-3-heptanol, 2,5-dimethyl-4-heptanol, 2,6-dimethyl-3,4-heptanediol, 2,6-dimethyl-3,4-heptanedione, 2,5-dimethyl-3,4-heptanediol, 2,5-dimethyl-3,4-heptanedione, 2,6-dimethyl-3-hydroxy-4-heptanone, 2,6-dimethyl-4-hydroxy-3-heptanone, 2,5-dimethyl-3-hydroxy-4-heptanone, 2,5-dimethyl-4-hydroxy-3-heptanone, n-octane, 1-octene, 2-octene, 1-octanol, octanal, octanoate, 3-octene, 4-octene, 4-octanol, 4-octanone, 4,5-octanediol, 4,5-octanedione, 4-hydroxy-5-octanone, 2-methyloctane, 2-methyl-3-octene, 2-methyl-4-octene, 7-methyl-3-octene, 3-methyl-3-octene, 3-methyl-4-octene, 6-methyl-3-octene, 2-methyl-4-octanol, 7-methyl-4-octanol, 3-methyl-4-octanol, 6-methyl-4-octanol, 2-methyl-4-octanone, 7-methyl-4-octanone, 3-methyl-4-octanone, 6-methyl-4-octanone, 2-methyl-4,5-octanediol, 2-methyl-4,5-octanedione, 3-methyl-4,5-octanediol, 3-methyl-4,5-octanedione, 2-methyl-4-hydroxy-5-octanone, 2-methyl-5-hydroxy-4-octanone, 3-methyl-4-hydroxy-5-octanone, 3-methyl-5-hydroxy-4-octanone, 2,7-dimethyloctane, 2,7-dimethyl-3-octene, 2,7-dimethyl-4-octene, 2,7-dimethyl-4-octanol, 2,7-dimethyl-4-octanone, 2,7-dimethyl-4,5-octanediol, 2,7-dimethyl-4,5-octanedione, 2,7-dimethyl-4-hydroxy-5-octanone, 2,6-dimethyloctane, 2,6-dimethyl-3-octene, 2,6-dimethyl-4-octene, 3,7-dimethyl-3-octene, 2,6-dimethyl-4-octanol, 3,7-dimethyl-4-octanol, 2,6-dimethyl-4-octanone, 3,7-dimethyl-4-octanone, 2,6-dimethyl-4,5-octanediol, 2,6-dimethyl-4,5-octanedione, 2,6-dimethyl-4-hydroxy-5-octanone, 2,6-dimethyl-5-hydroxy-4-octanone, 3,6-dimethyloctane, 3,6-dimethyl-3-octene, 3,6-dimethyl-4-octene, 3,6-dimethyl-4-octanol, 3,6-dimethyl-4-octanone, 3,6-dimethyl-4,5-octanediol, 3,6-dimethyl-4,5-octanedione, 3,6-dimethyl-4-hydroxy-5-octanone, n-nonane, 1-nonene, 1-nonanol, nonanal, nonanoate, 2-methylnonane, 2-methyl-4-nonene, 2-methyl-5-nonene, 8-methyl-4-nonene, 2-methyl-5-nonanol, 8-methyl-4-nonanol, 2-methyl-5-nonanone, 8-methyl-4-nonanone, 8-methyl-4,5-nonanediol, 8-methyl-4,5-nonanedione, 8-methyl-4-hydroxy-5-nonanone, 8-methyl-5-hydroxy-4-nonanone, 2,8-dimethylnonane, 2,8-dimethyl-3-nonene, 2,8-dimethyl-4-nonene, 2,8-dimethyl-5-nonene, 2,8-dimethyl-4-nonanol, 2,8-dimethyl-5-nonanol, 2,8-dimethyl-4-nonanone, 2,8-dimethyl-5-nonanone, 2,8-dimethyl-4,5-nonanediol, 2,8-dimethyl-4,5-nonanedione, 2,8-dimethyl-4-hydroxy-5-nonanone, 2,8-dimethyl-5-hydroxy-4-nonanone, 2,7-dimethylnonane, 3,8-dimethyl-3-nonene, 3,8-dimethyl-4-nonene, 3,8-dimethyl-5-nonene, 3,8-dimethyl-4-nonanol, 3,8-dimethyl-5-nonanol, 3,8-dimethyl-4-nonanone, 3,8-dimethyl-5-nonanone, 3,8-dimethyl-4,5-nonanediol, 3,8-dimethyl-4,5-nonanedione, 3,8-dimethyl-4-hydroxy-5-nonanone, 3,8-dimethyl-5-hydroxy-4-nonanone, n-decane, 1-decene, 1-decanol, decanoate, 2,9-dimethyldecane, 2,9-dimethyl-3-decene, 2,9-dimethyl-4-decene, 2,9-dimethyl-5-decanol, 2,9-dimethyl-5-decanone, 2,9-dimethyl-5,6-decanediol, 2,9-dimethyl-6-hydroxy-5-decanone, 2,9-dimethyl-5,6-decanedionen-undecane, 1-undecene, 1-undecanol, undecanal. undecanoate, n-dodecane, 1-dodecene, 1-dodecanol, dodecanal, dodecanoate, n-dodecane, 1-decadecene, 1-dodecanol, ddodecanal, dodecanoate, n-tridecane, 1-tridecene, 1-tridecanol, tridecanal, tridecanoate, n-tetradecane, 1-tetradecene, 1-tetradecanol, tetradecanal, tetradecanoate, n-pentadecane, 1-pentadecene, 1-pentadecanol, pentadecanal, pentadecanoate, n-hexadecane, 1-hexadecene, 1-hexadecanol, hexadecanal, hexadecanoate, n-heptadecane, 1-heptadecene, 1-heptadecanol, heptadecanal, heptadecanoate, n-octadecane, 1-octadecene, 1-octadecanol, octadecanal, octadecanoate, n-nonadecane, 1-nonadecene, 1-nonadecanol, nonadecanal, nonadecanoate, eicosane, 1-eicosene, 1-eicosanol, eicosanal, eicosanoate, 3-hydroxy propanal, 1,3-propanediol, 4-hydroxybutanal, 1,4-butanediol, 3-hydroxy-2-butanone, 2,3-butandiol, 1,5-pentane diol, homocitrate, homoisocitorate, b-hydroxy adipate, glutarate, glutarsemialdehyde, glutaraldehyde, 2-hydroxy-1-cyclopentanone, 1,2-cyclopentanediol, cyclopentanone, cyclopentanol, (S)-2-acetolactate, (R)-2,3-Dihydroxy-isovalerate, 2-oxoisovalerate, isobutyryl-CoA, isobutyrate, isobutyraldehyde, 5-amino pentaldehyde, 1,10-diaminodecane, 1,10-diamino-5-decene, 1,10-diamino-5-hydroxydecane, 1,10-diamino-5-decanone, 1,10-diamino-5,6-decanediol, 1,10-diamino-6-hydroxy-5-decanone, phenylacetoaldehyde, 1,4-diphenylbutane, 1,4-diphenyl-1-butene, 1,4-diphenyl-2-butene, 1,4-diphenyl-2-butanol, 1,4-diphenyl-2-butanone, 1,4-diphenyl-2,3-butanediol, 1,4-diphenyl-3-hydroxy-2-butanone, 1-(4-hydeoxyphenyl)-4-phenylbutane, 1-(4-hydeoxyphenyl)-4-phenyl-1-butene, 1-(4-hydeoxyphenyl)-4-phenyl-2-butene, 1-(4-hydeoxyphenyl)-4-phenyl-2-butanol, 1-(4-hydeoxyphenyl)-4-phenyl-2-butanone, 1-(4-hydeoxyphenyl)-4-phenyl-2,3-butanediol, 1-(4-hydeoxyphenyl)-4-phenyl-3-hydroxy-2-butanone, 1-(indole-3)-4-phenylbutane, 1-(indole-3)-4-phenyl-1-butene, 1-(indole-3)-4-phenyl-2-butene, 1-(indole-3)-4-phenyl-2-butanol, 1-(indole-3)-4-phenyl-2-butanone, 1-(indole-3)-4-phenyl-2,3-butanediol, 1-(indole-3)-4-phenyl-3-hydroxy-2-butanone, 4-hydroxyphenylacetoaldehyde, 1,4-di(4-hydroxyphenyl)butane, 1,4-di(4-hydroxyphenyl)-1-butene, 1,4-di(4-hydroxyphenyl)-2-butene, 1,4-di(4-hydroxyphenyl)-2-butanol, 1,4-di(4-hydroxyphenyl)-2-butanone, 1,4-di(4-hydroxyphenyl)-2,3-butanediol, 1,4-di(4-hydroxyphenyl)-3-hydroxy-2-butanone, 1-(4-hydroxyphenyl)-4-(indole-3-)butane, 1-(4-hydroxyphenyl)-4-(indole-3)-1-butene, 1-di(4-hydroxyphenyl)-4-(indole-3)-2-butene, 1-(4-hydroxyphenyl)-4-(indole-3)-2-butanol, 1-(4-hydroxyphenyl)-4-(indole-3)-2-butanone, 1-(4-hydroxyphenyl)-4-(indole-3)-2,3-butanediol, 1-(4-hydroxyphenyl-4-(indole-3)-3-hydroxy-2-butanone, indole-3-acetoaldehyde, 1,4-di(indole-3-)butane, 1,4-di(indole-3)-1-butene, 1,4-di(indole-3)-2-butene, 1,4-di(indole-3)-2-butanol, 1,4-di(indole-3)-2-butanone, 1,4-di(indole-3)-2,3-butanediol, 1,4-di(indole-3)-3-hydroxy-2-butanone, succinate semialdehyde, hexane-1,8-dicarboxylic acid, 3-hexene-1,8-dicarboxylic acid, 3-hydroxy-hexane-1,8-dicarboxylic acid, 3-hexanone-1,8-dicarboxylic acid, 3,4-hexanediol-1,8-dicarboxylic acid, 4-hydroxy-3-hexanone-1,8-dicarboxylic acid, fucoidan, chlorophyll, carotenoid, and the like.

“Stress conditions” refer to any condition or combination thereof that imposes a stress upon the Cyanobacteria, including environmental, physical, and/or genetic stresses, and which reduces biomass accumulation, cell division, or both. Under stress conditions, biomass accumulation and/or cell division of a photosynthetic microorganism can be reduced by at least about 10%, 15%, 20%, 25%, 30%, 35%, 40%, 45%, 50%, 55%, 60%, 65%, 70%, 75%, 80%, 85%, 90%, 95%, 98%, 99%, or 100% relative to a corresponding photosynthetic microorganism grown under non-stress conditions.

Examples of stresses include but are not limited to: reduced or increased temperature as compared to standard; nutrient deprivation, e.g., reduced levels or absence of one or more essential nutrients such as nitrogen, sulfur, phosphorous, and/or phosphate; reduced or increased CO₂ levels as compared to a standard; reduced or increased light exposure, e.g., intensity or duration, as compared to standard; exposure to reduced or increased nitrogen, iron, sulfur, phosphorus, and/or copper as compared to standard; altered pH, e.g., more or less acidic or basic, as compared to standard; altered salt conditions as compared to standard; exposure to an agent that causes DNA synthesis inhibitor or protein synthesis inhibition; increased or decreased culture density as compared to standard; introduced or overexpressed polynucleotides encoding one or more polypeptides associated with reduction of biomass accumulation, cell division, or division as compared to a wild-type photosynthetic microorganism; and altered expression of one or more polypeptides associated with reduction of biomass accumulation, cell division, or division as compared to a wild-type photosynthetic microorganism. Standard growth and culture conditions (e.g., non-stress conditions) for various Cyanobacteria are known in the art.

“Reduced nitrogen conditions,” or conditions of “nitrogen limitation,” refer generally to culture conditions in which a certain fraction or percentage of a standard nitrogen concentration is present in the culture media. Such fractions typically include, but are not limited to, about 1/50, 1/40, 1/30, 1/10, ⅕, ¼, or about ½ the standard nitrogen conditions. Such percentages typically include, but are not limited to, less than about 1%, 2%, 3%, 4%, 5%, 6%, 7%, 8%, 9%, 10%, 15%, 20%, 30%, 40%, or 50% the standard nitrogen conditions. “Standard” nitrogen conditions can be estimated, for example, by the amount of nitrogen present in BG11 media, as exemplified herein and known in the art. For instance, BG11 media usually contains nitrogen in the form of NaNO₃ at a concentration of about 1.5 grams/liter (see, e.g., Rippka et al., J. Gen Microbiol. 111:1-61, 1979).

The term “maintenance of photosynthetic activity” under stress conditions includes, for instance, where photosynthetic activity of a modified photosynthetic microorganism (that accumulates a reduced amount of glycogen as compared to the wild-type photosynthetic microorganism) under a given stress condition is substantially greater than photosynthetic activity of a corresponding wild-type photosynthetic microorganism (e.g., of the same genus/species) under the same or comparable stress condition. Also included is where the photosynthetic activity of a modified photosynthetic microorganism (that accumulates a reduced amount of glycogen as compared to the wild-type photosynthetic microorganism) under a given stress condition is at least about 20% of its photosynthetic activity under non-stress conditions. In these and related embodiments, the photosynthetic activity (for comparison) can be measured, for example, at about day 2, 3, 4, 5, 6, 7, 8, 9, 10, 11, 12, 13, 14, 15, 16, 17, 18, 19, 20, or 21 post-initiation or re-initiation of the stress condition, including all ranges in between.

By “obtained from” is meant that a sample such as, for example, a polynucleotide or polypeptide is isolated from, or derived from, a particular source, such as a desired organism or a specific tissue within a desired organism. “Obtained from” can also refer to the situation in which a polynucleotide or polypeptide sequence is isolated from, or derived from, a particular organism or tissue within an organism. For example, a polynucleotide sequence encoding a reference polypeptide described herein may be isolated from a variety of prokaryotic or eukaryotic organisms, or from particular tissues or cells within certain eukaryotic organism.

The term “operably linked” as used herein means placing a gene under the regulatory control of a promoter, which then controls the transcription and optionally the translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e., the gene from which it is derived. “Constitutive promoters” are typically active, i.e., promote transcription, under most conditions. “Inducible promoters” are typically active only under certain conditions, such as in the presence of a given molecule factor (e.g., IPTG) or a given environmental condition (e.g., particular CO₂ concentration, nutrient levels, light, heat). In the absence of that condition, inducible promoters typically do not allow significant or measurable levels of transcriptional activity. For example, inducible promoters may be induced according to temperature, pH, a hormone, a metabolite (e.g., lactose, mannitol, an amino acid), light (e.g., wavelength specific), osmotic potential (e.g., salt induced), a heavy metal, or an antibiotic. Numerous standard inducible promoters will be known to one of skill in the art.

The recitation “polynucleotide” or “nucleic acid” as used herein designates mRNA, RNA, cRNA, rRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA and RNA.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide, or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs that encode these enzymes.

With regard to polynucleotides, the term “exogenous” refers to a polynucleotide sequence that does not naturally-occur in a wild-type cell or organism, but is typically introduced into the cell by molecular biological techniques. Examples of exogenous polynucleotides include vectors, plasmids, and/or man-made nucleic acid constructs encoding a desired protein. With regard to polynucleotides, the term “endogenous” or “native” refers to naturally-occurring polynucleotide sequences that may be found in a given wild-type cell or organism. For example, certain Cyanobacterial species do not typically contain a DGAT gene, and, therefore, do not comprise an “endogenous” polynucleotide sequence that encodes a DGAT polypeptide. Also, a particular polynucleotide sequence that is isolated from a first organism and transferred to second organism by molecular biological techniques is typically considered an “exogenous” polynucleotide with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The recitations “mutation” or “deletion,” in relation to the genes of a “glycogen biosynthesis or storage pathway” or certain “lipid biosynthesis proteins,” refer generally to those changes or alterations in a photosynthetic microorganism, e.g., a Cyanobacterium, that render the product of that gene non-functional or having reduced function with respect to the synthesis and/or storage of glycogen or biosynthesis of a given lipid. Examples of such changes or alterations include nucleotide substitutions, deletions, or additions to the coding or regulatory sequences of a targeted gene (e.g., glgA, glgC, pgm, aldehyde dehydrogenase, aldehyde decarbonylase, Aas), in whole or in part, which disrupt, eliminate, down-regulate, or significantly reduce the expression of the polypeptide encoded by that gene, whether at the level of transcription or translation, and/or which produce a relatively inactive (e.g., mutated or truncated) or unstable polypeptide. Techniques for producing such alterations or changes, such as by recombination with a vector having a selectable marker, are exemplified herein and known in the molecular biological art. In particular embodiments, one or more alleles of a gene, e.g., two or all alleles, may be mutated or deleted within a photosynthetic microorganism. In particular embodiments, modified photosynthetic microorganisms, e.g., Cyanobacteria, of the present invention are merodiploids or partial diploids.

The “deletion” of a targeted gene may also be accomplished by targeting the mRNA of that gene, such as by using various antisense technologies (e.g., antisense oligonucleotides and siRNA) known in the art. Accordingly, targeted genes may be considered “non-functional” when the polypeptide or enzyme encoded by that gene is not expressed by the modified photosynthetic microorganism, or is expressed in negligible amounts, such that the modified photosynthetic microorganism produces or accumulates less of the polypeptide or enzyme product (e.g., glycogen or glycogen precursor or related molecules—see FIG. 21) than an unmodified or differently modified photosynthetic microorganism.

In certain aspects, a targeted gene may be rendered “non-functional” by changes or mutations at the nucleotide level that alter the amino acid sequence of the encoded polypeptide, such that a modified polypeptide is expressed, but which has reduced function or activity with respect to its enzymatic activity (e.g., glycogen synthesis, glycogen storage, lipid biosynthesis), whether by modifying that polypeptide's active site, its cellular localization, its stability, or other functional features apparent to a person skilled in the art. Such modifications to the coding sequence of a polypeptide involved in glycogen biosynthesis or storage may be accomplished according to known techniques in the art, such as site directed mutagenesis at the genomic level and/or natural selection (i.e., directed evolution) of a given photosynthetic microorganism.

“Polypeptide,” “polypeptide fragment,” “peptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions.

The recitation polypeptide “variant” refers to polypeptides that are distinguished from a reference polypeptide sequence by the addition, deletion or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

The term “reference sequence” refers generally to a nucleic acid coding sequence, or amino acid sequence, to which another sequence is being compared. All polypeptide and polynucleotide sequences described herein are included as references sequences, including those described by name (e.g., glycogen synthesis and/or storage genes/proteins, lipid biosynthesis genes/proteins, glycogen breakdown genes/proteins, orf1593, orf0489, orf1594, TesA, ACP, aDGAT) and those described in the Sequence Listing.

The present invention contemplates the use in the methods described herein of variants of full-length enzymes or reference sequences, for instance, those having acyl-ACP reductase activity, ACP activity, glycogen breakdown activity, diacylglyecerol transferase activity (DGAT), fatty acyl-CoA synthetase activity, aldehyde dehydrogenase activity, alcohol dehydrogenase activity, and/or acetyl-CoA carboxylase activity, among other reference sequences described herein, truncated fragments of these full-length enzymes and polypeptides, variants of truncated fragments, as well as their related biologically active fragments. Typically, biologically active fragments of a polypeptide may participate in an interaction, for example, an intra-molecular or an inter-molecular interaction. An inter-molecular interaction can be a specific binding interaction or an enzymatic interaction (e.g., the interaction can be transient and a covalent bond is formed or broken).

Biologically active fragments of a polypeptide/enzyme having a selected activity include peptides comprising amino acid sequences sufficiently similar to, or derived from, the amino acid sequences of a (putative) full-length reference polypeptide sequence. Typically, biologically active fragments comprise a domain or motif with at least one activity of a reference sequence or enzyme described herein, such as an acyl-ACP reductase, aldehyde decarbonylase, aldehyde dehydrogenase, alcohol dehydrogenase, ACP polypeptide, DGAT polypeptide, fatty acyl-CoA synthetase polypeptide, acetyl-CoA carboxylase polypeptide, or a polypeptide associated with a glycogen breakdown pathway, and may include one or more (and in some cases all) of the various active domains. A biologically active fragment of such polypeptides can be a polypeptide fragment which is, for example, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 220, 240, 260, 280, 300, 320, 340, 360, 380, 400, 450, 500, 600 or more contiguous amino acids, including all integers in between, of a reference polypeptide sequence. In certain embodiments, a biologically active fragment comprises a conserved enzymatic sequence, domain, or motif, as described elsewhere herein and known in the art. Suitably, the biologically-active fragment has no less than about 1%, 10%, 25%, 50% of an activity of the wild-type polypeptide from which it is derived.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 97%, 98%, 99% or 100% sequence identity to any of the reference sequences described herein (see, e.g., Sequence Listing), typically where the polypeptide variant maintains at least one biological activity of the reference polypeptide.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

As used herein, the term “triglyceride” (triacylglycerol or neutral fat) refers to a fatty acid triester of glycerol. Triglycerides are typically non-polar and water-insoluble.

“Phosphoglycerides” (or glycerophospholipids) are major lipid components of biological membranes, and include, for example, any derivative of sn-glycero-3-phosphoric acid that contains at least one O-acyl, or O-alkyl or O-alk-1′-enyl residue attached to the glycerol moiety and a polar head made of a nitrogenous base, a glycerol, or an inositol unit. Phosphoglycerides can also be characterized as amphipathic lipids formed by esters of acylglycerols with phosphate and another hydroxylated compound.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95%, 96%, 97%, 98%, 99% or greater of some given quantity.

“Transformation” refers to the permanent, heritable alteration in a cell resulting from the uptake and incorporation of foreign DNA into the host-cell genome; also, the transfer of an exogenous gene from one organism into the genome of another organism.

By “vector” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated. Such a vector may comprise specific sequences that allow recombination into a particular, desired site of the host chromosome. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably one which is operably functional in a photosynthetic microorganism cell, such as a Cyanobacterial cell. The vector can include a reporter gene, such as a green fluorescent protein (GFP), which can be either fused in frame to one or more of the encoded polypeptides, or expressed separately. The vector can also include a selection marker such as an antibiotic resistance gene that can be used for selection of suitable transformants.

The term “wild-type” refers to a gene or gene product that has the characteristics of that gene or gene product when isolated from a naturally-occurring source. A wild-type gene or gene product (e.g., a polypeptide) is that which is most frequently observed in a population and is thus arbitrarily designed the “normal” or “wild-type” form of the gene.

### Continuous Production Systems

The present invention relates, in part, to the discovery that modified photosynthetic organisms having reduced glycogen accumulation, e.g., due to reduced expression of one or more genes involved in glycogen biosynthesis, are able to undergo and maintain photosynthesis and produce carbon-containing compounds even when grown under stress conditions that reduce their growth. Thus, such modified photosynthetic organisms may be utilized as a continuous production system for carbon-containing compounds. In certain embodiments, such products are secreted, whereas in other embodiments, they may accumulate intracellulary in the modified photosynthetic organism. Therefore, carbon-containing compounds may be harvested from either the media or the organisms, respectively.

As described herein, the present invention provides for a system for producing a carbon-containing compound, which comprises both a modified photosynthetic organism (e.g., microorganism) that accumulates a reduced amount of glycogen as compared to the wild-type photosynthetic microorganism and a culture system for culturing said modified photosynthetic microorganism under a stress condition, wherein said modified photosynthetic organism maintains photosynthetic activity and accumulates reduced biomass when grown under said stress condition as compared to when grown under non-stress conditions.

In a related embodiment, the present invention also includes a method for producing a carbon-containing compound other than glycogen, comprising culturing in a culture media a modified photosynthetic organism that accumulates a reduced amount of glycogen as compared to the wild-type photosynthetic organism under a stress condition, wherein said modified photosynthetic microorganism maintains photosynthetic activity and accumulates reduced biomass when grown under said stress condition as compared to when grown under non-stress conditions.

Modified photosynthetic organisms that accumulate a reduced amount of glycogen and may be used according to the present invention are described in further detail below.

While a variety of stress conditions, including those defined herein may be used, in particular embodiments, the stress condition is a reduced amount of an essential nutrient, such as, e.g., nitrogen, sulfur, phosphate or phosphorous. In particular embodiments, the level of the nutrient is less than or equal to 50%, less than or equal to 40%, less than or equal to 30%, less than or equal to 20%, less than or equal to 10%, less than or equal to 5%, or less than or equal to 1% of the amount of nutrient considered standard for growth of the particular microorganism.

In certain embodiments, the system provides or the method comprises culturing the modified photosynthetic organism under conditions that provide a stress condition at a level that uncouples photosynthesis from growth, but also maintains the culture at an optical density advantageous for the organism's use of sunlight in photosynthesis. In various embodiments, this end is achieved by continually providing the stress condition (e.g., reduced nutrient) over a duration of time and at a level sufficient to inhibit cell growth while permitting photosynthesis. For example, where the stress condition is a reduced level of a nutrient, a particular reduced level may be maintained for a period of time. In particular embodiments, the duration of time is: from 1 day to 1 year, from 1 day to 6 months, from 1 day to 1 month, or from 1 day to 1 week, or at least about or up to about 1, 2, 3, 4, 5, 7, 8, 9, 10, 11, 12, 13, or 14 days, weeks or months. In certain embodiments, the level of the stress condition, e.g., the amount of nutrient such as nitrogen, is maintained at a concentration that significantly reduces prevents growth of both the wild-type and the modified photosynthetic microorganism, while triggering photosystem degradation in the wild-type microorganism but not the modified microorganism. In particular embodiments, a system of the invention may thus provide for continuous or pulsed delivery of a nutrient to a culture of modified photosynthetic organisms used according to the present invention.

In these and related embodiments, the stress condition may be relieved or removed at one or more times (e.g., by providing one or more pulses of a reduced essential nutrient), at a frequency and in an amount sufficient to maintain the culture at an optical density optimal for the photosynthetic use of sunlight or which prevents growth of both the wild-type and the modified photosynthetic organism, while triggering photosystem degradation in the wild-type microorganism but not the modified microorganism. For instance, in certain aspects the stress condition can be relieved when the ratio of absorbance of the culture at 680/750 nm is (or falls to) about 10%-90% of the ratio of a corresponding culture (e.g., of the same or comparable modified photosynthetic microorganism) under non-stress conditions, including where the ratio is or falls to about 10%, 15%, 20%, 30%, 35%, 40%, 50%, 55%, 60%, 65%, 70%, 75%, 80%, 85%, 90%, 10%-20%, 20%-30%, 30%-40%, 40%-50%, 60%-70%, 80%-90%, 10%-30%, 20%-40%, 30%-50%, 40%-60%, 50%-70%, 60%-80%, 70%-90%, 10%-40%, 20%-50%, 30%-60%, 40%-70%, 50%-80%, 60%-90%, 10%-50%, 20%-60%, 30%-70%, 40%-80%, or 50%-90% of the ratio of a corresponding culture under non-stress conditions, where non-stress conditions optionally comprise nutrient replete conditions. Separately or in combination with such determinations, the stress condition can be relieved on a periodic basis, for instance, at about 1, 2, 3, 4, 5, 6, 7, 8, 9, 10, 11, 12, 13, 14, 15, 16, 17, 18, 19, 20, 21, 22, 23, 24, 25, 26, 27, 28 or more days following initiation of the stress condition.

In certain instances, relieving the stress condition increases photosynthetic activity of the modified photosynthetic microorganism and/or increases the ratio of absorbance of the culture. For example, in some aspects, relieving the stress condition (e.g., pulsing with an otherwise reduced essential nutrient) increases photosynthetic activity by at least about 5%, 10%, 15%, 20%, 30%, 35%, 40%, 50%, 55%, 60%, 65%, 70%, 75%, 80%, 85%, 90%, 100%, 200%, 300%, 400%, 500% or more relative to photosynthetic activity immediately prior to relief of said stress condition. Photosynthetic activity can be measured, for example, by CO₂ fixation and/or chlorophyll levels, as described herein. In many instances, the modified photosynthetic microorganism maintains the increased photosynthetic activity for a substantially longer time (e.g., about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 times longer) than a wild-type photosynthetic microorganism under the same or comparable culture conditions. In certain instances, for example, following relief of the stress condition and increased photosynthetic activity, the subsequent decrease in photosynthetic activity by the modified photosynthetic microorganism is substantially less (e.g., about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 times less) than the subsequent decrease in photosynthetic activity by a wild-type photosynthetic microorganism culture under the same or comparable culture conditions.

In some embodiments, following removal of or relief from the stress condition, the ratio of absorbance increases (e.g., temporarily increases) to greater than about 90%, 91%, 92%, 93%, 94%, 95%, 96%, 98%, 99%, 100%, 105%, 110% or more of the ratio of a corresponding culture (e.g., of the same or comparable modified photosynthetic microorganism) under non-stress conditions, where non-stress conditions optionally comprise nutrient replete conditions. In most instances, the modified photosynthetic microorganism culture maintains this increased ratio of absorbance for a substantially longer time (e.g., about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 times longer) than a wild-type photosynthetic microorganism culture under the same or comparable culture conditions.

In specific aspects, the stress condition comprises reduced level of an essential nutrient, and relieving the stress condition comprises adding (i.e., pulsing the culture with) the essential nutrient in an amount sufficient to increase photosynthetic activity of the modified photosynthetic microorganism and/or increase the ratio of absorbance of the culture. In particular embodiments, the essential nutrient is selected from at least one of nitrogen, sulfur, and phosphorous. In specific embodiments, the essentially nutrient is nitrogen, which can be added to the culture in the form of NaNO₃, NH₄Cl, (NH₄)₂SO₄, NH₄HCO₃, CH₄N₂O, KNO₃, or any combination thereof, optionally to achieve a final concentration ranging from about 0.02 mM to about 1 mM to about 10 mM to about 20 mM to about 30 mM or to about 40 mM.

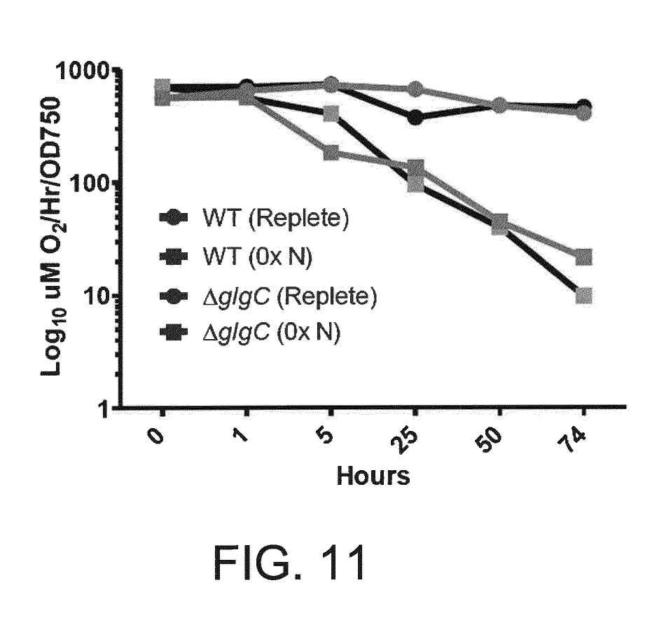

In any of these embodiments, the methods can further comprise repeating the step of relieving the stress condition, for instance, periodically and/or when certain cell culture conditions are observed. In practice, such repetition can occur almost indefinitely, as desired. In certain aspects, the step of relieving the stress condition can be repeated about every 2, 3, 4, 5, 6, 7, 8, 9, 10, 11, 12, 13, 14, 15, 16, 17, 18, 19, 20, 21, 22, 23, 24, 25, 26, 27, or 28 days following previous relief of the stress condition. Alternatively or in combination with periodic relief of the stress condition, the stress condition can be relieved any time the ratio of absorbance of the culture is or falls to about 10%-90% of the ratio of a corresponding culture under non-stress conditions, including any time the ratio is or falls to about 10%, 15%, 20%, 30%, 35%, 40%, 50%, 55%, 60%, 65%, 70%, 75%, 80%, 85%, 90%, 10%-20%, 20%-30%, 30%-40%, 40%-50%, 60%-70%, 80%-90%, 10%-30%, 20%-40%, 30%-50%, 40%-60%, 50%-70%, 60%-80%, 70%-90%, 10%-40%, 20%-50%, 30%-60%, 40%-70%, 50%-80%, 60%-90%, 10%-50%, 20%-60%, 30%-70%, 40%-80%, or 50%-90% of the ratio of a corresponding culture under non-stress conditions. Periodic or occasional relief of the stress condition, for instance, by pulsing with an otherwise reduced essential nutrient, can prolong the window during which the modified photosynthetic microorganisms described herein (e.g., reduced glycogen mutants) are able to maintain the optimal combination of photosynthetic activity and reduced cell growth/biomass accumulation. Because such modified photosynthetic microorganisms have a much larger window than wild-type for this optimal combination (see FIG. 11), it is not only easier to prolong this window (e.g., indefinitely) in such microorganisms, but also less expensive because of the reduced need for otherwise costly nutrients.

In certain embodiments, the culture is maintained at an optical cell density ranging from 0.25-2.0, 0.5-1.5, or about 1.0, i.e., within 10% of 1.0. In certain embodiments, this optical density is maintained for at least 50%, at least 75%, or at least 90% of the duration of the time that the culture is maintained under stress conditions. In one embodiment, the optimal photosynthetic rate is the rate of carbon fixation and reductant (e.g., NADPH) generation that maximizes production of the carbon-containing compound without loss of photosynthetic capacity over a defined time period, which may be, e.g., 1 day, 2 days, 1 week, 2 weeks or 1 month, 2 months, or 3 months.

In certain embodiments, the modified photosynthetic organism intracellularly accumulates and/or secretes an increased amount of a carbon-containing compound, such as a specialty chemical or a precursor or intermediate thereof, under said stress condition as compared to the wild-type microorganism. In specific embodiments, the amount of one or more of such carbon-containing compounds produced or secreted by the modified photosynthetic microorganism when grown at about 50-100 uE of light is at least about 10%, at least 15%, at least about 20%, at least about 25%, or at least about 30% of its dry weight per day.

In a specific embodiment, the present invention includes a production system or method for maintaining photosynthesis while reducing growth by reducing intracellular levels of 2-oxoglutarate in a photosynthetic organism.

In yet another embodiment, the present invention includes a method for increasing the secretion of glucose by a modified photosynthetic organism, which includes introducing or expressing (e.g., overexpressing) a polynucleotide encoding a polypeptide associated with glucose permeability, optionally under the control of an inducible promoter, in a modified photosynthetic organism having reduced glycogen accumulations, including any of those described herein, and then growing the modified photosynthetic microorganism under stress conditions. Without wishing to be bound by theory, it is understood that internal glucose levels are increased under stress, e.g., nitrogen stress, in the glycogen mutant, as compared to the wild-type under the same condition, so the glucose secretion triggered by expression of the polypeptide associated with glucose permeability renders glucose a carbon and reductant sink. Examples of various polypeptides associated with glucose permeability include, but are not limited to the glucose permease and glucose/H+ symporters described herein.

In particular embodiments of the various systems and methods of the present invention, the modified photosynthetic microorganism having reduced glycogen accumulation “maintains photosynthetic activity” under stress conditions of at least 20%, at least 30%, at least 40%, at least 50%, at least 60%, at least 70%, at least 80%, or at least 90% of its photosynthetic activity under non-stress conditions. In some aspects, the modified photosynthetic microorganism having reduced glycogen accumulation “maintains photosynthetic activity” under stress conditions by having photosynthetic activity that is substantially greater (e.g., by a statistically significant amount, such as about 1.5, 2, 3, 4, 5, 6, 7, 8, 9, 10, 15, 20, 25, 30, 40, 50, 60, 70, 80, 90, or 100-fold or more greater) than the photosynthetic activity of a corresponding wild-type photosynthetic microorganism under the same or comparable stress condition. In particular embodiments, said photosynthetic activity (i.e., for comparison) is measured at about day 1, 2, 3, 4, 5, 6, 7, 8, 9, 10, 11, 12, 13, 14, 15, 16, 17, 18, 19, 20, or 21 post-initiation or re-initiation of the stress condition, or anytime within about days 5-20 post-initiation or re-initiation of the stress condition.

In certain embodiments, maintenance of photosynthetic activity comprises maintenance of CO₂ fixation and/or maintenance of chlorophyll A levels. For instance, in particular embodiments, maintenance of photosynthetic activity includes where chlorophyll A levels of a modified photosynthetic microorganism under stress conditions are at least about 20%, 25%, 30%, 35%, 40%, 45%, 50%, 55%, 60%, 65%, 70%, 75%, 80%, 85%, 90%, 95%, 100%, 120%, 140%, 150%, 160%, 180%, 200% or more of chlorophyll A levels of the modified photosynthetic microorganism under non-stress conditions, such as nitrogen-replete conditions. In some aspects, maintenance of photosynthetic activity includes where chlorophyll A levels of a modified photosynthetic microorganism under stress conditions are substantially greater (e.g., by a statistically significant amount, such as about 1.5, 2, 3, 4, 5, 6, 7, 8, 9, 10, 15, 20, 25, 30, 40, 50, 60, 70, 80, 90, or 100-fold or more greater) than chlorophyll A levels of a corresponding wild-type photosynthetic microorganism under the same or comparable stress condition. In particular embodiments, said chlorophyll A levels (i.e., for comparison) are measured at about day 1, 2, 3, 4, 5, 6, 7, 8, 9, 10, 11, 12, 13, 14, 15, 16, 17, 18, 19, 20, or 21 post-initiation or re-initiation of the stress condition, or anytime within about days 5-20 post-initiation or re-initiation of the stress condition.

In particular embodiments of the various systems and methods of the present invention, the modified photosynthetic organism having reduced glycogen accumulation grows under stress conditions at a rate of less than 60%, less than 50%, less than 40%, less than 30% or less than 20% of its growth rate under non-stress conditions. Accordingly, in particular embodiments, systems and methods of the present invention comprise splitting or reducing the density of cultures less frequently than for a corresponding wild-type or unmodified organism. In particular embodiments, culture are grown for at least 2 days, at least 1 week, at least 2 weeks, at least 1 month, at least 2 months, at least 4 months, at least 6 months, or at least 1 year without being split, diluted or their density reduced by more than 10%.

As described herein, in certain embodiments, systems and methods of the present invention utilize modified photosynthetic organisms that include modifications in addition to those resulting in reduced glycogen, such as modifications to further increase production of a carbon-containing compound described herein. Merely by way of example, certain aspects include modifications to further increase lipid production or secretion (e.g., increase production of fatty acids, fatty alcohols, alkanes), to allow the production of triglycerides and/or wax esters, to facilitate the production of isobutanol or isopentanol, to facilitate the production of 4-hydroxybutyrate and/or 1,4-butanediol, and/or to increase production of polyamine intermediates.

### Modified Photosynthetic Microorganisms

The present invention relates, in part, to the discovery that reducing the expression level of certain genes involved in glycogen synthesis, such as by mutation or deletion, leads to reduced glycogen synthesis and/or storage in modified photosynthetic microorganisms, such as Cyanobacteria, and further uncouples photosynthesis from growth under stress conditions. For instance, Cyanobacteria, such as Synechococcus, which contain deletions of the glucose-1-phosphate adenylyltransferase gene (glgC), the phosphoglucomutase gene (pgm), and/or the glycogen synthase gene (glgA), individually or in various combinations, may produce and accumulate significantly reduced levels of glycogen as compared to wild-type Cyanobacteria. The reduction of glycogen synthesis or accumulation may be especially pronounced under stress conditions, including the reduction of nitrogen. In addition, the present invention further relates to the discovery that the overexpression in photosynthetic microorganisms, including Cyanobacteria, of genes or proteins involved in glycogen breakdown or secretion also leads to reduced glycogen synthesis and/or storage.

Accordingly, the present invention further relates to the discovery that by blocking, disrupting, or down-regulating the natural glycogen synthesis and storage pathway, e.g., by gene mutation or deletion, or by increasing, enhancing, or up-regulating the natural glycogen breakdown pathway in modified photosynthetic organisms, including photosynthetic microorganisms such as Cyanobacteria, the resulting strains of photosynthetic organisms increase carbon flow into other biosynthetic pathways. Examples of other biosynthetic pathways include existing pathways, such as existing lipid biosynthetic pathways, or pathways that are introduced through genetic engineering, such as triglyceride or other carbon-containing compound biosynthesis pathways.

The present invention, therefore, relates generally to modified photosynthetic organisms, including modified Cyanobacteria, and methods of use thereof, which have been modified to produce or store reduced levels of glycogen as compared to wild-type photosynthetic microorganisms. In particular embodiments, the modified photosynthetic organism is genetically modified, for instance, relative to the wild-type or most frequently observed photosynthetic organism of that same species. Genetic modifications can be man-made and/or naturally-occurring, for instance, by direct molecular biological intervention (e.g., cloning or insertion of exogenous genetic elements to reduce expression of genes associated with glycogen synthesis/storage), directed evolution under controlled conditions to enhance natural selection of glycogen-deficient or glycogen-reduced mutants, or identification of spontaneous glycogen-deficient or glycogen-reduced mutants under natural conditions, including combinations thereof.

In certain embodiments, the modified photosynthetic organism has a reduced level of expression of one or more genes of a glycogen biosynthesis or storage pathway and/or overexpresses one or more genes or proteins of a glycogen breakdown pathway, such that said photosynthetic organism synthesizes or accumulates a reduced amount of glycogen, e.g., under stress conditions, e.g., reduced nitrogen, as compared to a wild-type photosynthetic organism. In one embodiment, the modified photosynthetic organism comprises one or more mutations or deletions in one or more genes of a glycogen biosynthesis or storage pathway, which may be, e.g., complete or partial gene deletions. In other embodiments, the modified photosynthetic organism comprises one or more polynucleotides comprising an antisense RNA sequence that targets, e.g., hybridizes to, one or more genes or mRNAs of a glycogen biosynthesis or storage pathway, such as an antisense oligonucleotide or a short interfering RNA (siRNA), or a vector that expresses one or more such polynucleotides.

In particular embodiments, the modified photosynthetic microorganism comprises one or more introduced or overexpressed polynucleotides that encode one or more proteins associated with glycogen breakdown or secretion of glycogen precursors. For instance, modified photosynthetic microorganisms that accumulate reduced glycogen relative to wild-type may comprise one or more introduced or overexpressed polynucleotides that encode one or more of a glycogen phosphorylase (GlgP), glycogen isoamylase (GlgX), glucanotransferase (MalQ), phosphoglucomutase (Pgm), glucokinase (Glk), and/or a phosphoglucose isomerase (Pgi), include functional fragments and variants thereof. Pgm, Glk, and Pgi are bidirectional enzymes that can promote glycogen synthesis or breakdown depending on conditions.

In particular embodiments, the modified photosynthetic organism produces an increased amount of one or more carbon-containing compounds other than glycogen. Exemplary carbon-containing compounds are described herein.

In certain aspects, the modified photosynthetic organisms described herein are further modified to increase production of lipids, for instance, by introducing and/or overexpressing one or more polypeptides associated with lipid biosynthesis. Examples of such lipids include fatty acids, fatty alcohols, fatty aldehydes, alkane/alkenes, triglycerides, and wax esters. Hence, in some instances, modified photosynthetic microorganisms that accumulate a reduced amount of glycogen as compared to the wild-type photosynthetic microorganism can further comprise one or more introduced or overexpressed polynucleotides encoding one or more of an acyl carrier protein (ACP), acyl ACP synthase (Aas), acyl-ACP reductase, alcohol dehydrogenase, aldehyde dehydrogenase, aldehyde decarbonylase, thioesterase (TES), acetyl coenzyme A carboxylase (ACCase), diacylglycerol acyltransferase (DGAT), phosphatidic acid phosphatase (PAP; or phosphatidate phosphatase), triacylglycerol (TAG) hydrolase, fatty acyl-CoA synthetase, lipase/phospholipase, or any combination thereof.

Moreover, by further modifying a given photosynthetic organism of the present invention having a disrupted/reduced glycogen biosynthesis or storage pathway and/or an enhanced glycogen breakdown pathway, so as to increase the production of other carbon-containing compounds, such as lipids, which are necessary for the production of triglycerides, and by also modifying that photosynthetic microorganism to produce triglycerides, certain of the modified photosynthetic microorganism of the present invention can be used to produce higher amounts of triglycerides than would otherwise be possible absent the discovery that disruption of glycogen pathways in photosynthetic microorganism could be utilized to increase the production of other carbon-containing compounds under stress conditions. Certain embodiments thus include modified photosynthetic microorganisms that accumulate a reduced amount of glycogen as compared to the wild-type photosynthetic microorganism, and which comprise one or more introduced polynucleotides that encode an enzyme having DGAT activity. Optionally, to further increase production of triglycerides, such photosyntheticm microorganisms can further comprise one or more introduced or overexpressed polynucleotides that encode a phosphatidate phosphatase, ACCase, ACP, phospholipase B, phospholipase C, fatty acyl Co-A synthetase, or any combination thereof. Specific embodiments include an introduced DGAT in combination with an introduced or overexpressed ACCase, PAP, or both.

Certain embodiments of the present invention relate to modified photosynthetic organisms, including Cyanobacteria, and methods of use thereof, wherein the modified photosynthetic microorganisms further comprise one or more over-expressed, exogenous, or introduced polynucleotides encoding an acyl-ACP reductase polypeptide, or a fragment or variant thereof. In particular embodiments, the fragment or variant thereof retains at least 50% of one or more activities of the wild-type acyl-ACP reductase polypeptide. As with most any of the overexpressed polypeptides described herein, an overexpressed acyl-ACP reductase can be encoded by an endogenous or naturally-occurring polynucleotide which is operably linked to an introduced promoter, typically upstream of the microorganism's natural acyl-ACP reductase coding region, and/or it can be encoded by an introduced polynucleotide that encodes an acyl-ACP reductase.

In certain embodiments, an introduced promoter is inducible, and in some embodiments it is constitutive. Included are weak promoters under non-induced conditions. Exemplary promoters are described elsewhere herein and known in the art. In particular embodiments, the introduced promoter is exogenous or foreign to the photosynthetic microorganism, i.e., it is derived from a genus/species that differs from the microorganism being modified. In other embodiments, the introduced promoter is a recombinantly introduced copy of an otherwise endogenous or naturally-occurring promoter sequence, i.e., it is derived from the same species of microorganism being modified.

Similar principles can apply to the introduced polynucleotide which encodes the acyl-ACP reductase or other overexpressed polypeptide (e.g., aldehyde dehydrogenase). For instance, in particular embodiments, the introduced polynucleotide encoding the acyl-ACP reductase or other polypeptide is exogenous or foreign to the photosynthetic microorganism, i.e., it is derived from a genus/species that differs from the microorganism being modified. In other embodiments, the introduced polynucleotide is a recombinantly introduced copy of an otherwise endogenous or naturally-occurring sequence, i.e., it is derived from the same species of microorganism being modified.

Acyl-ACP reductase polypeptides, and fragments and variants thereof, that may be used according to the compositions and methods of the present invention are described herein. The present invention contemplates the use of naturally-occurring and non-naturally-occurring variants of these acyl-ACP reductase and other lipid biosynthesis proteins (e.g., ACP, ACCase, DGAT, acyl-CoA synthetase, aldehyde dehydrogenase), as well as variants of their encoding polynucleotides. These enzyme encoding sequences may be derived from any microorganism (e.g., plants, bacteria) having a suitable sequence, and may also include any man-made variants thereof, such as any optimized coding sequences (i.e., codon-optimized polynucleotides) or optimized polypeptide sequences.

Acyl-ACP reductase polypeptides may also be overexpressed in strains of photosynthetic microorganisms that have been modified to overexpress one or more selected lipid biosynthesis proteins (e.g., selected fatty acid biosynthesis proteins, triacylglycerol biosynthesis proteins, alkane/alkene biosynthesis proteins, wax ester biosynthesis proteins).

For example, to produce triglycerides, a modified photosynthetic microorganism may comprise an overexpressed acyl-ACP reductase in combination with an introduced polynucleotide that encodes a DGAT. In these and related embodiments, triglyceride production can be further increased by introduction or overexpression of an aldehyde dehydrogenase, for instance, to increase production of fatty acids, the precursors to triglycerides. One exemplary aldehyde dehydrogenase is encoded by orf0489 of Synechococcus elongatus PCC7942. Also included are homologs or paralogs thereof, functional equivalents thereof, and fragments or variants thereofs. Functional equivalents can include aldehyde dehydrogenases with the ability to convert acyl aldehydes (e.g., nonyl-aldehyde) into fatty acids. In specific embodiments, the aldehyde dehydrogenase has the amino acid sequence of SEQ ID NO:103 (encoded by the polynucleotide sequence of SEQ ID NO:102), or an active fragment or variant of this sequence. These and related embodiments can be further combined with reduced expression and/or activity of an endogenous aldehyde decarbonylase (e.g., orf1593 in S. elongatus), described herein, to shunt carbon away from alkanes and towards fatty acids, the precursors to triglycerides.

To produce wax esters, a modified photosynthetic microorganism may comprise an overexpressed acyl-ACP reductase and an introduced polynucleotide that encodes a DGAT (e.g., a bi-functional DGAT having wax ester synthase activity) in further combination with an introduced or overexpressed polynucleotide that encodes an alcohol dehydrogenase, such as a long-chain alcohol dehydrogenase. Exemplary alcohol dehydrogenases include slr1192 from Synechycystis sp. PC06083 and ACIAD3612 from Acinetobacter baylyi (see SEQ ID NOS:104-107). Also included are homologs or paralogs thereof, functional equivalents thereof, and fragments or variants thereofs. Functional equivalents can include alcohol dehydrogenases with the ability to convert acyl aldehydes (e.g., nonyl-aldehyde, O₁₂, O₁₄, O₁₆, O₁₈, O₂₀ fatty aldehydes) into fatty alcohols, which can then be converted into wax esters by the wax ester synthase. In specific embodiments, the alcohol dehydrogenase has the amino acid sequence of SEQ ID NO:105 (slr1192; encoded by the polynucleotide sequence of SEQ ID NO:104), or an active fragment or variant of this sequence. In some embodiments, the alcohol dehydrogenase has the amino acid sequence of SEQ ID NO:107 (ACIAD3612; encoded by the polynucleotide sequence of SEQ ID NO:106), or an active fragment or variant of this sequence. Certain of these and related embodiment can be combined with any one or more of reduced expression and/or activity of an endogenous aldehyde dehydrogenase (e.g., orf0489 deletion) to shunt carbon away from fatty acid production, reduced expression and/or activity of an endogenous aldehyde decarbonylase (e.g., orf1593 deletion) to shunt carbon away from alkane production, or both. Also included are combinations that further comprise an introduced or overexpressed acyl carrier protein (ACP), optionally in combination with an introduced or overexpressed acyl-ACP synthetase (Aas).

To produce fatty alcohols, a modified photosynthetic microorganism may comprise an overexpressed acyl-ACP reductase in combination with an introduced or overexpressed alcohol dehydrogenase. These and related embodiments can be further combined with reduced expression and/or activity of an endogenous aldehyde decarbonylase (e.g., orf1593 from S. elongatus), reduced expression and/or activity of an endogenous aldehyde dehydrogenase (e.g., orf0489 from S. elongatus), or both, to respectively shunt carbon away from alkanes/alkenes and fatty acids and towards fatty alcohols.

To produce alkanes and/or alkenes, a modified photosynthetic microorganism may comprise an overexpressed acyl-ACP reductase in combination with an introduced or overexpressed aldehyde decarbonylase. Exemplary aldehyde decarbonylases include that encoded by orf1593 of S. elongatus PCC7942 and its orthologs/paralogs, including those found in Synechocystis sp. PCC6803 (encoded by orfsll0208), N. punctiforme PCC 73102, Thermosynechococcus elongatus BP-1, Synechococcus sp. Ja-3-3AB, P. marinus MIT9313, P. marinus NATL2A, and Synechococcus sp. RS 9117, the latter having at least two paralogs (RS 9117-1 and -2). These and related embodiments can be further combined with reduced expression and/or activity of an endogenous aldehyde dehydrogenase (e.g., orf0489 from S. elongatus), reduced expression and/or activity of an endogenous alcohol dehydrogenase (e.g., a long-chain alcohol dehydrogenase), or both, to respectively shunt carbon away from fatty acids and fatty alcohols and towards alkanes and/or alkenes.

To produce fatty acids, such as free fatty acids, a modified photosynthetic microorganism may comprise an overexpressed acyl-ACP reductase in optional combination with an introduced or overexpressed aldehyde dehydrogenase (e.g., orf 0489 from S. elongatus or orthologs/paralogs/homologs thereof). These and related embodiments can be further combined with reduced expression and/or activity of an aldehyde decarbonylase (e.g., orf1593 from S. elongatus), reduced expression and/or activity of an endogenous alcohol dehydrogenase (e.g., long-chain alcohol dehydrogenase), or both, to respectively shunt carbon away from alkanes and fatty alcohols and towards fatty acids. In certain embodiments, such as Cyanobacteria including S. elongatus PCC7942, orf1593 resides directly upstream of orf1594 (acyl-ACP reductase coding region) and encodes an aldehyde decarbonylase. According to one non-limiting theory, because the aldehyde decarbonylase encoded by orf1593 utilizes acyl aldehyde as a substrate for alkane production, reducing expression of this protein may further increase yields of free fatty acids by shunting acyl aldehydes (e.g., produced by acyl-ACP reductase) away from an alkane-producing pathway, and towards a fatty acid- or fatty alcohol-producing and storage pathway. PCC7942_orf1593 orthologs can be found, for example, in Synechocystis sp. PCC6803 (encoded by orfsll0208), N. punctiforme PCC 73102, Thermosynechococcus elongatus BP-1, Synechococcus sp. Ja-3-3AB, P. marinus MIT9313, P. marinus NATL2A, and Synechococcus sp. RS 9117, the latter having at least two paralogs (RS 9117-1 and -2). Included are strains having mutations or full or partial deletions of one or more genes encoding these and other aldehyde decarbonylases, such as S. elongatus PCC7942 having a full or partial deletion of orf1593, and Synechocystis sp. PCC6803 having a full or partial deletion of orfsll0208). For instance, an exemplary modified photosynthetic microorganism could comprise an overexpressed acyl-ACP reductase, combined with a full or partial deletion of the glgC gene, the glgA gene, and/or the pgm gene, optionally combined with an overexpressed aldehyde dehydrogenase, and optionally combined with a full or partial deletion of a gene encoding an aldehyde decarbonylase (e.g., PCC7942_orf1593, PCC6803_orfsll0208).

Other combinations include, for example, a modified photosynthetic microorganism comprising reduced glycogen accumulation, in combination with one more of an overexpressed ACP; an overexpressed acyl-ACP reductase in combination with an overexpressed ACP; an acyl-ACP reductase on combination with an ACCase; an acyl-ACP reductase on combination with an ACP and an ACCase; an overexpressed acyl-ACP reductase in combination with an overexpressed DGAT and optionally an overexpressed acyl-CoA synthetase (e.g., a DGAT/acyl-CoA synthetase combination); an overexpressed acyl-ACP reductase with an overexpressed ACP and an overexpressed DGAT, optionally combined with an overexpressed acyl-CoA synthetase; an overexpressed acyl-ACP reductase with an overexpressed ACCase and an overexpressed DGAT, optionally in combination with an overexpressed acyl-CoA synthetase; and an overexpressed acyl-ACP reductase with an overexpressed ACP, ACCase, and an overexpressed DGAT, optionally in combination with an overexpressed acyl-CoA synthetase. Acyl-ACP reductase and DGAT-overexpressing strains, optionally in combination with an overexpressed acyl-CoA synthetase, typically produce increased triglycerides relative to DGAT-only overexpressing strains.

Any one of these embodiments can also be combined with a strain having reduced expression of an acyl-ACP synthetase (Aas). Without wishing to be bound by any one theory, an endogenous aldehyde dehydrogenase is acting on the acyl-aldehydes generated by orf1594 and converting them to free fatty acids. The normal role of such a dehydrogenase might involve removing or otherwise dealing with damaged lipids. In this scenario, it is then likely that the Aas gene product recycles these free fatty acids by ligating them to ACP. Accordingly, reducing or eliminating expression of the Aas gene product might ultimately increase production of fatty acids and thus optionally triglycerides (e.g., in a DGAT-expressing microorganism), by reducing or preventing their transfer to ACP. Included are mutations and full or partial deletions of one or more Aas genes, such as the Aas gene of Synechococcus elongatus PCC 7942. As one example, a specific modified photosynthetic microorganism could comprise an overexpressed acyl-ACP reductase, combined with a full or partial deletion of the glgC gene, the glgA gene, and/or the pgm gene, optionally combined with an overexpressed ACP, ACCase, DGAT/acyl-CoA synthetase, or all of the foregoing, optionally combined with a full or partial deletion of a gene encoding an aldehyde decarbonylase (e.g., PCC7942_orf1593, PCC6803_orfsll0208), and optionally combined with a full or partial deletion of an Aas gene encoding an acyl-ACP synthetase.

Certain embodiments of the systems and methods of the present invention utilize modified photosynthetic organisms with reduced glycogen accumulation that are further modified to allow production of isobutanol or isopentanol. In particular embodiments, these organisms comprise one or more introduced or overexpressed polynucleotides that encode a polypeptide associated with isobutanol or isopentanol production. Examples of such polynucleotides include the genes required to convert a 2-keto acid to an aldehyde (2-keto acid decarboxylase) and then convert the aldehyde to an alcohol (alcohol dehydrogenase) in Synechococcus elongatus, according to Atsumi and Liao 2007 Nature and 2009 Nature Biotech. Expression of these genes, or functional fragments or variants thereof, should allow for the production of isobutanol or isopentanol (3-methyl-1-butanol). In specific embodiments, these genes are Alpha-ketoisovalerate decarboxylase (2-keto acid decarboxylase) from Lactococcus lactis (kivd) and Alcohol dehydrogenase from E. coli (YqhD). The polynucleotide sequence of Alpha-ketoisovalerate decarboxylase (2-keto acid decarboxylase) from Lactococcus lactis is set forth in SEQ ID NO:180, and its encoded polypeptide sequence is set forth in SEQ ID NO:181. The polynucleotide sequence of alcohol dehydrogenase from E. coli (YqhD) is set forth in SEQ ID NO:182, and its encoded polypeptide sequence is set forth in SEQ ID NO:183.

In additional related embodiments, the modified photosynthetic organism with reduced glycogen accumulation are further modified to include one or more introduced or overexpressed polynucleotides involved in converting pyruvate to the precursors for isobutanol or isopentanol production. Thus, they may also be used in combination with any of the related modifications described above. Examples of such polynucleotides and encoded polypeptides include, acetolactate synthase (e.g., Synechococcus elongatus PCC7942 ilvN (NCBI YP_{—}401451; SEQ ID NO:184)), acetolactate synthase (e.g., Synechococcus elongatus PCC7942 ilvB (NCBI YP_{—}399158; SEQ ID NO:185)), ketol-acid reductoisomerase (e.g., Synechococcus elongatus PCC7942 ilvC (NCBI YP_{—}400569; SEQ ID NO:186), dihydroxy-acid dehydratase (e.g., Synechococcus elongatus PCC7942 ilvD (NCBI YP_{—}399645; SEQ ID NO:187)), 2-isopropylmalate synthase (e.g., Synechococcus elongatus PCC7942 leuA1 (NCBI YP_{—}399447; SEQ ID NO: 188)); 2-isopropylmalate synthase (e.g., Synechococcus elongatus PCC7942 leuA2 (NCBI YP_{—}400427; SEQ ID NO: 189)), isopropylmalate dehydratase (e.g., Synechococcus elongatus PCC7942 leuD (NCBIYP_{—}401565; SEQ ID NO:190)), isopropylmalate dehydratase (e.g., Synechococcus elongatus PCC7942 leuC (NCBI YP_{—}400915; SEQ ID NO:191)), 3-isopropylmalate dehydrogenase (e.g., Synechococcus elongatus PCC7942 leuB (NCBI YP_{—}400522; SEQ ID NO:192); acetolactate synthase (e.g., Bacillus subtilus 168 alsS (NCBI NP_{—}391482; SEQ ID NO:193)); ketol-acid reductoisomerase, NAD(P)-binding (e.g., E. coli K-12, MG1655 ilvC (NCBI NP_{—}418222; SEQ ID NO:194)); and dihydroxyacid dehydratase (e.g., E. coli K-12, MG1655 ilvD (NCBI_YP_{—}026248; SEQ ID NO:195)) and functional fragments and variants thereof.

In additional embodiments, the modified photosynthetic organism with reduced glycogen accumulation are further modified to include one or more introduced or overexpressed polynucleotides involved in glucose secretion, in order to allow for continued secretion of glucose from glycogen deficient strains that are placed under stress conditions. Examples of such polynucleotides and encoded polypeptides are glucose permeases and glucose/H+ symporters, such as glcP (e.g., Bacillus subtilis168 glcP; NCBI NP_{—}388933; SEQ ID NO:176), glcP1 (e.g., Streptomyces coelicolor glcP1; NCBI NP_{—}629713.1; SEQ ID NO:177), glcP2 (e.g., Streptomyces coelicolor A3 glcP2; NCBI NP_{—}631212; SEQ ID NO:178), and Mycobacterium smegmatis MC2 155 (NCBI YP_{—}888461; SEQ ID NO:179), and functional fragments and variants thereof.

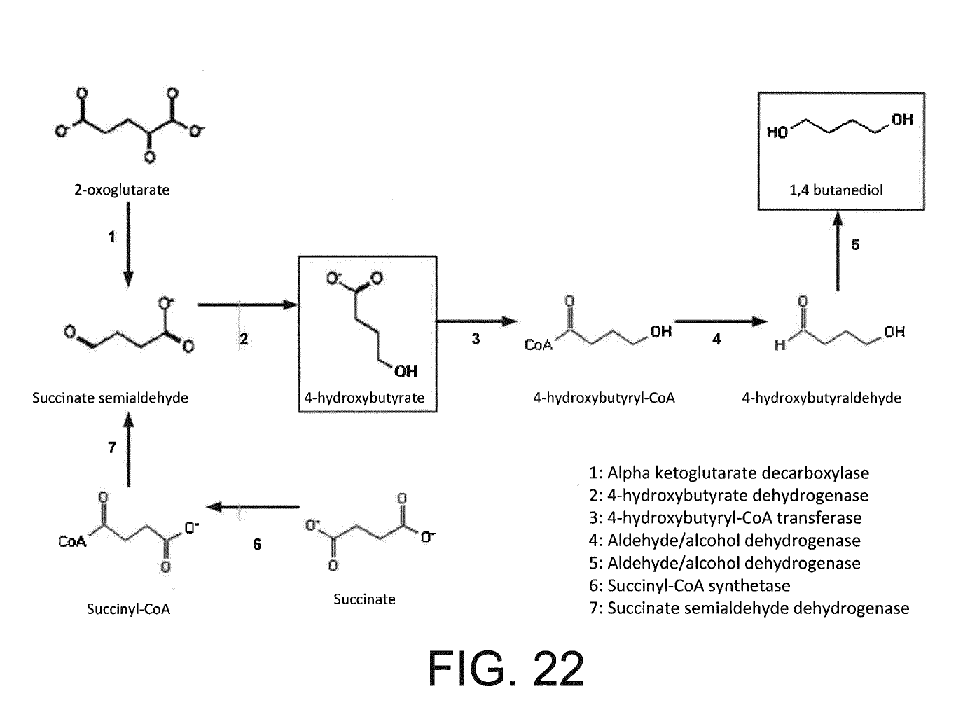

Certain embodiments of the systems and methods of the present invention utilize modified photosynthetic organisms with reduced glycogen accumulation that are further modified to allow production of 4-hydroxybutyrate. In particular embodiments, these photosynthetic organisms comprise one or more introduced or overexpressed polynucleotides that encode a polypeptide associated with 4-hydroxybutyrate production. Examples of such polynucleotides include the genes required to convert 2-oxogluturate into succinate semialdehyde, and then convert the latter into 4-hydroxybutyrate. In particular embodiments, an alpha-ketoglutarate decarboxylase converts 2-oxogluturate into succinate semialdehyde and a 4-hydroxybutyrate dehydrogenase converts succinate semialdehyde into 4-hydroxybutyrate. Additional examples of such polynucleotides include the genes required to convert succinate into succinyl-CoA, convert succinyl-CoA into succinate semialdehyde, and then conver the latter into 4-hydroxybutyrate. In particular embodiments, a succinyl-CoA synthetase converts succinate into succinyl-CoA, a succinate-semialdehyde dehydrogenase converts succinyl-CoA into succinate semialdehyde, and a 4-hydroxybutyrate dehydrogenase converts succinate semialdehyde into 4-hydroxybutyrate. Specific examples of alpha-ketoglutarate decarboxylases include those encoded by CCDC5180_{—}0513 (SEQ ID NO:199) from Mycobacterium bovis and SYNPCC7002_A2770 (SEQ ID NO:201) from Synechococcus sp PCC 7002. Specific examples of 4-hydroxybutyrate dehydrogenases include those encoded by PGN_{—}0724 (SEQ ID NO:203) from Porphyromonas gingivalis and CKR_{—}2662 (SEQ ID NO:205) from Clostridium kluyveri. Specific examples of succinyl-CoA synthetases include the succinyl-CoA synthetase-alpha subunit encoded by sucC (b0728) (SEQ ID NO:213) from E. coli and the succinyl-CoA synthetase-beta subunit encoded by sucD (b0729) (SEQ ID NO:215) from E. coli. Specific examples of succinate-semialdehyde dehydrogenases include that encoded by PGTDC60_{—}1813 (SEQ ID NO:217) from Porphyromonas gingivalis. Expression of certain combinations of these or related genes, or functional fragments or variants thereof, should allow for the production of 4-hydroxybutyrate from 2-oxogluturate or succinate (see FIG. 22).

Certain embodiments of the systems and methods of the present invention utilize modified photosynthetic organisms with reduced glycogen accumulation that are further modified to allow production of 4-hydroxybutyrate and optionally 1,4-butanediol. In some embodiments, and further to the polypeptides associated with the production of 4-hydroxybutyrate (supra), these microorganisms comprise one or more introduced or overexpressed polynucleotides that encode a polypeptide associated with the production of 1,4-butanediol from 4-hydroxybutyrate. Examples of such polynucleotides include the genes required to convert 4-hydroxybutyrate into 4-hydroxybutyryl-CoA, then convert 4-hydroxybutyryl-CoA into 4-hydroxybutyraldehyde, and then convert 4-hydroxybutyraldehyde into 1,4-butanediol. In particular embodiments, a 4-hydroxybutyryl-CoA transferase converts 4-hydroxybutyrate into 4-hydroxybutyryl-CoA, an aldehyde/alcohol dehydrogenase converts 4-hydroxybutyryl-CoA into 4-hydroxybutyraldehyde (e.g., one that is capable of reducing coA-linked substrates to aldehydes/alcohols), and an aldehyde/alcohol dehydrogenase converts 4-hydroxybutyraldehyde into 1,4-butanediol. Specific examples of 4-hydroxybutyryl-CoA transferases include that encoded by cat2 (CKR_{—}2666) (SEQ ID NO:207) from Clostridium kluyveri, including homologs from Clostridium aminobutyricum and Porphyromonas gingivalis. Specific examples of aldehyde/alcohol dehydrogenases include those encoded by adhE2 (CEA_P0034) (SEQ ID NO:209) from Clostridium acetobutylicum and adhE (b1241) (SEQ ID NO:211) from E. coli. Expression of certain combinations of these or related genes, or functional fragments or variants thereof, should allow for the production of 4-hydroxybutyrate from 2-oxogluturate or succinate, and the production of 1,4-butanediol from 4-hydroxybutyrate (see FIG. 22).

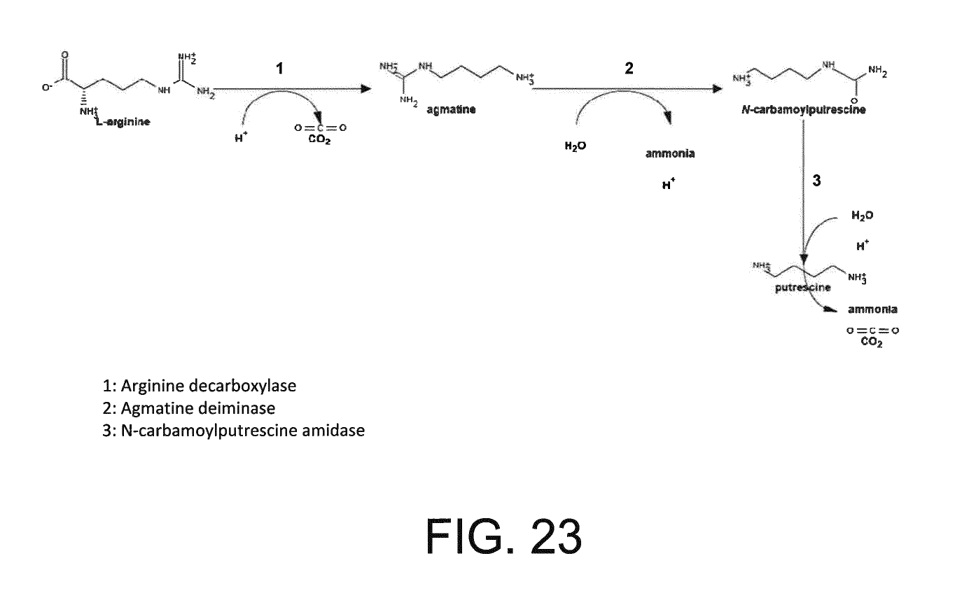

Particular embodiments of the systems and methods of the present invention utilize modified photosynthetic organisms with reduced glycogen accumulation that are further modified to allow production of polyamine intermediates/precursors. Exemplary polyamine intermediates include agmatine and putrescine. As shown in the accompanying Examples, the systems and methods described herein can produce increased agmatine and putrescine without any further modifications. However, in particular embodiments, to further increase production these microorganisms may comprise one or more introduced or overexpressed polynucleotides that encode a polypeptide associated with polyamine intermediate production. Examples of such polynucleotides include the genes required to convert L-arginine into agmatine, and optionally the genes required to convert agmatine into N-carbamoylputrescine, and then convert N-carbamoylputrescine into putrescine. In some embodiments, an arginine decarboxylase is introduced or overexpressed to convert L-arginine into agmatine. In particular embodiments, an agmatine deiminase is introduced or overexpressed to convert agmatine into N-carbamoylputrescine, and/or a N-carbamoylputrescine amidase is introduced or overexpressed to convert N-carbamoylputrescine into putrescine. Specific examples of arginine decarboxylases include that encoded by Synpcc7942_{—}1037 (SEQ ID NO:219) from S. elongatus PCC7942. Specific examples of agmatine deiminases include that encoded by Synpcc7942_{—}2402 (SEQ ID NO:221) and Synpcc7942_{—}2461 from S. elongatus PCC7942. Specific examples of N-carbamoylputrescine amidases include that encoded by Synpcc7942_{—}2145 (SEQ ID NO:223) from S. elongatus PCC7942. Introduction or overexpression of certain combinations of these or related genes, or functional fragments or variants thereof, should allow for the increased production of agmatine, putrescine, or both (see FIG. 23).

Increased expression can be achieved a variety of ways, for example, by introducing a polynucleotide into the photosynthetic organism, modifying an endogenous gene to overexpress the polypeptide, or both. For instance, one or more copies of an otherwise endogenous polynucleotide sequence can be introduced by recombinant techniques to increase expression, and/or a promoter/enhancer sequence can be introduced upstream of an endogenous gene to regulate expression.

Modified photosynthetic organisms of the present invention may be produced, for example, using any type of photosynthetic microorganism. These include, but are not limited to photosynthetic bacteria, green algae, and Cyanobacteria. The photosynthetic microorganism can be, for example, a naturally photosynthetic microorganism, such as a Cyanobacterium, or an engineered photosynthetic microorganism, such as an artificially photosynthetic bacterium.

Exemplary microorganisms that are either naturally photosynthetic or can be engineered to be photosynthetic include, but are not limited to, bacteria; fungi; archaea; protists; eukaryotes, such as a green algae; and animals such as plankton, planarian, and amoeba. Examples of naturally occurring photosynthetic microorganisms include, but are not limited to, Spirulina maximum, Spirulina platensis, Dunaliella salina, Botrycoccus braunii, Chlorella vulgaris, Chlorella pyrenoidosa, Serenastrum capricomutum, Scenedesmus auadricauda, Porphyridium cruentum, Scenedesmus acutus, Dunaliella sp., Scenedesmus obliquus, Anabaenopsis, Aulosira, Cylindrospermum, Synechococcus sp., Synechocystis sp., and/or Tolypothrix.

A modified Cyanobacteria of the present invention may be from any genera or species of Cyanobacteria that is genetically manipulable, i.e., permissible to the introduction and expression of exogenous genetic material. Examples of Cyanobacteria that can be engineered according to the methods of the present invention include, but are not limited to, the genus Synechocystis, Synechococcus, Thermosynechococcus, Nostoc, Prochlorococcu, Microcystis, Anabaena, Spirulina, and Gloeobacter.

Cyanobacteria, also known as blue-green algae, blue-green bacteria, or Cyanophyta, is a phylum of bacteria that obtain their energy through photosynthesis. Cyanobacteria can produce metabolites, such as carbohydrates, proteins, lipids and nucleic acids, from CO₂, water, inorganic salts and light. Any Cyanobacteria may be used according to the present invention.

Cyanobacteria include both unicellular and colonial species. Colonies may form filaments, sheets or even hollow balls. Some filamentous colonies show the ability to differentiate into several different cell types, such as vegetative cells, the normal, photosynthetic cells that are formed under favorable growing conditions; akinetes, the climate-resistant spores that may form when environmental conditions become harsh; and thick-walled heterocysts, which contain the enzyme nitrogenase, vital for nitrogen fixation.

Heterocysts may also form under the appropriate environmental conditions (e.g., anoxic) whenever nitrogen is necessary. Heterocyst-forming species are specialized for nitrogen fixation and are able to fix nitrogen gas, which cannot be used by plants, into ammonia (NH₃), nitrites (NO₂⁻), or nitrates (NO₃⁻), which can be absorbed by plants and converted to protein and nucleic acids.

Many Cyanobacteria also form motile filaments, called hormogonia, which travel away from the main biomass to bud and form new colonies elsewhere. The cells in a hormogonium are often thinner than in the vegetative state, and the cells on either end of the motile chain may be tapered. In order to break away from the parent colony, a hormogonium often must tear apart a weaker cell in a filament, called a necridium.

Each individual Cyanobacterial cell typically has a thick, gelatinous cell wall. Cyanobacteria differ from other gram-negative bacteria in that the quorum sensing molecules autoinducer-2 and acyl-homoserine lactones are absent. They lack flagella, but hormogonia and some unicellular species may move about by gliding along surfaces. In water columns, some Cyanobacteria float by forming gas vesicles, like in archaea.

Cyanobacteria have an elaborate and highly organized system of internal membranes that function in photosynthesis. Photosynthesis in Cyanobacteria generally uses water as an electron donor and produces oxygen as a by-product, though some Cyanobacteria may also use hydrogen sulfide, similar to other photosynthetic bacteria. Carbon dioxide is reduced to form carbohydrates via the Calvin cycle. In most forms, the photosynthetic machinery is embedded into folds of the cell membrane, called thylakoids. Due to their ability to fix nitrogen in aerobic conditions, Cyanobacteria are often found as symbionts with a number of other groups of microorganisms such as fungi (e.g., lichens), corals, pteridophytes (e.g., Azolla), and angiosperms (e.g., Gunnera), among others.

Cyanobacteria are the only group of microorganisms that are able to reduce nitrogen and carbon in aerobic conditions. The water-oxidizing photosynthesis is accomplished by coupling the activity of photosystem (PS) II and I (Z-scheme). In anaerobic conditions, Cyanobacteria are also able to use only PS I (i.e., cyclic photophosphorylation) with electron donors other than water (e.g., hydrogen sulfide, thiosulphate, or molecular hydrogen), similar to purple photosynthetic bacteria. Furthermore, Cyanobacteria share an archaeal property; the ability to reduce elemental sulfur by anaerobic respiration in the dark. The Cyanobacterial photosynthetic electron transport system shares the same compartment as the components of respiratory electron transport. Typically, the plasma membrane contains only components of the respiratory chain, while the thylakoid membrane hosts both respiratory and photosynthetic electron transport.

Phycobilisomes, attached to the thylakoid membrane, act as light harvesting antennae for the photosystems of Cyanobacteria. The phycobilisome components (phycobiliproteins) are responsible for the blue-green pigmentation of most Cyanobacteria. Color variations are mainly due to carotenoids and phycoerythrins, which may provide the cells with a red-brownish coloration. In some Cyanobacteria, the color of light influences the composition of phycobilisomes. In green light, the cells accumulate more phycoerythrin, whereas in red light they produce more phycocyanin. Thus, the bacteria appear green in red light and red in green light. This process is known as complementary chromatic adaptation and represents a way for the cells to maximize the use of available light for photosynthesis.

In particular embodiments, the Cyanobacteria may be, e.g., a marine form of Cyanobacteria or a fresh water form of Cyanobacteria. Examples of marine forms of Cyanobacteria include, but are not limited to Synechococcus WH8102, Synechococcus RCC307, Synechococcus NKBG 15041c, and Trichodesmium. Examples of fresh water forms of Cyanobacteria include, but are not limited to S. elongatus PCC7942, Synechocystis PCC6803, Plectonema boryanum, and Anabaena sp. Exogenous genetic material encoding the desired enzymes or polypeptides may be introduced either transiently, such as in certain self-replicating vectors, or stably, such as by integration (e.g., recombination) into the Cyanobacterium's native genome.

In other embodiments, a genetically modified Cyanobacteria of the present invention may be capable of growing in brackish or salt water. When using a fresh water form of Cyanobacteria, the overall net cost for production of triglycerides will depend on both the nutrients required to grow the culture and the price for freshwater. One can foresee freshwater being a limited resource in the future, and in that case it would be more cost effective to find an alternative to freshwater. Two such alternatives include: (1) the use of waste water from treatment plants; and (2) the use of salt or brackish water.

Salt water in the oceans can range in salinity between 3.1% and 3.8%, the average being 3.5%, and this is mostly, but not entirely, made up of sodium chloride (NaCl) ions. Brackish water, on the other hand, has more salinity than freshwater, but not as much as seawater. Brackish water contains between 0.5% and 3% salinity, and thus includes a large range of salinity regimes and is therefore not precisely defined. Waste water is any water that has undergone human influence. It consists of liquid waste released from domestic and commercial properties, industry, and/or agriculture and can encompass a wide range of possible contaminants at varying concentrations.

There is a broad distribution of Cyanobacteria in the oceans, with Synechococcus filling just one niche. Specifically, Synechococcus sp. PCC 7002 (formerly known as Agmenellum quadruplicatum strain PR-6) grows in brackish water, is unicellular and has an optimal growing temperature of 38° C. While this strain is well suited to grow in conditions of high salt, it will grow slowly in freshwater. In particular embodiments, the present invention contemplates the use of a Cyanobacteria S. elongatus PCC7942, altered in a way that allows for growth in either waste water or salt/brackish water. S. elongatus PCC7942 mutant resistant to sodium chloride stress has been described (Bagchi, S. N. et al., Photosynth Res. 2007, 92:87-101), and a genetically modified S. elongatus PCC7942 tolerant of growth in salt water has been described (Waditee, R. et al., PNAS 2002, 99:4109-4114). According to the present invention, a salt water tolerant strain is capable of growing in water or media having a salinity in the range of 0.5% to 4.0% salinity, although it is not necessarily capable of growing in all salinities encompassed by this range. In one embodiment, a salt tolerant strain is capable of growth in water or media having a salinity in the range of 1.0% to 2.0% salinity. In another embodiment, a salt water tolerant strain is capable of growth in water or media having a salinity in the range of 2.0% to 3.0% salinity.

Examples of Cyanobacteria that may be utilized and/or genetically modified according to the methods described herein include, but are not limited to, Chroococcales Cyanobacteria from the genera Aphanocapsa, Aphanothece, Chamaesiphon, Chroococcus, Chroogloeocystis, Coelosphaerium, Crocosphaera, Cyanobacterium, Cyanobium, Cyanodictyon, Cyanosarcina, Cyanothece, Dactylococcopsis, Gloecapsa, Gloeothece, Merismopedia, Microcystis, Radiocystis, Rhabdoderma, Snowella, Synychococcus, Synechocystis, Thermosenechococcus, and Woronichinia; Nostacales Cyanobacteria from the genera Anabaena, Anabaenopsis, Aphanizomenon, Aulosira, Calothrix, Coleodesmium, Cyanospira, Cylindrospermosis, Cylindrospermum, Fremyella, Gleotrichia, Microchaete, Nodularia, Nostoc, Rexia, Richelia, Scytonema, Sprirestis, and Toypothrix; Oscillatoriales Cyanobacteria from the genera Arthrospira, Geitlerinema, Halomicronema, Halospirulina, Katagnymene, Leptolyngbya, Limnothrix, Lyngbya, Microcoleus, Oscillatoria, Phormidium, Planktothricoides, Planktothrix, Plectonema, Pseudoanabaena/Limnothrix, Schizothrix, Spirulina, Symploca, Trichodesmium, Tychonema; Pleurocapsales cyanobacterium from the genera Chroococcidiopsis, Dermocarpa, Dermocarpella, Myxosarcina, Pleurocapsa, Stanieria, Xenococcus; Prochlorophytes Cyanobacterium from the genera Prochloron, Prochlorococcus, Prochlorothrix; and Stigonematales cyanobacterium from the genera Capsosira, Chlorogeoepsis, Fischerella, Hapalosiphon, Mastigocladopsis, Nostochopsis, Stigonema, Symphyonema, Symphonemopsis, Umezakia, and Westiellopsis. In certain embodiments, the Cyanobacterium is from the genus Synechococcus, including, but not limited to Synechococcus bigranulatus, Synechococcus elongatus, Synechococcus leopoliensis, Synechococcus lividus, Synechococcus nidulans, and Synechococcus rubescens.

In certain embodiments, the Cyanobacterium is Anabaena sp. strain PCC 7120, Synechocystis sp. strain PCC6803, Nostoc muscorum, Nostoc ellipsosporum, or Nostoc sp. strain PCC 7120. In certain preferred embodiments, the Cyanobacterium is S. elongatus sp. strain PCC7942.

Additional examples of Cyanobacteria that may be utilized in the methods provided herein include, but are not limited to, Synechococcus sp. strains WH7803, WH8102, WH8103 (typically genetically modified by conjugation), Baeocyte-forming Chroococcidiopsis spp. (typically modified by conjugation/electroporation), non-heterocyst-forming filamentous strains Planktothrix sp., Plectonema boryanum M101 (typically modified by electroporation), and Heterocyst-forming strains Anabaena sp. strains ATCC 29413 (typically modified by conjugation), Tolypothrix sp. strain PCC 7601 (typically modified by conjugation/electroporation) and Nostoc punctiforme strain ATCC 29133 (typically modified by conjugation/electroporation).

In certain preferred embodiments, the Cyanobacterium may be S. elongatus sp. strain PCC7942 or Synechococcus sp. PCC 7002 (originally known as Agmenellum quadruplicatum).

In particular embodiments, the genetically modified, photosynthetic microorganism, e.g., Cyanobacteria, of the present invention may be used to produce triglycerides and/or other carbon-containing compounds from just sunlight, water, air, and minimal nutrients, using routine culture techniques of any reasonably desired scale. In particular embodiments, the present invention contemplates using spontaneous mutants of photosynthetic microorganisms that demonstrate a growth advantage under a defined growth condition. Among other benefits, the ability to produce large amounts of triglycerides from minimal energy and nutrient input makes the modified photosynthetic microorganism, e.g., Cyanobacteria, of the present invention a readily manageable and efficient source of feedstock in the subsequent production of biofuels, such as biodiesel, and other specialty chemicals, such as glycerin.

### Methods of Producing Modified Photosynthetic Microorganisms

Methods of producing a modified photosynthetic microorganism, e.g., a Cyanobacterium, that accumulates a reduced amount of glycogen under stress conditions, e.g., reduced nitrogen, as compared to a wild-type photosynthetic microorganism, which may be used in the systems or methods of the present invention, include modifying the photosynthetic microorganism so that it has a reduced level of expression of one or more genes of a glycogen biosynthesis or storage pathway. In certain embodiments, said one or more genes include glucose-1-phosphate adenyltransferase (glgC), phosphoglucomutase (pgm), and/or glycogen synthase (glgA). In particular embodiments, expression or activity is reduced by mutating or deleting a portion or all of said one or more genes. In particular embodiments, expression or activity is reduced by knocking out or knocking down one or more alleles of said one or more genes. In particular embodiments, expression or activity of the one or more genes is reduced by contacting the photosynthetic microorganism with an antisense oligonucleotide or interfering RNA, e.g., an siRNA, that targets said one or more genes. In particular embodiments, a vector that expresses a polynucleotide that hybridizes to said one or more genes, e.g., an antisense oligonucleotide or an siRNA is introduced into said photosynthetic microorganism.

Photosynthetic microorganisms, e.g., Cyanobacteria may be genetically modified according to techniques known in the art, e.g., to delete a portion or all of a gene or to introduce a polynucleotide that expresses a functional polypeptide. As noted above, in certain aspects, genetic manipulation in photosynthetic microorganisms, e.g., Cyanobacteria, can be performed by the introduction of non-replicating vectors which contain native photosynthetic microorganism sequences, exogenous genes of interest, and selectable markers or drug resistance genes. Upon introduction into the photosynthetic microorganism, the vectors may be integrated into the photosynthetic microorganism's genome through homologous recombination. In this way, an exogenous gene of interest and the drug resistance gene are stably integrated into the photosynthetic microorganism's genome. Such recombinants cells can then be isolated from non-recombinant cells by drug selection. Cell transformation methods and selectable markers for Cyanobacteria are also well known in the art (see, e.g., Wirth, Mol Gen Genet 216:175-7, 1989; and Koksharova, Appl Microbiol Biotechnol 58:123-37, 2002; and THE CYANOBACTERIA: MOLECULAR BIOLOGY, GENETICS, AND EVOLUTION (eds. Antonio Herrera and Enrique Flores) Caister Academic Press, 2008, each of which is incorporated by reference for their description on gene transfer into Cyanobacteria, and other information on Cyanobacteria).

Generation of deletions or mutations of any of the one or more genes associated with the glycogen biosynthesis or storage or lipid biosynthesis can be accomplished according to a variety of methods known in the art, including those described and exemplified herein. For instance, the instant application describes the use of a non-replicating, selectable vector system that is targeted to the upstream and downstream flanking regions of a given gene (e.g., glgC, pgm), and which recombines with the Cyanobacterial genome at those flanking regions to replace the endogenous coding sequence with the vector sequence. Given the presence of a selectable marker in the vector sequence, such as a drug selectable marker, Cyanobacterial cells containing the gene deletion can be readily isolated, identified and characterized. Such selectable vector-based recombination methods need not be limited to targeting upstream and downstream flanking regions, but may also be targeted to internal sequences within a given gene, as long as that gene is rendered “non-functional,” as described herein.

The generation of deletions or mutations can also be accomplished using antisense-based technology. For instance, Cyanobacteria have been shown to contain natural regulatory events that rely on antisense regulation, such as a 177-nt ncRNA that is transcribed in antisense to the central portion of an iron-regulated transcript and blocks its accumulation through extensive base pairing (see, e.g., Duhring, et al., Proc. Natl. Acad. Sci. USA 103:7054-7058, 2006), as well as a alr1690 mRNA that overlaps with, and is complementary to, the complete furA gene, which acts as an antisense RNA (α-furA RNA) interfering with furA transcript translation (see, e.g., Hernandez et al., Journal of Molecular Biology 355:325-334, 2006). Thus, the incorporation of antisense molecules targeted to genes involved in glycogen biosynthesis or storage or lipid biosynthesis would be similarly expected to negatively regulate the expression of these genes, rendering them “non-functional,” as described herein.

As used herein, antisense molecules encompass both single and double-stranded polynucleotides comprising a strand having a sequence that is complementary to a target coding strand of a gene or mRNA. Thus, antisense molecules include both single-stranded antisense oligonucleotides and double-stranded siRNA molecules.

In certain aspects, modified photosynthetic microorganisms, e.g., Cyanobacteria, that may be used in the systems and methods of the present invention may be prepared by: (i) modifying a photosynthetic microorganism so that it expresses a reduced amount of one or more genes associated with a glycogen biosynthesis or storage pathway and/or expresses an increased amount of one or more polynucleotides encoding a polypeptide associated with a glycogen breakdown pathway or secretion of a glycogen precursor; and (ii) introducing into the photosynthetic microorganism one or more polynucleotides encoding one or more enzymes associated with lipid biosynthesis, secretion of glucose, isobutanol and/or isopentanol biosynthesis, 4-hydroxybutyrate and/or 1,4-butanediol biosynthesis, or polyamine intermediate biosynthesis, as described elsewhere herein, and/or (iii) introducing into the photosynthetic microorganism one or more polynucleotide regulatory elements (e.g., promoters, enhancers) that increase or otherwise regulate expression of one or more endogenous enzymes associated with lipid biosynthesis, secretion of glucose, isobutanol and/or isopentanol biosynthesis, 4-hydroxybutyrate and/or 1,4-butanediol biosynthesis, or polyamine intermediate biosynthesis; and/or (iv) modifying a photosynthetic microorganism so that it expresses a reduced amount and/or a reduced-function mutant of one or more selected genes/polypeptides associated with lipid biosynthesis, as described herein. The methods may further comprise a step of: (v) selecting for photosynthetic microorganisms in which the one or more desired polynucleotides were successfully introduced, where the polyucleotides were, e.g., present in a vector the expressed a selectable marker, such as an antibiotic resistance gene. As one example, selection and isolation may include the use of antibiotic resistant markers known in the art (e.g., kanamycin, spectinomycin, and streptomycin).

Other modifications described herein may be produced using standard procedures and reagents, e.g., vectors, available in the art. Related methods are described in PCT Application No. WO 2010/075440, which is hereby incorporated by reference in its entirety.

### Methods of Producing Lipids

The systems and methods of the present invention may be used to produce lipids, such as fatty acids, triglycerides, alkanes/alkenes, fatty alcohols, and/or wax esters. Accordingly, the present invention provides methods of producing lipids comprising culturing any of the modified photosynthetic microorganisms described herein under stress conditions wherein the modified photosynthetic microorganism produces, secretes and/or accumulates (e.g., stores,) an increased amount of cellular lipid as compared to a corresponding wild-type or unmodified photosynthetic microorganism grown under said stress condition.

In one embodiment, the modified photosynthetic microorganism is a Cyanobacterium that produces or accumulates increased fatty acids relative to an unmodified or wild-type Cyanobacterium of the same species grown under said stress condition. In specific embodiments, the modified photosynthetic microorganism such as Cyanobacteria produces increased levels of particular fatty acids, such as C16:0 fatty acids. In certain embodiments, the modified photosynthetic microorganism is a Cyanobacterium that produces or accumulates increased wax esters relative to an unmodified or wild-type Cyanobacterium of the same species when grown under said stress condition. In particular embodiments, the modified photosynthetic microorganism is a Cyanobacterium that produces or accumulates increased triglycerides relative to an unmodified or wild-type Cyanobacterium of the same species when grown under said stress condition. In some embodiments, the modified photosynthetic microorganism is a Cyanobacterium that produces or accumulates increased alkanes and/or alkenes relative to an unmodified or wild-type Cyanobacterium of the same species when grown under said stress condition.

In certain embodiments, the one or more introduced polynucleotides are present in one or more expression constructs. In particular embodiments, the one or more expression constructs comprises one or more inducible promoters. In certain embodiments, the one or more expression constructs are stably integrated into the genome of said modified photosynthetic microorganism. In certain embodiments, the introduced polynucleotide encoding an introduced protein is present in an expression construct comprising a weak promoter under non-induced conditions. In certain embodiments, one or more of the introduced polynucleotides are codon-optimized for expression in a Cyanobacterium, e.g., a Synechococcus elongatus.

In particular embodiments, the photosynthetic microorganism is a Synechococcus elongatus, such as Synechococcus elongatus strain PCC7942 or a salt tolerant variant of Synechococcus elongatus strain PCC7942.

In particular embodiments, the photosynthetic microorganism is a Synechococcus sp. PCC 7002 or a Synechocystis sp. PCC6803.

In particular embodiments, the modified photosynthetic microorganisms are cultured under conditions suitable for inducing expression of the introduced polynucleotide(s), e.g., wherein the introduced polynucleotide(s) comprise an inducible promoter. Conditions and reagents suitable for inducing inducible promoters are known and available in the art. Also included are the use of auto-inductive systems, for example, where a metabolite represses expression of the introduced polynucleotide, and the use of that metabolite by the microorganism over time decreases its concentration and thus its repressive activities, thereby allowing increased expression of the polynucleotide sequence.

In certain embodiments, modified photosynthetic microorganisms, e.g., Cyanobacteria, are grown under conditions favorable for producing lipids, triglycerides and/or fatty acids. In particular embodiments, light intensity is between 100 and 2000 uE/m2/s, or between 200 and 1000 uE/m2/s. In particular embodiments, the pH range of culture media is between 7.0 and 10.0. In certain embodiments, CO₂ is injected into the culture apparatus to a level in the range of 1% to 10%. In particular embodiments, the range of CO₂ is between 2.5% and 5%. In certain embodiments, nutrient supplementation is performed during the linear phase of growth. Each of these conditions may be desirable for triglyceride production.

In certain embodiments, the modified photosynthetic microorganisms are cultured, at least for some time, under static growth conditions as opposed to shaking conditions. For example, the modified photosynthetic microorganisms may be cultured under static conditions prior to inducing expression of an introduced polynucleotide (e.g., acyl-ACP reductase, ACP, glycogen breakdown protein, ACCase, DGAT, fatty acyl-CoA synthetase, aldehyde dehydrogenase, alcohol dehydrogenase, aldehyde decarbonylase) and/or the modified photosynthetic microorganism may be cultured under static conditions while expression of an introduced polynucleotide is being induced, or during a portion of the time period during which expression on an introduced polynucleotide is being induced. Static growth conditions may be defined, for example, as growth without shaking or growth wherein the cells are shaken at less than or equal to 30 rpm or less than or equal to 50 rpm.

In certain embodiments, the modified photosynthetic microorganisms are cultured, at least for some time, in media supplemented with varying amounts of bicarbonate. For example, the modified photosynthetic microorganisms may be cultured with bicarbonate at 5, 10, 20, 50, 75, 100, 200, 300, 400, 500, 600, 700, 800, 900, 1000 mM bicarbonate prior to inducing expression of an introduced polynucleotide (e.g., acyl-ACP reductase, ACP, glycogen breakdown protein, ACCase, DGAT, fatty acyl-CoA synthetase, alcohol dehydrogenase, aldehyde dehydrogenase, aldehyde decarbonylase) and/or the modified photosynthetic microorganism may be cultured with aforementioned bicarbonate concentrations while expression of an introduced polynucleotide is being induced, or during a portion of the time period during which expression on an introduced polynucleotide is being induced.

In related embodiments, modified photosynthetic microorganisms and methods of the present invention may be used in the production of a biofuel or other specialty chemical. Thus, in particular embodiments, a method of producing a biofuel comprises culturing any of the modified photosynthetic microorganisms of the present invention under conditions wherein the modified photosynthetic microorganism accumulates an increased amount of total cellular lipid (e.g., fatty acid, wax ester, alkane/alkene, fatty alcohol, and/or triglyceride), as compared to a corresponding wild-type photosynthetic microorganism, obtaining the cellular lipid from said microorganism, and processing the obtained cellular lipid to produce a biofuel. In another embodiment, a method of producing a biofuel comprises processing lipids (e.g., fatty acids, wax esters, alkanes/alkenes, fatty alcohols, triglycerides) produced by a modified photosynthetic microorganism of the present invention to produce a biofuel. In particular embodiments, the modified photosynthetic microorganism is grown under stress conditions wherein it has reduced growth but maintains photosynthesis.

Methods of processing lipids from microorganisms to produce a biofuel or other specialty chemical, e.g., biodiesel, are known and available in the art. For example, triglycerides may be transesterified to produce biodiesel. Transesterification may be carried out by any one of the methods known in the art, such as alkali-, acid-, or lipase-catalysis (see, e.g., Singh et al. Recent Pat Biotechnol. 2008, 2(2):130-143). Various methods of transesterification utilize, for example, use of a batch reactor, a supercritical alcohol, an ultrasonic reactor, or microwave irradiation (Such methods are described, for example, in Jeong and Park, Appl Biochem Biotechnol. 2006, 131(1-3):668-679; Fukuda et al., Journal of Bioscience and Engineering. 2001, 92(5):405-416; Shah and Gupta, Chemistry Central Journal. 2008, 2(1):1-9; and Carrillo-Munoz et al., J Org Chem. 1996, 61(22):7746-7749). The biodiesel may be further processed or purified, e.g., by distillation, and/or a biodiesel stabilizer may be added to the biodiesel, as described, for example, in U.S. Patent Application Publication No. 2008/0282606.

### Polypeptides

Modified photosynthetic microorganisms of the present invention comprise one or more introduced or (over)expressed polypeptides, reduced expression and/or activity of one or more polypeptides, or a combination thereof. In particular embodiments, the photosynthetic microorganisms described herein have been modified to accumulate a reduced amount of glycogen as compared to a corresponding wild-type photosynthetic microorganism. As one example, such modified photosynthetic microorganism my comprise reduced expression and/or activity of one or more polypeptides associated with glycogen synthesis and/or glycogen storage. Alternatively or in combination with the above, such modified photosynthetic microorganism may comprise one or more introduced or overexpressed polypeptides associated with glycogen breakdown and/or secretion of glycogen precursors. Examples of such glycogen-associated polypeptides are described below.

These modified photosynthetic microorganism can optionally further comprise one or more introduced, expressed, or overexpressed lipid biosynthesis proteins, e.g., one or more proteins associated with fatty acid synthesis, triglyceride synthesis, alkane synthesis, wax ester synthesis, or other lipid synthesis pathway described herein. Certain aspects, however, may comprise reduced expression and/or activity of one or more selected lipid biosynthesis proteins, for instance, to shunt carbon away from one lipid and towards another lipid. Examples of polypeptides associated with lipid biosynthesis are also described below.

The modified photosynthetic microorganisms described herein can optionally comprise one or more introduced, expressed or overexpressed polypeptides associated with the secretion or synthesis of other carbon-containing compounds described herein, including polypeptides associated with the secretion of glucose and polypeptides associated with the synthesis of isobutanol, isopentanol, 4-hydroxybutyrate, 1,4-butanediol, and polyamines and intermediates thereof.

It is further understood that the compositions and methods of the present invention may be practiced using biologically active variants and/or fragments of any of these or other introduced or overexpressed polypeptides. As will be apparent, modified photosynthetic microorganisms of the present invention may comprise any combination of one or more of the additional modifications noted herein.

### Glycogen Synthesis, Storage, and Breakdown Proteins

The modified photosynthetic microorganisms of the present invention have reduced production and/or storage of glycogen. For instance, certain modified photosynthetic microorganisms described herein have reduced expression and/or activity of one or more polypeptides associated with a glycogen synthesis or storage pathway and/or increased expression of one or more polypeptides associated with a glycogen breakdown pathway, or a functional variant of fragment thereof. Also included are modifications that increase secretion of a glycogen precursor, for instance, by overexpressing one or more polypeptides associated with glycogen precursor secretion.

In various embodiments, modified photosynthetic microorganisms, e.g., Cyanobacteria, of the present invention have reduced expression of one or more polypeptides associated with glycogen synthesis and/or storage. In particular embodiments, these modified photosynthetic microorganisms have a mutated or deleted gene that encodes a polypeptide associated with glycogen synthesis and/or storage. In particular embodiments, these modified photosynthetic microorganisms comprise a vector that includes a portion of a mutated or deleted gene, e.g., a targeting vector used to generate a knockout or knockdown of one or more alleles of the mutated or deleted gene. In certain embodiments, these modified photosynthetic microorganisms comprise an antisense RNA or siRNA that binds to an mRNA expressed by a gene associated with glycogen synthesis and/or storage.

In certain embodiments, modified photosynthetic microorganisms, e.g., Cyanobacteria, of the present invention comprise one or more exogenous or introduced nucleic acids that encode a polypeptide having an activity associated with a glycogen breakdown or triglyceride or fatty acid biosynthesis, including but not limited to any of those described herein. In particular embodiments, the exogenous nucleic acid does not comprise a nucleic acid sequence that is native to the microorganism's genome. In particular embodiments, the exogenous nucleic acid comprises a nucleic acid sequence that is native to the microorganism's genome, but it has been introduced into the microorganism, e.g., in a vector or by molecular biology techniques, for example, to increase expression of the nucleic acid and/or its encoded polypeptide in the microorganism.

Glycogen Biosynthesis and Storage.

Glycogen is a polysaccharide of glucose, which functions as a means of carbon and energy storage in most cells, including animal and bacterial cells. More specifically, glycogen is a very large branched glucose homopolymer containing about 90% α-1,4-glucosidic linkages and 10% α-1,6 linkages. For bacteria in particular, the biosynthesis and storage of glycogen in the form of α-1,4-polyglucans represents an important strategy to cope with transient starvation conditions in the environment.

Glycogen biosynthesis involves the action of several enzymes. For instance, bacterial glycogen biosynthesis occurs generally through the following general steps: (1) formation of glucose-1-phosphate, catalyzed by phosphoglucomutase (Pgm), followed by (2) ADP-glucose synthesis from ATP and glucose 1-phosphate, catalyzed by glucose-1-phosphate adenylyltransferase (GlgC), followed by (3) transfer of the glucosyl moiety from ADP-glucose to a pre-existing α-1,4 glucan primer, catalyzed by glycogen synthase (GlgA). This latter step of glycogen synthesis typically occurs by utilizing ADP-glucose as the glucosyl donor for elongation of the α-1,4-glucosidic chain.

In bacteria, the main regulatory step in glycogen synthesis takes place at the level of ADP-glucose synthesis, or step (2) above, the reaction catalyzed by glucose-1-phosphate adenylyltransferase (GlgC), also known as ADP-glucose pyrophosphorylase (see, e.g., Ballicora et al., Microbiology and Molecular Biology Reviews 6:213-225, 2003). In contrast, the main regulatory step in mammalian glycogen synthesis occurs at the level of glycogen synthase. As shown herein, by altering the regulatory and/or other active components in the glycogen synthesis pathway of photosynthetic microorganisms such as Cyanobacteria, and thereby reducing the biosynthesis and storage of glycogen, the carbon that would have otherwise been stored as glycogen can be utilized by said photosynthetic microorganism to synthesize other carbon-containing storage molecules, such as lipids, fatty acids, and triglycerides.

Therefore, certain modified photosynthetic microorganisms, e.g., Cyanobacteria, of the present invention may comprise a mutation, deletion, or any other alteration that disrupts one or more of these steps (i.e., renders the one or more steps “non-functional” with respect to glycogen biosynthesis and/or storage), or alters any one or more of the enzymes directly involved in these steps, or the genes encoding them. As noted above, such modified photosynthetic microorganisms, e.g., Cyanobacteria, are typically capable of producing and/or accumulating an increased amount of lipids, such as fatty acids, as compared to a wild-type photosynthetic microorganism. Certain exemplary glycogen biosynthesis genes are described below.

Phosphoglucomutase Gene (Ppm).

In one embodiment, a modified photosynthetic microorganism, e.g., a Cyanobacteria, expresses a reduced amount of the phosphoglucomutase gene. In particular embodiments, it may comprise a mutation or deletion in the phosphoglucomutase gene, including any of its regulatory elements (e.g., promoters, enhancers, transcription factors, positive or negative regulatory proteins, etc.). Phosphoglucomutase (Pgm), encoded by the gene pgm, catalyzes the reversible transformation of glucose 1-phosphate into glucose 6-phosphate, typically via the enzyme-bound intermediate, glucose 1,6-biphosphate (see, e.g., Lu et al., Journal of Bacteriology 176:5847-5851, 1994). Although this reaction is reversible, the formation of glucose-6-phosphate is markedly favored.

However, typically when a large amount of glucose-6-phosphate is present, Pgm catalyzes the phosphorylation of the 1-carbon and the dephosphorylation of the c-carbon, resulting in glucose-1-phosphate. The resulting glucose-1-phosphate is then converted to UDP-glucose by a number of intermediate steps, including the catalytic activity of GlgC, which can then be added to a glycogen storage molecule by the activity of glycogen synthase, described below. Thus, under certain conditions, the Pgm enzyme plays an intermediary role in the biosynthesis and storage of glycogen.

The pgm gene is expressed in a wide variety of microorganisms, including most, if not all, Cyanobacteria. The pgm gene is also fairly conserved among Cyanobacteria, as can be appreciated upon comparison of SEQ ID NOs:24 (S. elongatus PCC7942), 25 (Synechocystis sp. PCC6803), and 26 (Synechococcus sp. WH8102), which provide the polynucleotide sequences of various pgm genes from Cyanobacteria.

Deletion of the pgm gene in Cyanobacteria, such as Synechococcus, has been demonstrated herein for the first time to reduce the accumulation of glycogen in said Cyanobacteria, and also to increase the production of other carbon-containing compounds, such as lipids, biofuels, other specialty chemicals, and precursors thereof.

Glucose-1-Phosphate Adenylyltransferase (glgC).

In one embodiment, a modified photosynthetic microorganism, e.g., a Cyanobacteria, expresses a reduced amount of a glucose-1-phosphate adenylyltransferase (glgC) gene. In certain embodiments, it may comprise a mutation or deletion in the glgC gene, including any of its regulatory elements. The enzyme encoded by the glgC gene (e.g., EC 2.7.7.27) participates generally in starch, glycogen and sucrose metabolism by catalyzing the following chemical reaction:

ATP+alpha-D-glucose 1-phosphate [symbol] diphosphate+ADP-glucose

Thus, the two substrates of this enzyme are ATP and alpha-D-glucose 1-phosphate, whereas its two products are diphosphate and ADP-glucose. The glgC-encoded enzyme catalyzes the first committed and rate-limiting step in starch biosynthesis in plants and glycogen biosynthesis in bacteria. It is the enzymatic site for regulation of storage polysaccharide accumulation in plants and bacteria, being allosterically activated or inhibited by metabolites of energy flux.

The enzyme encoded by the glgC gene belongs to a family of transferases, specifically those transferases that transfer phosphorus-containing nucleotide groups (i.e., nucleotidyl-transferases). The systematic name of this enzyme class is typically referred to as ATP:alpha-D-glucose-1-phosphate adenylyltransferase. Other names in common use include ADP glucose pyrophosphorylase, glucose 1-phosphate adenylyltransferase, adenosine diphosphate glucose pyrophosphorylase, adenosine diphosphoglucose pyrophosphorylase, ADP-glucose pyrophosphorylase, ADP-glucose synthase, ADP-glucose synthetase, ADPG pyrophosphorylase, and ADP:alpha-D-glucose-1-phosphate adenylyltransferase.

The glgC gene is expressed in a wide variety of plants and bacteria, including most, if not all, Cyanobacteria. The glgC gene is also fairly conserved among Cyanobacteria, as can be appreciated upon comparison of SEQ ID NOs:27 (S. elongatus PCC7942), 28 (Synechocystis sp. PCC6803), 29 (Synechococcus sp. PCC 7002), 30 (Synechococcus sp. WH8102), 31 (Synechococcus sp. RCC 307), 32 (Trichodesmium erythraeum IMS 101), 33 (Anabaena varibilis), and 34 (Nostoc sp. PCC 7120), which describe the polynucleotide sequences of various glgC genes from Cyanobacteria.

Deletion of the glgC gene in Cyanobacteria, such as Synechococcus, has been demonstrated herein for the first time to reduce the accumulation of glycogen in said Cyanobacteria, and also to increase the production of other carbon-containing compounds, such as lipids and fatty acids.

Glycogen Synthase (glgA).

In one embodiment, a modified photosynthetic microorganism, e.g., a Cyanobacteria, expresses a reduced amount of a glycogen synthase gene. In particular embodiments, it may comprise a deletion or mutation in the glycogen synthase gene, including any of is regulatory elements. Glycogen synthase (GlgA), also known as UDP-glucose-glycogen glucosyltransferase, is a glycosyltransferase enzyme that catalyses the reaction of UDP-glucose and (1,4-α-D-glucosyl)ₙ to yield UDP and (1,4-α-D-glucosyl)ₙ₊₁. Glycogen synthase is an α-retaining glucosyltransferase that uses ADP-glucose to incorporate additional glucose monomers onto the growing glycogen polymer. Essentially, GlgA catalyzes the final step of converting excess glucose residues one by one into a polymeric chain for storage as glycogen.

Classically, glycogen synthases, or α-1,4-glucan synthases, have been divided into two families, animal/fungal glycogen synthases and bacterial/plant starch synthases, according to differences in sequence, sugar donor specificity and regulatory mechanisms. However, detailed sequence analysis, predicted secondary structure comparisons, and threading analysis show that these two families are structurally related and that some domains of animal/fungal synthases were acquired to meet the particular regulatory requirements of those cell types.

Crystal structures have been established for certain bacterial glycogen synthases (see, e.g., Buschiazzo et al., The EMBO Journal 23, 3196-3205, 2004). These structures show that reported glycogen synthase folds into two Rossmann-fold domains organized as in glycogen phosphorlyase and other glycosyltransferases of the glycosyltransferases superfamily, with a deep fissure between both domains that includes the catalytic center. The core of the N-terminal domain of this glycogen synthase consists of a nine-stranded, predominantly parallel, central β-sheet flanked on both sides by seven α-helices. The C-terminal domain (residues 271-456) shows a similar fold with a six-stranded parallel β-sheet and nine α-helices. The last α-helix of this domain undergoes a kink at position 457-460, with the final 17 residues of the protein (461-477) crossing over to the N-terminal domain and continuing as α-helix, a typical feature of glycosyltransferase enzymes.

These structures also show that the overall fold and the active site architecture of glycogen synthase are remarkably similar to those of glycogen phosphorylase, the latter playing a central role in the mobilization of carbohydrate reserves, indicating a common catalytic mechanism and comparable substrate-binding properties. In contrast to glycogen phosphorylase, however, glycogen synthase has a much wider catalytic cleft, which is predicted to undergo an important interdomain ‘closure’ movement during the catalytic cycle.

Crystal structures have been established for certain GlgA enzymes (see, e.g., Jin et al., EMBO J 24:694-704, 2005, incorporated by reference). These studies show that the N-terminal catalytic domain of GlgA resembles a dinucleotide-binding Rossmann fold and the C-terminal domain adopts a left-handed parallel beta helix that is involved in cooperative allosteric regulation and a unique oligomerization. Also, communication between the regulator-binding sites and the active site involves several distinct regions of the enzyme, including the N-terminus, the glucose-1-phosphate-binding site, and the ATP-binding site.

The glgA gene is expressed in a wide variety of cells, including animal, plant, fungal, and bacterial cells, including most, if not all, Cyanobacteria. The glgA gene is also fairly conserved among Cyanobacteria, as can be appreciated upon comparison of SEQ ID NOs:35 (S. elongatus PCC7942), 36 (Synechocystis sp. PCC6803), 37 (Synechococcus sp. PCC 7002), 38 (Synechococcus sp. WH8102), 39 (Synechococcus sp. RCC 307), 40 (Trichodesmium erythraeum IMS 101), 41 (Anabaena variabilis), and 42 (Nostoc sp. PCC 7120), which describe the polynucleotide sequences of various glgA genes from Cyanobacteria.

Glycogen Breakdown.

In certain embodiments, a modified photosynthetic microorganism of the present invention expresses an increased amount of one or more polypeptides associated with a glycogen breakdown pathway. In particular embodiments, said one or more polypeptides include a glycogen phosphorylase (GlgP), glycogen isoamylase (GlgX), glucanotransferase (MalQ), phosphoglucomutase (Pgm), glucokinase (Glk), and/or phosphoglucose isomerase (Pgi), or a functional fragment or variant thereof, including, for example, those provided in SEQ ID NOs:68, 70, 72, 73, 83 or 85. Examples of additional Pgm polypeptide sequences useful according to the present invention are provided in SEQ ID NOs:74, 76, 77, 79, and 81. As noted above, Pgm, Glk, and Pgi are bidirectional enzymes that can promote glycogen synthesis or breakdown depending on conditions.

### Lipid Biosynthesis Proteins

In various embodiments, and further to modifications that reduce production and/or storage of glycogen, certain modified photosynthetic microorganisms of the present invention further comprise one or more introduced or overexpressed lipid biosynthesis proteins, e.g., polypeptide(s) having an activity associated with lipid biosynthesis, including triglyceride, fatty acid, fatty alcohol, alkane/alkene, and/or wax ester biosynthesis, In some instances, a modified photosynthetic microorganism may comprise reduced expression and/or activity of one or more selected lipid biosynthesis proteins. Certain of these proteins are described in greater detail below.

In particular embodiments, an exogenous nucleic acid encoding a lipid biosynthesis protein does not comprise a nucleic acid sequence that is native to the microorganism's genome. In some embodiments, an exogenous nucleic acid comprises a nucleic acid sequence that is native to the microorganism's genome, but it has been introduced into the microorganism, e.g., in a vector or by molecular biology techniques, for example, to increase expression of the nucleic acid and/or its encoded polypeptide in the microorganism. In certain embodiments, the expression of a native or endogenous nucleic acid and its corresponding protein can be increased by introducing a heterologous promoter upstream of the native gene. As noted above, lipid biosynthesis proteins can be involved in triglyceride biosynthesis, fatty acid synthesis, wax ester synthesis, fatty alcohol synthesis, alkane synthesis, or any combination thereof.

Triglyceride Biosynthesis.

Triglycerides, or triacylglycerols (TAGs), consist primarily of glycerol esterified with three fatty acids, and yield more energy upon oxidation than either carbohydrates or proteins. Triglycerides provide an important mechanism of energy storage for most eukaryotic microorganisms. In mammals, TAGs are synthesized and stored in several cell types, including adipocytes and hepatocytes (Bell et al. Annu. Rev. Biochem. 49:459-487, 1980) (herein incorporated by reference). In plants, TAG production is mainly important for the generation of seed oils.

In contrast to eukaryotes, the observation of triglyceride production in prokaryotes has been limited to certain actinomycetes, such as members of the genera Mycobacterium, Nocardia, Rhodococcus and Streptomyces, in addition to certain members of the genus Acinetobacter. In certain Actinomycetes species, triglycerides may accumulate to nearly 80% of the dry cell weight, but accumulate to only about 15% of the dry cell weight in Acinetobacter. In general, triglycerides are stored in spherical lipid bodies, with quantities and diameters depending on the respective species, growth stage, and cultivation conditions. For example, cells of Rhodococcus opacus and Streptomyces lividans contain only few TAGs when cultivated in complex media with a high content of carbon and nitrogen; however, the lipid content and the number of TAG bodies increase drastically when the cells are cultivated in mineral salt medium with a low nitrogen-to-carbon ratio, yielding a maximum in the late stationary growth phase. At this stage, cells can be almost completely filled with lipid bodies exhibiting diameters ranging from 50 to 400 nm. One example is R. opacus PD630, in which lipids can reach more than 70% of the total cellular dry weight.

In bacteria, TAG formation typically starts with the docking of a diacylglycerol acyltransferase enzyme to the plasma membrane, followed by formation of small lipid droplets (SLDs). These SLDs are only some nanometers in diameter and remain associated with the membrane-docked enzyme. In this phase of lipid accumulation, SLDs typically form an emulsive, oleogenous layer at the plasma membrane. During prolonged lipid synthesis, SLDs leave the membrane-associated acyltransferase and conglomerate to membrane-bound lipid prebodies. These lipid prebodies reach distinct sizes, e.g., about 200 nm in A. calcoaceticus and about 300 nm in R. opacus, before they lose contact with the membrane and are released into the cytoplasm. Free and membrane-bound lipid prebodies correspond to the lipid domains occurring in the cytoplasm and at the cell wall, as observed in M. smegmatis during fluorescence microscopy and also confirmed in R. opacus PD630 and A. calcoaceticus ADP1 (see, e.g., Christensen et al., Mol. Microbiol. 31:1561-1572, 1999; and Wältermann et al., Mol. Microbiol. 55:750-763, 2005). Inside the lipid prebodies, SLDs coalesce with each other to form the homogenous lipid core found in mature lipid bodies, which often appear opaque in electron microscopy. The compositions and structures of bacterial TAGs vary considerably depending on the microorganism and on the carbon source. In addition, unusual acyl moieties, such as phenyldecanoic acid and 4,8,12 trimethyl tridecanoic acid, may also contribute to the structural diversity of bacterial TAGs (see, e.g., Alvarez et al., Appl Microbiol Biotechnol. 60:367-76, 2002).

As with eukaryotes, the main function of TAGs in prokaryotes is to serve as a storage compound for energy and carbon. TAGs, however, may provide other functions in prokaryotes. For example, lipid bodies may act as a deposit for toxic or useless fatty acids formed during growth on recalcitrant carbon sources, which must be excluded from the plasma membrane and phospholipid (PL) biosynthesis. Furthermore, many TAG-accumulating bacteria are ubiquitous in soil, and in this habitat, water deficiency causing dehydration is a frequent environmental stress. Storage of evaporation-resistant lipids might be a strategy to maintain a basic water supply, since oxidation of the hydrocarbon chains of the lipids under conditions of dehydration would generate considerable amounts of water. Cyanobacteria such as Synechococcus, however, do not produce triglycerides, because these microorganisms lack the enzymes necessary for triglyceride biosynthesis.

Triglycerides are synthesized from fatty acids and glycerol. As one mechanism of triglyceride (TAG) synthesis, sequential acylation of glycerol-3-phosphate via the “Kennedy Pathway” leads to the formation of phosphatidate. Phosphatidate is then dephosphorylated by the enzyme phosphatidate phosphatase to yield 1,2 diacylglycerol (DAG). Using DAG as a substrate, at least three different classes of enzymes are capable of mediating TAG formation. As one example, an enzyme having diacylglycerol acyltransferase (DGAT) activity catalyzes the acylation of DAG using acyl-CoA as a substrate. Essentially, DGAT enzymes combine acyl-CoA with 1,2 diacylglycerol molecule to form a TAG. As an alternative, Acyl-CoA-independent TAG synthesis may be mediated by a phospholipid:DAG acyltransferase found in yeast and plants, which uses phospholipids as acyl donors for DAG esterification. Third, TAG synthesis in animals and plants may be mediated by a DAG-DAG-transacylase, which uses DAG as both an acyl donor and acceptor, yielding TAG and monoacylglycerol.

Since wild-type Cyanobacteria do not typically encode the enzymes necessary for triglyceride synthesis, such as the enzymes having diacylglycerol acyltransferase activity, embodiments of the present invention include genetically modified Cyanobacteria that comprise polynucleotides encoding one or more enzymes having a diacylglycerol acyltransferase activity, optionally in combination with one or more enzymes having a fatty acyl-CoA synthetase activity.

Moreover, since triglycerides are typically formed from fatty acids, the level of fatty acid biosynthesis in a cell may limit the production of triglycerides. Increasing the level of fatty acid biosynthesis may, therefore, allow increased production of triglycerides. As discussed below, acetyl-CoA carboxylase catalyzes the commitment step to fatty acid biosynthesis. Thus, certain embodiments of the present invention include Cyanobacterium, and methods of use thereof, comprising polynucleotides that encode one or more enzymes having Acetyl-CoA carboxylase activity to increase fatty acid biosynthesis and lipid production, in addition to one or more enzymes having diacylglycerol acyltransferase activity and one or more enzymes having fatty acyl-CoA synthetase activity, to catalyze triglyceride production.

Fatty Acid Biosynthesis.

Fatty acids are a group of negatively charged, linear hydrocarbon chains of various length and various degrees of oxidation states. The negative charge is located at a carboxyl end group and is typically deprotonated at physiological pH values (pK ˜2-3). The length of the fatty acid ‘tail’ determines its water solubility (or rather insolubility) and amphipathic characteristics. Fatty acids are components of phospholipids and sphingolipids, which form part of biological membranes, as well as triglycerides, which are primarily used as energy storage molecules inside cells.

Fatty acids are formed from acetyl-CoA and malonyl-CoA precursors. Malonyl-CoA is a carboxylated form of acetyl-CoA, and contains a 3-carbon dicarboxylic acid, malonate, bound to Coenzyme A. Acetyl-CoA carboxylase catalyzes the 2-step reaction by which acetyl-CoA is carboxylated to form malonyl-CoA. In particular, malonate is formed from acetyl-CoA by the addition of CO₂ using the biotin cofactor of the enzyme acetyl-CoA carboxylase.

Fatty acid synthase (FAS) carries out the chain elongation steps of fatty acid biosynthesis. FAS is a large multienzyme complex. In mammals, FAS contains two subunits, each containing multiple enzyme activities. In bacteria and plants, individual proteins, which associate into a large complex, catalyze the individual steps of the synthesis scheme. For example, in bacteria and plants, the acyl carrier protein is a smaller, independent protein.

Fatty acid synthesis starts with acetyl-CoA, and the chain grows from the “tail end” so that carbon 1 and the alpha-carbon of the complete fatty acid are added last. The first reaction is the transfer of an acetyl group to a pantothenate group of acyl carrier protein (ACP), a region of the large mammalian fatty acid synthase (FAS) protein. In this reaction, acetyl CoA is added to a cysteine —SH group of the condensing enzyme (CE) domain: acetyl CoA+CE-cys-SH->acetyl-cys-CE+CoASH. Mechanistically, this is a two step process, in which the group is first transferred to the ACP (acyl carrier peptide), and then to the cysteine —SH group of the condensing enzyme domain.

In the second reaction, malonyl CoA is added to the ACP sulfhydryl group: malonyl CoA+ACP-SH->malonyl ACP+CoASH. This —SH group is part of a phosphopantethenic acid prosthetic group of the ACP.

In the third reaction, the acetyl group is transferred to the malonyl group with the release of carbon dioxide: malonyl ACP+acetyl-cys-CE->beta-ketobutyryl-ACP+CO₂.

In the fourth reaction, the keto group is reduced to a hydroxyl group by the beta-ketoacyl reductase activity: beta-ketobutyryl-ACP+NADPH+H⁺->beta-hydroxybutyryl-ACP+NAD⁺.

In the fifth reaction, the beta-hydroxybutyryl-ACP is dehydrated to form a trans-monounsaturated fatty acyl group by the beta-hydroxyacyl dehydratase activity: beta-hydroxybutyryl-ACP->2-butenoyl-ACP+H₂O.

In the sixth reaction, the double bond is reduced by NADPH, yielding a saturated fatty acyl group two carbons longer than the initial one (an acetyl group was converted to a butyryl group in this case): 2-butenoyl-ACP+NADPH+H⁺->butyryl-ACP+NADP⁺. The butyryl group is then transferred from the ACP sulfhydryl group to the CE sulfhydryl: butyryl-ACP+CE-cys-SH->ACP-SH+butyryl-cys-CE. This step is catalyzed by the same transferase activity utilized previously for the original acetyl group. The butyryl group is now ready to condense with a new malonyl group (third reaction above) to repeat the process. When the fatty acyl group becomes 16 carbons long, a thioesterase activity hydrolyses it, forming free palmitate: palmitoyl-ACP+H₂O->palmitate+ACP-SH. Fatty acid molecules can undergo further modification, such as elongation and/or desaturation.

Modified photosynthetic microorganisms, e.g., Cyanobacteria, may comprise one or more exogenous polynucleotides encoding any of the above polypeptides or enzymes involved in fatty acid synthesis. In particular embodiments, the enzyme is an acetyl-CoA carboxylase or a variant or functional fragment thereof.

Wax Ester Synthesis.

Wax esters are esters of a fatty acid and a long-chain alcohol. These neutral lipids are composed of aliphatic alcohols and acids, with both moieties usually long-chain (e.g., C₁₆ and C₁₈) or very-long-chain (C₂₀ and longer) carbon structures, though medium-chain-containing wax esters are included (e.g., C₁₀, C₁₂ and C₁₄). Wax esters have diverse biological functions in bacteria, insects, mammals, and terrestrial plants and are also important substrates for a variety of industrial applications. Various types of wax ester are widely used in the manufacture of fine chemicals such as cosmetics, candles, printing inks, lubricants, coating stuffs, and others.

In certain microorganisms, such as Acinetobacter, the pathway for wax ester synthesis of Acinetobacter spp. has been assumed to start from acyl coenzyme A (acyl-CoA), which is then reduced to the corresponding alcohol via acyl-CoA reductase and aldehyde reductase. In other microorganisms, for example, wax ester biosynthesis involves elongation of saturated C₁₆ and C₁₈ fatty acyl-CoAs to very-long-chain fatty acid wax precursors between 24 and 34 carbons in length, and their subsequent modification by either the alkane-forming (decarbonylation) or the alcohol-forming (acyl reduction) pathway (see Li et al., Plant Physiology 148:97-107, 2008).

In certain aspects, wax ester synthesis can occur via the acyl-ACP=>acyl aldehyde pathway. In this pathway, acyl-ACP reductase overexpression increases conversion of acyl-ACP into acyl aldehydes, alcohol dehydrogenase overexpression then increases conversion of acyl aldehydes into fatty alcohols, and DGAT overexpression cooperatively increases conversion of the fatty alcohols into their corresponding wax esters. Modified photosynthetic microorganisms, e.g., Cyanobacteria, may therefore comprise one or more exogenous polynucleotides encoding any of the above polypeptides or enzymes involved in wax ester synthesis.

Acyl-ACP Reductases.

Acyl-ACP reductases (or acyl-ACP dehydrogenases) are members of the reductase or short-chain dehydrogenase family, and are key enzymes of the type II fatty acid synthesis (FAS) system. Among other potential catalytic activities, an “acyl-ACP reductase” or “acyl-ACP dehydrogenase” as used herein is capable of catalyzing the conversion (reduction) of acyl-ACP to an acyl aldehyde (see Schirmer et al., supra) and the concomitant oxidation of NAD(P)H to NADP⁺. In some embodiments, the acyl-ACP reductase preferentially interacts with acyl-ACP, and does not interact significantly with acyl-CoA, i.e., it does not significantly catalyze the conversion of acyl-CoA to acyl aldehyde.

Acyl-ACP reductases can be derived from a variety of plants and bacteria, included photosynthetic microorganisms such as Cyanobacteria. One exemplary acyl-ACP reductase is encoded by orf1594 of Synechococcus elongatus PCC7942 (see SEQ ID NOs:1 and 2 for the polynucleotide and polypeptide sequences, respectively). Another exemplary acyl-ACP reductase is encoded by orfsll0209 of Synechocystis sp. PCC6803 (SEQ ID NOs:3 and 4 for the polynucleotide and polypeptide sequences, respectively). Hence, in certain embodiments, an acyl-ACP reductase comprises or consists of the exemplary polypeptide sequence of SEQ ID NO:2, encoded by orf1594 from Synechococcus elongatus PCC7942, including active variants or fragments thereof. In some embodiments, an acyl-ACP reductase comprises or consists of the exemplary polypeptide sequence of SEQ ID NO:4, encoded by orfsll0209 from Synechocystis sp. PCC6803, including active variants or fragments thereof.

Introduced or overexpressed acyl-ACP reductases can increase production of a variety of lipids, including fatty acids, triglycerides, alkanes, fatty alcohols, and wax esters. For example, and further to modifications that reduce production and/or storage of glycogen, increased acyl-ACP expression can increase the production of fatty acids, optionally in combination with increased expression of an aldehyde dehydrogenase, and also optionally in combination with reduced expression of an endogenous aldehyde decarbonylase. As another example, increased acyl-ACP expression in combination with DGAT can increase the production of triglycerides, optionally in combination with increased expression of an aldehyde dehydrogenase (to increase fatty acids, a precursor to triglycerides) and/or reduced expression of an endogenous aldehyde aldehyde decarbonylase (to shunt carbon away from other carbon-containing compounds such as alkanes). As a further example, increased acyl-ACP expression in combination with DGAT and an alcohol dehydrogenase can increase production of wax esters, optionally in combination with reduced expression of an aldehyde decarbonylase. As another example, increased increased acyl-ACP expression in combination with increased aldehyde decarbonylase expression can increase production of alkanes, optionally in combination with decreased expression of an alcohol dehydrogenase and/or decreased expression of an aldehyde dehydrogenase (to shunt carbon away from fatty alcohols and fatty acids towards alkanes). Other combinations will be apparent to persons skilled in the art based on the description provided herein.

Acyl Carrier Proteins.

Embodiments of the present invention optionally include one or more exogenous (e.g., recombinantly introduced) or overexpressed ACP proteins. These proteins play crucial roles in fatty acid synthesis. Fatty acid synthesis in bacteria, including Cyanobacteria, is carried out by highly conserved enzymes of the type II fatty acid synthase system (FAS II; consisting of about 19 genes) in a sequential, regulated manner. Acyl carrier protein (ACP) plays a central role in this process by carrying all the intermediates as thioesters attached to the terminus of its 4′-phosphopantetheine prosthetic group (ACP-thioesters). Apo-ACP, the product of acp gene, is typically activated by a phosphopantetheinyl transferease (PPT) such as the acyl carrier protein synthase (AcpS) type found in E. coli or the Sfp (surfactin type) PTT as characterized in Bacillus subtilis. Cyanobacteria posses an Sfp-like PPT, which is understood to act in both primary and secondary metabolism. Embodiments of the present invention therefore include overexpression of PPTs such as AcpS and/or Sfp-type PPTs in combination with overexpression of cognate ACP encoding genes, such as ACP.

The ACP-thioesters are substrates for all of the enzymes of the FAS II system. The end product of fatty acid synthesis is a long acyl chain typically consisting of about 14-18 carbons attached to ACP by a thioester bond.

At least three enzymes of the FAS II system in other bacteria can be subject to feedback inhibition by acyl-ACPs: 1) the ACCase complex—a heterotetramer of the AccABCD genes that catalyzes the production of malonyl-coA, the first step in the pathway; 2) the product of the FabH gene (β-ketoacyl-ACP synthase III), which catalyzes the condensation of acetyl-CoA with malonyl-ACP; and 3) the product of the FabI gene (enoyl-ACP reductase), which catalyzes the final elongation step in each round of elongation. Certain proteins such as acyl-ACP reductase are capable of increasing fatty acid production in photosynthetic bacteria such as Cyanobacteria, and it is believed that overexpression of ACP in combination with this protein and possibly other biosynthesis proteins will further increases fatty acid production in such strains.

An ACP can be derived from a variety of eukaryotic organisms, microorganisms (e.g., bacteria, fungi), or plants. In certain embodiments, an ACP polynucleotide sequence and its corresponding polypeptide sequence are derived from Cyanobacteria such as Synechococcus. In certain embodiments, ACPs can be derived from plants such as spinach. SEQ ID NOS:5-12 provide the nucleotide and polypeptide sequences of exemplary bacterial ACPs from Synechococcus and Acinetobacter, and SEQ ID NOS:13-14 provide the same for an exemplary plant ACP from Spinacia oleracea (spinach). SEQ ID NOS:5 and 6 derive from Synechococcus elongatus PCC7942, and SEQ ID NOS:7-12 derive from Acinetobacter sp. ADP1. Thus, in certain embodiments, an acyl carrier protein (ACP) comprises or consists of the exemplary ACP polypeptide sequences include SEQ ID NO:6 from Synechococcus elongatus PCC7942, SEQ ID NOS:8, 10, and 12 from Acinetobacter sp. ADP1, or SEQ ID NO:14 from Spinacia oleracea.

Examples of prokaryotic microorganisms having an ACP include certain actinomycetes, a group of Gram-positive bacteria with high G+C ratio, such as those from the representative genera Actinomyces, Arthrobacter, Corynebacterium, Frankia, Micrococcus, Mocrimonospora, Mycobacterium, Nocardia, Propionibacterium, Rhodococcus and Streptomyces. Particular examples of actinomycetes that have one or more genes encoding an ACP activity include, for example, Mycobacterium tuberculosis, M. avium, M. smegmatis, Micromonospora echinospora, Rhodococcus opacus, R. ruber, and Streptomyces lividans. Additional examples of prokaryotic microorganisms that encode one or more enzymes having an ACP activity include members of the genera Acinetobacter, such as A. calcoaceticus, A. baumanii, A. baylii, and members of the generua Alcanivorax. In certain embodiments, an ACP gene or enzyme is isolated from Acinetobacter baylii sp. ADP1, a gram-negative triglyceride forming prokaryote.

Acyl ACP Synthases (Aas).

Acyl-ACP synthetases (Aas) catalyze the ATP-dependent acylation of the thiol of acyl carrier protein (ACP) with fatty acids, including those fatty acids having chain lengths from about C4 to C18. In Cyanobacteria, among other functions, Aas enzymes not only directly incorporate exogenous fatty acids from the culture medium into other lipids, but also play a role in the recycling of acyl chains from lipid membranes. Deletion of Aas in cyanobacteria can lead to secretion of free fatty acids into the culture medium. See, e.g., Kaczmarzyk and Fulda, Plant Physiology 152:1598-1610, 2010.

Certain embodiments may overexpress one or more Aas polypeptides described herein and known in the art. According to one non-limiting theory, overexpression of Aas in combination with overexpression of ACP leads to increased TAG production in DGAT-expressing strains, for example, by boosting acyl-ACP levels. Overexpression of Aas in optional combination with overexpression of ACP may likewise increase wax ester formation, for example, when combined with overexpression of one or more alcohol dehydrogenase(s) and wax ester synthase(s), such as a bi-functional DGAT. Certain embodiments therefore include modified photosynthetic microorganisms comprising overexpressed Aas polypeptide(s), optionally in combination with overexpressed ACP polypeptide(s), especially when combined with overexpression of alcohol dehydrogenase, acyl-ACP reductase (e.g., orf1594), and wax ester synthase (e.g., aDGAT).

Examples of bacterial Aas enzymes include those derived from E. coli, Acinetobacter, and Vibrio sp. such as V. harveyi (see, e.g., Shanklin, Protein Expression and Purification. 18:355-360, 2000; Jiang et al., Biochemistry. 45:10008-10019, 2006). SEQ ID NOS:43 and 44, respectively, provide the nucleotide and polypeptide sequences of an exemplary Aas from Synechococcus elongatus PCC 7942 (0918).

In specific embodiments, the Aas is derived from the same microorganism as the overexpressed ACP, DGAT, and/or the TES, if any one of these polypeptides is employed in combination with an Aas. Accordingly, certain embodiments include Aas sequences from any of the microorganisms described herein for deriving a DGAT or TES, including, for example, various animals (e.g., mammals, fruit flies, nematodes), plants, parasites, and fungi (e.g., yeast such as S. cerevisiae and Schizosaccharomyces pombe). Examples of prokaryotic microorganisms include certain actinomycetes, a group of Gram-positive bacteria with high G+C ratio, such as those from the representative genera Actinomyces, Arthrobacter, Corynebacterium, Frankia, Micrococcus, Mocrimonospora, Mycobacterium, Nocardia, Propionibacterium, Rhodococcus and Streptomyces. Particular examples of actinomycetes that have one or more genes encoding an Aas activity include, for example, Mycobacterium tuberculosis, M. avium, M. smegmatis, Micromonospora echinospora, Rhodococcus opacus, R. ruber, and Streptomyces lividans. Additional examples of prokaryotic microorganisms that encode one or more enzymes having an Aas activity include members of the genera Acinetobacter, such as A. calcoaceticus, A. baumanii, A. baylii, and members of the generua Alcanivorax. In certain embodiments, an Aas gene or enzyme is isolated from Acinetobacter baylii sp. ADP1, a gram-negative triglyceride forming prokaryote.

According to one non-limiting theory, an endogenous aldehyde dehydrogenase may be acting on the excess acyl-aldehydes generated by overexpressed orf1594 and converting them to free fatty acids. The normal role of such a dehydrogenase might involve removing or otherwise dealing with damaged lipids. In this scenario, it is then likely that the Aas gene product recycles these free fatty acids by ligating them to ACP. Accordingly, reducing or eliminating expression of the Aas gene product might ultimately increase production of fatty acids, by reducing or preventing their transfer to ACP. Hence, certain aspects include mutations (e.g., genomic) such as point mutations, deletions, and insertions that reduce or eliminate the expression or enzymatic activity of one or more endogenous acyl-ACP synthetases (or synthases). Also included are full or partial deletions of an endogenous gene encoding an Aas protein.

Phosphatidate Phosphatase (PAP).

As used herein, a “phosphatidate phosphatase” or “phosphatidic acid phosphatase” gene of the present invention includes any polynucleotide sequence encoding amino acids, such as protein, polypeptide or peptide, obtainable from any cell source, which demonstrates the ability to catalyze the dephosphorylation of phosphatidate (PtdOH) under enzyme reactive conditions, yielding diacylglycerol (DAG) and inorganic phosphate, and further includes any naturally-occurring or non-naturally occurring variants of a phosphatidate phosphatase sequence having such ability.

Phosphatidate phosphatases (PAP, 3-sn-phosphatidate phosphohydrolase) catalyze the dephosphorylation of phosphatidate (PtdOH), yielding diacylglycerol (DAG) and inorganic phosphate. This enzyme belongs to the family of hydrolases, specifically those acting on phosphoric monoester bonds. The systematic name of this enzyme class is 3-sn-phosphatidate phosphohydrolase. Other names in common use include phosphatic acid phosphatase, acid phosphatidyl phosphatase, and phosphatic acid phosphohydrolase. This enzyme participates in at least 4 metabolic pathways: glycerolipid metabolism, glycerophospholipid metabolism, ether lipid metabolism, and sphingolipid metabolism.

PAP enzymes have roles in both the synthesis of phospholipids and triacylglycerol through its product diacylglycerol, as well as the generation or degradation of lipid-signaling molecules in eukaryotic cells. PAP enzymes are typically classified as either Mg²⁺-dependent (referred to as PAP1 enzymes) or Mg²⁺-independent (PAP2 or lipid phosphate phosphatase (LPP) enzymes) with respect to their cofactor requirement for catalytic activity. In both yeast and mammalian systems, PAP2 enzymes are known to be involved in lipid signaling. By contrast, PAP1 enzymes, such as those found in Saccharomyces cerevisiae, play a role in de novo lipid synthesis (Han, et al. J Biol Chem. 281:9210-9218, 2006), thereby revealing that the two types of PAP are responsible for different physiological functions.

In both yeast and higher eukaryotic cells, the PAP reaction is the committed step in the synthesis of the storage lipid triacylglycerol (TAG), which is formed from PtdOH through the intermediate DAG. The reaction product DAG is also used in the synthesis of the membrane phospholipids phosphatidylcholine (PtdCho) and phosphatidylethanolamine. The substrate PtdOH is used for the synthesis of all membrane phospholipids (and the derivative inositol-containing sphingolipids) through the intermediate CDP-DAG. Thus, regulation of PAP activity might govern whether cells make storage lipids and phospholipids through DAG or phospholipids through CDP-DAG. In addition, PAP is involved in the transcriptional regulation of phospholipid synthesis.

PAP1 enzymes have been purified and characterized from the membrane and cytosolic fractions of yeast, including a gene (Pah1, formerly known as Smp2) been identified to encode a PAP1 enzyme is S. cerevisiae. The Pah1-encoded PAP1 enzyme is found in the cytosolic and membrane fractions of the cell, and its association with the membrane is peripheral in nature. As expected from the multiple forms of PAP1 that have been purified from yeast, pah1Δ mutants still contain PAP1 activity, indicating the presence of an additional gene or genes encoding enzymes having PAP1 activity.

Analysis of mutants lacking the Pah1-encoded PAP1 has provided evidence that this enzyme generates the DAG used for lipid synthesis. Cells containing the pah1Δ mutation accumulate PtdOH and have reduced amounts of DAG and its acylated derivative TAG. Phospholipid synthesis predominates over the synthesis of TAG in exponentially growing yeast, whereas TAG synthesis predominates over the synthesis of phospholipids in the stationary phase of growth. The effects of the pah1Δ mutation on TAG content are most evident in the stationary phase. For example, stationary phase cells devoid of the Pah1 gene show a reduction of >90% in TAG content. Likewise, the pah1Δ mutation shows the most marked effects on phospholipid composition (e.g. the consequent reduction in PtdCho content) in the exponential phase of growth. The importance of the Pah1-encoded PAP1 enzyme to cell physiology is further emphasized because of its role in the transcriptional regulation of phospholipid synthesis.

The requirement of Mg²⁺ ions as a cofactor for PAP enzymes is correlated with the catalytic motifs that govern the phosphatase reactions of these enzymes. For example, the Pah1-encoded PAP1 enzyme has a DxDxT (SEQ ID NO:198) catalytic motif within a haloacid dehalogenase (HAD)-like domain (“x” is any amino acid). This motif is found in a superfamily of Mg²⁺-dependent phosphatase enzymes, and its first aspartate residue is responsible for binding the phosphate moiety in the phosphatase reaction. By contrast, the DPP1- and LPP1-encoded PAP2 enzymes contain a three-domain lipid phosphatase motif that is localized to the hydrophilic surface of the membrane. This catalytic motif, which comprises the consensus sequences KxxxxxxRP (domain 1) (SEQ ID NO:116), PSGH (domain 2) (SEQ ID NO:117), and SRxxxxxHxxxD (domain 3) (SEQ ID NO:118), is shared by a superfamily of lipid phosphatases that do not require Mg²⁺ ions for activity. The conserved arginine residue in domain 1 and the conserved histidine residues in domains 2 and 3 may be essential for the catalytic activity of PAP2 enzymes. Accordingly, a phosphatidate phosphatase polypeptide may comprise one or more of the above-described catalytic motifs.

A polynucleotide encoding a polypeptide having a phosphatidate phosphatase enzymatic activity may be obtained from any organism having a suitable, endogenous phosphatidate phosphatase gene. Examples of organisms that may be used to obtain a phosphatidate phosphatase encoding polynucleotide sequence include, but are not limited to, Homo sapiens, Mus musculus, Rattus norvegicus, Bos taurus, Drosophila melanogaster, Arabidopsis thaliana, Magnaporthe grisea, Saccharomyces cerevisiae, Schizosaccharomyces pombe, Cryptococcus neoformans, and Bacillus pumilus, among others. Specific examples of PAP enzymes include Pah1 from S. cerevisiae, PgpB from E. coli, and PAP from PCC6803.

In certain embodiments, a phosphatidate phosphatase polypeptide comprises or consists of a polypeptide sequence set forth in SEQ ID NO:131, or a fragment or variant thereof. In particular embodiments, a phosphatidate phosphatase is encoded by a polynucleotide sequence set forth in SEQ ID NO:129 or SEQ ID NO:130, or a fragment or variant thereof. SEQ ID NO:131 is the sequence of Saccharomyces cerevisiae phosphatidate phosphatase (yPah1), and SEQ ID NO:129 is a codon-optimized for expression in Cyanobacteria sequence that encodes yPah1. In certain embodiments, the polypeptide sequence of the PAP is encoded by the E. coli PgpB gene, and/or the PAP gene from Synechocystis sp. PCC6803.

Thioesterases (TES).

Certain embodiment include one or more exogenous or overexpressed thioesterase enzymes, optionally in combination with at least one of an introduced ACP enzyme, an introduced Aas enzyme, or both. For instance, one embodiment relates to the use an introduced ACP and/or Aas to increase the growth and/or fatty acid production of a free fatty acid producing TES strain, such as a TesA strain or a FatB strain (i.e., a strain having an introduced TesA or FatB). Thioesterases, as referred to herein, exhibit esterase activity (splitting of an ester into acid and alcohol, in the presence of water) specifically at a thiol group. Fatty acids are often attached to cofactor molecules, such as coenzyme A (CoA) and acyl carrier protein (ACP), by thioester linkages during the process of de novo fatty acid synthesis. Certain embodiments employ thioesterases having acyl-ACP thioesterase activity, acyl-CoA thioesterase activity, or both activities. Examples of thioesterases having both activities (i.e., acyl-ACP/acyl-CoA thioesterases) include TesA and related embodiments. In certain embodiments, a selected thioesterase has acyl-ACP thioesterase activity but not acyl-CoA thioesterase activity. Examples of thioesterases having only acyl-ACP thioesterase activity include the FatB thioesterases and related embodiments.

Certain thioesterases have both thioesterase activity and lysophospholipase activity. Specific examples of thioesterases include TesA, TesB, and related embodiments. Certain embodiments may employ periplasmically-localized or cytoplasmically-localized enzymes that thioesterase activity, such as E. coli TesA or E. coli TesB. For instance, wild-type TesA, being localized to the periplasm, is normally used to hydrolyze thioester linkages of fatty acid-ACP (acyl-ACP) or fatty acid-CoA (acyl-CoA) compounds scavenged from the environment. A mutant thioesterase, PldC (referred to interchangeably as PldC/*TesA or *TesA), is not exported to the periplasm due to deletion of an N-terminal amino acid sequence required for proper transport of TesA from the cytoplasm to the periplasm. This deletion results in a cytoplasmic-localized PldC(*TesA) protein that has access to endogenous acyl-ACP and acyl-CoA intermediates. Other mutations or deletions in the N-terminal region of TesA can be used to achieve the same result, i.e., a cytoplasmic TesA.

Overexpressed PldC(*TesA) results in hydrolysis of acyl groups from endogenous acyl-ACP and acyl-CoA molecules. Cells expressing PldC(*TesA) must channel additional cellular carbon and energy to maintain production of acyl-ACP and acyl-coA molecules, which are required for membrane lipid synthesis. Thus, PldC(*TesA) expression results in a net increase in total cellular lipid content. For instance, PldC(*TesA) expressed alone in Synechococcus doubles the total lipid content from 10% of biomass to 20% of biomass, a result that can be further increased by combining *TesA or related molecules with an introduced ACP and/or an introduced Aas. Hence, certain embodiments employ an exogenous or overexpressed cytoplasmic TesA (such as *TesA) in combination with an exogenous or overexpressed ACP, an exogenous or overexpressed Aas, or both.

Certain thioesterases have thioesterase activity only, i.e., they have little or no lysophospholipase activity. Examples of these thioesterases include enzymes of the FatB family. FatB encoded enzymes typically hydrolyze saturated C14-C18 ACPs, preferentially 16:0 ACP, but they can also hydrolyze 18:1 ACP. The production of medium chain (C8-C12) fatty acids in plants or seeds such as those of Cuphea spp. often results of FatB enzymes that have chain length specificities for medium chain fatty acyl-ACPs. These medium chain FatB thioesterases are present in many species with medium-chain fatty acids in their oil, including, for example, California bay laurel, coconut, and elm, among others. Hence, FatB sequences may be derived from these and other organisms. Particular examples include plant FatB acyl-ACP thioesterases such as C8, C12, C14, and C16 FatB thioesterases.

In certain embodiments, the TES is a FatB polypeptide, such as a C8, C12, C14, C16, or C18 FatB. Specific examples of FatB thioesterases include the Cuphea hookeriana C8/C10 FatB thioesterase, the Umbellularia californica C12 FatB1 thioesterase, the Cinnamomum camphora C14 FatB1 thioesterase, and the Cuphea hookeriana C16 FatB1 thioesterase. In specific embodiments, the thioesterase is a Cuphea hookeriana C8/C10 FatB, comprising the amino acid sequence of SEQ ID NO:108 (full-length protein) or SEQ ID NO:109 (mature protein without signal sequence). In particular embodiments, the thioesterase is a Umbellularia californica C12 FatB1, comprising the amino acid sequence of SEQ ID NO:110 (full-length protein) or SEQ ID NO:111 (mature protein without signal sequence). In certain embodiments, the thioesterase is a Cinnamomum camphora C14 FatB1, comprising the amino acid sequence of SEQ ID NO:112 (full-length protein) or SEQ ID NO:113 (mature protein without signal sequence). In particular embodiments, the thioesterase is a Cuphea hookeriana C16 FatB1, comprising the amino acid sequence of SEQ ID NO:114 (full-length protein) or SEQ ID NO:115 (mature protein without signal sequence), or a fragment or variant thereof.

Lipases and Phospholipases.

In various embodiments, modified photosynthetic microorganisms, e.g., Cyanobacteria, of the present invention further comprise one or more exogenous or introduced nucleic acids that encode a polypeptide having a lipase or phospholipase activity, or a fragment or variant thereof. Lipases, including phospholipases, lysophospholipases, thioesterases, and enzymes having one, two, or all three of these activities, typically catalyze the hydrolysis of ester chemical bonds in lipid substrates. Without wishing to be bound by any one theory, in certain exemplary embodiments the expression of one or more phospholipases can generate fatty acids from membrane lipids, which may then be used by the ACP and/or Aas to make acyl-ACPs. These acyl-ACPs, for example, can then feed into the triglyceride synthesis pathways, thereby increasing triglyceride (TAG) production.

A phospholipase is an enzyme that hydrolyzes phospholipids into fatty acids and other lipophilic substances. There are four major classes, termed A, B, C and D distinguished by what type of reaction they catalyze. Phospholipase A1 cleaves the SN-1 acyl chain, while Phospholipase A2 cleaves the SN-2 acyl chain, releasing arachidonic acid. Phospholipase B cleaves both SN-1 and SN-2 acyl chains, and is also known as a lysophospholipase. Phospholipase C cleaves before the phosphate, releasing diacylglycerol and a phosphate-containing head group. Phospholipases C play a central role in signal transduction, releasing the second messenger, inositol triphosphate. Phospholipase D cleaves after the phosphate, releasing phosphatidic acid and an alcohol. Types C and D are considered phosphodiesterases. In various embodiments of the present invention, one or more phospholipase from any one of these classes may be used, alone or in any combination.

As noted above, phospholipases (PLA1,2) act on phospholipids of different kinds including phosphatidyl glycerol, the major phospholipid in Cyanobacteria, by cleaving the acyl chains off the sn1 or sn2 positions (carbon 1 or 2 on the glycerol backbone); some are selective for sn1 or sn2, others act on both. Lysophospholipases act on lysophospholipids, which can be the product of phospholipases or on lysophosphatidic acid, a normal intermediate of the de novo phosphatidic acid synthesis pathway, e.g., 1-acyl-DAG-3-phosphate.

Merely by way of non-limiting theory, it is understood that in certain embodiments, phospholipases and/or lysophospholipases can cleave off acyl chains from phospholipids or lysophospholipids and thus deregulate the normal recycling of the lipid membranes, including both cell membrane and thylakoid membranes, which then leads to accumulation of free fatty acids (FFAs). In certain embodiments (e.g., TesA strains), these FFAs may accumulate extracellularly. In other embodiments (e.g., ACP and/or Aas over-expressing microorganisms), FFAs can be converted into acyl-ACPs by acyl ACP synthase (Aas) in a strain that also over-expresses ACP. In specific embodiments (e.g., DGAT-containing microorganisms), these acyl-ACPs can then serve as substrates for DGAT to make TAGs.

In other embodiments, phospholipases can be over-expressed to generate lyshophospholipids and acyl chains. The lysophospholipids can then serve as substrates for a lysophospholipase, which cleaves off the remaining acyl chain. In some embodiments, these acyl chains can either accumulate as FFAs, or in other embodiments may serve as substrates of Acyl ACP synthase (Aas) to generate acyl-ACPs, which can then be used by DGAT to make TAGs.

Particular examples of phospholipase C enzymes include those derived from eukaryotes such as mammals and parasites, in addition to those derived from bacteria. Examples include phosphoinositide phospholipase C (EC 3.1.4.11), the main form found in eukaryotes, especially mammals, the zinc-dependent phospholipase C family of bacterial enzymes (EC 3.1.4.3) that includes alpha toxins, phosphatidylinositol diacylglycerol-lyase (EC 4.6.1.13), a related bacterial enzyme, and glycosylphosphatidylinositol diacylglycerol-lyase (EC 4.6.1.14), a trypanosomal enzyme.

In particular embodiments, the present invention contemplates using a lysophospholipase. A lysophospholipase is an enzyme that catalyzes the chemical reaction:

2-lysophosphatidic acid+H₂O [symbol] glycerol-3-phosphate+a carboxylate

Thus, the two substrates of this enzyme are 2-lysophosphatidylcholine and H₂O, whereas its two products are glycerophosphocholine and carboxylate.

Lysophospholipase are members of the hydrolase family, specifically those acting on carboxylic ester bonds. Lysophospholipases participate in glycerophospholipid metabolism. Examples of lysophospholipases include, but are not limited to, 2-Lysophosphatidylcholine acylhydrolase, Lecithinase B, Lysolecithinase, Phospholipase B, Lysophosphatidase, Lecitholipase, Phosphatidase B, Lysophosphatidylcholine hydrolase, Lysophospholipase A1, Lysophospholipase L1 (TesA), Lysophopholipase L2, TesB, Lysophospholipase transacylase, Neuropathy target esterase, NTE, NTE-LysoPLA, NTE-lysophospholipase, and Vu Patatin 1 protein. In particular embodiments, lysophospholipases utilized according to the present invention are derived from a bacteria, e.g., E. coli, or a plant. Any of these lysophospholipases may be used according to various embodiments of the present invention.

Certain lysophospholipases, such as Lysophospholipase L1 (also referred to as PldC or TesA) are periplasmically-localized or cytoplasmically-localized enzymes that have both lysophospholipase and thioesterase activity, as described above. Hence, certain thioesterases such as TesA can also be characterized as lysophospholipases. A mutant lysophospholipase described herein, PldC(*TesA), is not exported to the periplasm due to deletion of an N-terminal amino acid sequence required for proper transport of TesA from the cytoplasm to the periplasm. This results in a cytoplasmic-localized PldC(*TesA) protein that has access to endogenous acyl-ACP and acyl-CoA intermediates. Overexpressed PldC(*TesA) results in hydrolysis of acyl groups from endogenous acyl-ACP and acyl-CoA molecules. Cells expressing PldC(*TesA) must channel additional cellular carbon and energy to maintain production of acyl-ACP and acyl-coA molecules, which are required for membrane lipid synthesis. Thus, PldC(*TesA) expression results in a net increase in cellular lipid content. As described herein, PldC(*TesA) is expressed in Synechococcus lipid content doubles from 10% of biomass to 20% of biomass.

In certain embodiments of the present invention, lysophospholipases utilized according to the present invention have both phospholipase and thioesterase activities. Examples of lysophospholipases that have both activities include, e.g., Lysophospholipase L1 (TesA), such as E. coli Lysophospholipase L1, as well as fragments and variants thereof, including those described in the paragraph above. As a phospholipase, certain embodiments may employ TesA variants having only lysophospholipase activity, including variants with reduced or no thioesterase activity.

Additional non-limiting examples of phospholipases include phospholipase A1 (PldA) from Acinetobacter sp. ADP1, phospholipase A (PldA) from E. coli, phospholipase from Streptomyces coelicolorA3(2), phospholipase A2 (PLA2-α) from Arabidopsis thaliana; phospholipase A1/triacylglycerol lipase (DAD1; Defective Anther Dehiscence 1) from Arabidopsis thaliana, chloroplast DONGLE from Arabidopsis thaliana, patatin-like protein from Arabidopsis thaliana, and patatin from Anabaena variabilis ATCC 29413. Additional non-limiting examples of lysophospholipases include phospholipase B (Plb1p) from Saccharomyces cerevisiae S288c, phospholipase B (Plb2p) from Saccharomyces cerevisiae S288c, ACIAD1057 (tesA homolog) from Acinetobacter ADP1, ACIAD1943 lysophospholipase from Acinetobacter ADP1, and a lysophospholipase (YP_{—}702320; RHA1_ro02357) from Rhodococcus.

In particular embodiments, the encoded phospholipase comprises or consists of a Lysophospholipase L1 (TesA), Lysophospholipase L2, TesB, or Vu patatin 1 protein, or a homolog, fragment, or variant thereof. In certain embodiments, the Lysophospholipase L1 (TesA), Lysophospholipase L2, or TesB is a bacterial Lysophospholipase L1 (TesA), Lysophospholipase L2, or TesB, such as an E. coli Lysophospholipase L1 (TesA) having the wild-type sequence set forth in SEQ ID NO:133, an E. coli Lysophospholipase L2 having the wild-type sequence set forth in SEQ ID NO:137, or an E. coli TesB having the wild-type sequence set forth in SEQ ID NO:134. In particular embodiment, the Vu patatin 1 protein has the wild-type sequence set forth in SEQ ID NO:138.

In particular embodiments, the phospholipase is modified such that it localizes predominantly to the cytoplasm instead of the periplasm. For example, the phospholipase may have a deletion or mutation in a region associated with periplasmic localization. In particular embodiments, the phospholipase variant is derived from Lysophospholipase L1 (TesA) or TesB. In certain embodiments, the Lysophospholipase L1 (TesA) or TesB variant is a bacterial Lysophospholipase L1 (TesA) or TesB variant, such as a cytoplasmic E. coli Lysophospholipase L1 (PldC(*TesA)) variant having the sequence set forth in SEQ ID NO:121.

Additional examples of phospholipase polypeptide sequences include phospholipase A1 (PldA) from Acinetobacter sp. ADP1 (SEQ ID NO:157), phospholipase A (PldA) from E. coli (SEQ ID NO:158), phospholipase from Streptomyces coelicolor A3(2) (SEQ ID NO:159), phospholipase A2 (PLA2-α) from Arabidopsis thaliana (SEQ ID NO:160). phospholipase A1/triacylglycerol lipase (DAD1; Defective Anther Dehiscence 1) from Arabidopsis thaliana (SEQ ID NO:161), chloroplast DONGLE from Arabidopsis thaliana (SEQ ID NO:162), patatin-like protein from Arabidopsis thaliana (SEQ ID NO:163), and patatin from Anabaena variabilis ATCC 29413 (SEQ ID NO:164). Additional non-limiting examples of lysophospholipase polypeptide sequences include phospholipase B (Plb1p) from Saccharomyces cerevisiae S288c (SEQ ID NO:165), phospholipase B (Plb2p) from Saccharomyces cerevisiae S288c (SEQ ID NO:166), ACIAD1057 (TesA homolog) from Acinetobacter ADP1 (SEQ ID NO:167), ACIAD1943 lysophospholipase from Acinetobacter ADP1 (SEQ ID NO:168), and a lysophospholipase (YP_{—}702320; RHA1_ro02357) from Rhodococcus (SEQ ID NO:169).

Triacvlglycerol (TAG) Hydrolases.

Certain embodiments relate to the use of exogenous or overexpressed TAG hydrolases (or TAG lipases) to increase production of TAGs in a TAG-producing strain. For instance, specific embodiments may utilize a TAG hydrolase in combination with a DGAT, and optionally a TES. These embodiments may then further utilize an ACP, an Aas, or both, any of the lipid biosynthesis proteins described herein, and/or any of the modifications to glycogen production and storage described herein. Hence, as noted above, TAG hydrolases may be used in TAG-producing strains (e.g., DGAT-expressing strains) with or without an ACP or Aas.

TAG hydrolases are carboxylesterases that are typically specific for insoluble long chain fatty acid TAGs. Carboxylesterases catalyze the chemical reaction:

carboxylic ester+H₂O [symbol] alcohol+carboxylate

Thus, the two substrates of this enzyme are carboxylic ester and H₂O, whereas its two products are alcohol and carboxylate. According to one non-limiting theory, it is understood that TAG hydrolase expression (or overexpression) in a TAG producing strain (e.g., DGAT/ACP, DGAT/Aas, DGAT/ACP/Aas) releases acyl chains to not only increase accumulation of free fatty acids (FFA), but also increase the amount of free 1,2 diacylglycerol (DAG). This free DAG then serves as a substrate for DGAT, and thereby allows increased TAG production, especially in the presence of over-expressed ACP, Aas, or both. Accordingly, certain embodiments employing a TAG hydrolase produce increased amounts of TAG, relative, for example, to a DGAT only-expressing microorganism. In specific embodiments, the TAG hydrolase is specific for TAG and not DAG, i.e., it preferentially acts on TAG relative to DAG.

Non-limiting examples of TAG hydrolases include SDP1 (SUGAR-DEPENDENT1) triacylglycerol lipase from Arabidopsis thaliana (SEQ ID NO:170), ACIAD1335 from Acinetobacter sp. ADP1 (SEQ ID NO:171), TG14P from S. cerevisiae (SEQ ID NO:172), and RHA1_ro04722 (YP_{—}704665) TAG lipase from Rhodococcus (SEQ ID NO:173). Additional putative lipases/esterases from Rhodococcus include RHA1_ro01602 lipase/esterase (see SEQ ID NOs:156 and 174 for polynucleotide and polypeptide sequence, respectively), and RHA1_ro06856 lipase/esterase (see SEQ ID NOs:119 and 120 for polynucleotide and polypeptide sequence, respectively).

Acetyl CoA Carboxylases (ACCase).

Embodiments of the present invention optionally include one or more exogenous (e.g., recombinantly introduced) or overexpressed ACCase proteins. As used herein, an “acetyl CoA carboxylase” gene of the present invention includes any polynucleotide sequence encoding amino acids, such as protein, polypeptide or peptide, obtainable from any cell source, which demonstrates the ability to catalyze the carboxylation of acetyl-CoA to produce malonyl-CoA under enzyme reactive conditions, and further includes any naturally-occurring or non-naturally occurring variants of an acetyl-CoA carboxylase sequence having such ability.

Acetyl-CoA carboxylase (ACCase) is a biotin-dependent enzyme that catalyses the irreversible carboxylation of acetyl-CoA to produce malonyl-CoA through its two catalytic activities, biotin carboxylase (BC) and carboxyltransferase (CT). The biotin carboxylase (BC) domain catalyzes the first step of the reaction: the carboxylation of the biotin prosthetic group that is covalently linked to the biotin carboxyl carrier protein (BCCP) domain. In the second step of the reaction, the carboxyltransferase (CT) domain catalyzes the transfer of the carboxyl group from (carboxy) biotin to acetyl-CoA. Formation of malonyl-CoA by acetyl-CoA carboxylase (ACCase) represents the commitment step for fatty acid synthesis, because malonyl-CoA has no metabolic role other than serving as a precursor to fatty acids. Because of this reason, acetyl-CoA carboxylase represents a pivotal enzyme in the synthesis of fatty acids.

In most prokaryotes, ACCase is a multi-subunit enzyme, whereas in most eukaryotes it is a large, multi-domain enzyme. In yeast, the crystal structure of the CT domain of yeast ACCase has been determined at 2.7 A resolution (Zhang et al., Science, 299:2064-2067 (2003). This structure contains two domains, which share the same backbone fold. This fold belongs to the crotonase/ClpP family of proteins, with a b-b-a superhelix. The CT domain contains many insertions on its surface, which are important for the dimerization of ACCase. The active site of the enzyme is located at the dimer interface.

Although Cyanobacteria, such as Synechococcus, express a native ACCase enzyme, these bacteria typically do not produce or accumulate significant amounts of fatty acids. For example, Synechococcus in the wild accumulates fatty acids in the form of lipid membranes to a total of about 4% by dry weight.

Given the role of ACCase in the commitment step of fatty acid biosynthesis, embodiments of the present invention include methods of increasing the production of fatty acid biosynthesis, and, thus, lipid production, in Cyanobacteria by introducing one or more polynucleotides that encode an ACCase enzyme that is exogenous to the Cyanobacterium's native genome. Embodiments of the present invention also include a modified Cyanobacterium, and compositions comprising said Cyanobacterium, comprising one or more polynucleotides that encode an ACCase enzyme that is exogenous to the Cyanobacterium's native genome.

A polynucleotide encoding an ACCase enzyme may be isolated or obtained from any organism, such as any prokaryotic or eukaryotic organism that contains an endogenous ACCase gene. Examples of eukaryotic organisms having an ACCase gene are well-known in the art, and include various animals (e.g., mammals, fruit flies, nematodes), plants, parasites, and fungi (e.g., yeast such as S. cerevisiae and Schizosaccharomyces pombe). In certain embodiments, the ACCase encoding polynucleotide sequences are obtained from Synechococcus sp. PCC7002.

Examples of prokaryotic organisms that may be utilized to obtain a polynucleotide encoding an enzyme having ACCase activity include, but are not limited to, Escherichia coli, Legionella pneumophila, Listeria monocytogenes, Streptococcus pneumoniae, Bacillus subtilis, Ruminococcus obeum ATCC 29174, marine gamma proteobacterium HTCC2080, Roseovarius sp. HTCC2601, Oceanicola granulosus HTCC2516, Bacteroides caccae ATCC 43185, Vibrio alginolyticus 12G01, Pseudoalteromonas tunicata D2, Marinobacter sp. ELB17, marine gamma proteobacterium HTCC2143, Roseobacter sp. SK209-2-6, Oceanicola batsensis HTCC2597, Rhizobium leguminosarum bv. trifolii WSM1325, Nitrobacter sp. Nb-311A, Chloroflexus aggregans DSM 9485, Chlorobaculum parvum, Chloroherpeton thalassium, Acinetobacter baumannii, Geobacillus, and Stenotrophomonas maltophilia, among others.

In certain embodiments, an acetyl-CoA carboxylase (ACCase) polypeptide comprises or consists of a polypeptide sequence set forth in any of SEQ ID NOs:55, 45, 46, 47, 48 or 49, or a fragment or variant thereof. In particular embodiments, an ACCase polypeptide is encoded by a polynucleotide sequence set forth in any of SEQ ID NOs:56, 57, 50, 51, 52, 53 or 54, or a fragment or variant thereof. SEQ ID NO:55 is the sequence of Saccharomyces cerevisiae acetyl-CoA carboxylase (yAcc1); and SEQ ID NO:56 is a codon-optimized for expression in Cyanobacteria sequence that encodes yAcc1. SEQ ID NO:45 is Synechococcus sp. PCC 7002 AccA; SEQ ID NO:46 is Synechococcus sp. PCC 7002 AccB; SEQ ID NO:47 is Synechococcus sp. PCC 7002 AccC; and SEQ ID NO:48 is Synechococcus sp. PCC 7002 AccD. SEQ ID NO:50 encodes Synechococcus sp. PCC 7002 AccA; SEQ ID NO:51 encodes Synechococcus sp. PCC 7002 AccB; SEQ ID NO:52 encodes Synechococcus sp. PCC 7002 AccC; and SEQ ID NO:53 encodes Synechococcus sp. PCC 7002 AccD. SEQ ID NO:49 is a T. aestivum ACCase; and SEQ ID NO:54 encodes this Triticum aestivum ACCase.

Diacylglycerol Acyltransferases (DGAT).

As used herein, a “diacylglycerol acyltransferase” (DGAT) gene of the present invention includes any polynucleotide sequence encoding amino acids, such as protein, polypeptide or peptide, obtainable from any cell source, which demonstrates the ability to catalyze the production of triacylglycerol from 1,2-diacylglycerol and fatty acyl substrates under enzyme reactive conditions, in addition to any naturally-occurring (e.g., allelic variants, orthologs) or non-naturally occurring variants of a diacylglycerol acyltransferase sequence having such ability. DGAT genes of the present invention also include polynucleotide sequences that encode bi-functional proteins, such as those bi-functional proteins that exhibit a DGAT activity as well as a CoA:fatty alcohol acyltransferase activity, e.g., a wax ester synthesis (WS) activity, as often found in many TAG producing bacteria.

Diacylglycerol acyltransferases (DGATs) are members of the O-acyltransferase superfamily, which esterify either sterols or diacyglycerols in an oleoyl-CoA-dependent manner. DGAT in particular esterifies diacylglycerols, which reaction represents the final enzymatic step in the production of triacylglycerols in plants, fungi and mammals. Specifically, DGAT is responsible for transferring an acyl group from acyl-coenzyme-A to the sn-3 position of 1,2-diacylglycerol (DAG) to form triacylglycerol (TAG). DGAT is an integral membrane protein that has been generally described in Harwood (Biochem. Biophysics. Acta, 1301:7-56, 1996), Daum et al. (Yeast 16:1471-1510, 1998), and Coleman et al. (Annu. Rev. Nutr. 20:77-103, 2000) (each of which are herein incorporated by reference).

In plants and fungi, DGAT is associated with the membrane and lipid body fractions. In catalyzing TAGs, DGAT contributes mainly to the storage of carbon used as energy reserves. In animals, however, the role of DGAT is more complex. DGAT not only plays a role in lipoprotein assembly and the regulation of plasma triacylglycerol concentration (Bell, R. M., et al.), but participates as well in the regulation of diacylglycerol levels (Brindley, Biochemistry of Lipids, Lipoproteins and Membranes, eds. Vance, D. E. & Vance, J. E. (Elsevier, Amsterdam), 171-203; and Nishizuka, Science 258:607-614 (1992) (each of which are herein incorporated by reference)).

In eukaryotes, at least three independent DGAT gene families (DGAT1, DGAT2, and PDAT) have been described that encode proteins with the capacity to form TAG. Yeast contain all three of DGAT1, DGAT2, and PDAT, but the expression levels of these gene families varies during different phases of the life cycle (Dahlqvst, A., et al. Proc. Natl. Acad. Sci. USA 97:6487-6492 (2000) (herein incorporated by reference).

In prokaryotes, WS/DGAT from Acinetobacter calcoaceticus ADP1 represents the first identified member of a widespread class of bacterial wax ester and TAG biosynthesis enzymes. This enzyme comprises a putative membrane-spanning region but shows no sequence homology to the DGAT1 and DGAT2 families from eukaryotes. Under in vitro conditions, WS/DGAT shows a broad capability of utilizing a large variety of fatty alcohols, and even thiols as acceptors of the acyl moieties of various acyl-CoA thioesters. WS/DGAT acyltransferase enzymes exhibit extraordinarily broad substrate specificity. Genes for homologous acyltransferases have been found in almost all bacteria capable of accumulating neutral lipids, including, for example, Acinetobacter baylii, A. baumanii, and M. avium, and M. tuberculosis CDC1551, in which about 15 functional homologues are present (see, e.g., Daniel et al., J. Bacteriol. 186:5017-5030, 2004; and Kalscheuer et al., J. Biol. Chem. 287:8075-8082, 2003).

DGAT proteins may utilize a variety of acyl substrates in a host cell, including fatty acyl-CoA and fatty acyl-ACP molecules. In addition, the acyl substrates acted upon by DGAT enzymes may have varying carbon chain lengths and degrees of saturation, although DGAT may demonstrate preferential activity towards certain molecules.

Like other members of the eukaryotic O-acyltransferase superfamily, eukaryotic DGAT polypeptides typically contain a FYxDWWN (SEQ ID NO:15) heptapeptide retention motif, as well as a histidine (or tyrosine)-serine-phenylalanine (H/YSF) tripeptide motif, as described in Zhongmin et al. (Journal of Lipid Research, 42:1282-1291, 2001) (herein incorporated by reference). The highly conserved FYxDWWN (SEQ ID NO:15) is believed to be involved in fatty Acyl-CoA binding.

DGAT enzymes utilized according to the present invention may be isolated from any organism, including eukaryotic and prokaryotic organisms. Eukaryotic organisms having a DGAT gene are well-known in the art, and include various animals (e.g., mammals, fruit flies, nematodes), plants, parasites, and fungi (e.g., yeast such as S. cerevisiae and Schizosaccharomyces pombe). Examples of prokaryotic organisms include certain actinomycetes, a group of Gram-positive bacteria with high G+C ratio, such as those from the representative genera Actinomyces, Arthrobacter, Corynebacterium, Frankia, Micrococcus, Mocrimonospora, Mycobacterium, Nocardia, Propionibacterium, Rhodococcus and Streptomyces. Particular examples of actinomycetes that have one or more genes encoding a DGAT activity include, for example, Mycobacterium tuberculosis, M. avium, M. smegmatis, Micromonospora echinospora, Rhodococcus opacus, R. ruber, and Streptomyces lividans. Additional examples of prokaryotic organisms that encode one or more enzymes having a DGAT activity include members of the genera Acinetobacter, such as A. calcoaceticus, A. baumanii, A. baylii, and members of the generua Alcanivorax. In certain embodiments, a DGAT gene or enzyme is isolated from Acinetobacter baylii sp. ADP1, a gram-negative triglyceride forming prokaryote, which contains a well-characterized DGAT (AtfA).

In certain embodiments, the modified photosynthetic microorganisms of the present invention may comprise two or more polynucleotides that encode DGAT or a variant or fragment thereof. In particular embodiments, the two or more polynucleotides are identical or express the same DGAT. In certain embodiments, these two or more polynucleotides may be different or may encode two different DGAT polypeptides. For example, in one embodiment, one of the polynucleotides may encode ADGATd, while another polynucleotide may encode ScoDGAT. In particular embodiments, the following DGATs are coexpressed in modified photosynthetic microorganisms, e.g., Cyanobacteria, using one of the following double DGAT strains: ADGATd(NS1)::ADGATd(NS2); ADGATn(NS1)::ADGATn(NS2); ADGATn(NS1)::SDGAT(NS2); SDGAT(NS1)::ADGATn(NS2); SDGAT(NS1)::SDGAT(NS2). For the NS1 vector, pAM2291, EcoRI follows ATG and is part of the open reading frame (ORF). For the NS2 vector, pAM1579, EcoRI follows ATG and is part of the ORF. A DGAT having EcoRI nucleotides following ATG may be cloned in either pAM2291 or pAM1579; such a DGAT is referred to as ADGATd. Other embodiments utilize the vector, pAM2314FTrc3, which is an NS1 vector with Nde/BgIII sites, or the vector, pAM1579FTrc3, which is the NS2 vector with Nde/BgIII sites. A DGAT without EcoRI nucleotides may be cloned into either of these last two vectors. Such a DGAT is referred to as ADGATn. Modified photosynthetic microorganisms expressing different DGATs express TAGs having different fatty acid compositions. Accordingly, certain embodiments of the present invention contemplate expressing two or more different DGATs, in order to produce TAGs having varied fatty acid compositions.

In certain embodiments, a DGAT polypeptide comprises or consists of a polypeptide sequence set forth in any one of SEQ ID NOs:58, 59, 60 Or 61, or a fragment or variant thereof. SEQ ID NO:58 is the sequence of DGATn; SEQ ID NO:59 is the sequence of Streptomyces coelicolor DGAT (ScoDGAT or SDGAT); SEQ ID NO:60 is the sequence of Alcanivorax borkumensis DGAT (AboDGAT); and SEQ ID NO:61 is the sequence of DGATd. In certain embodiments of the present invention, a DGAT polypeptide is encoded by a polynucleotide sequence set forth in any one of SEQ ID NOs:62, 63, 64, 65 or 66, or a fragment or variant thereof. SEQ ID NO:62 is a codon-optimized for expression in Cyanobacteria sequence that encodes DGATn; SEQ ID NO:63 has homology to SEQ ID NO:62; SEQ ID NO:64 is a codon-optimized for expression in Cyanobacteria sequence that encodes ScoDGAT; SEQ ID NO:65 is a codon-optimized for expression in Cyanobacteria sequence that encodes AboDGAT; and SEQ ID NO:66 is a codon-optimized for expression in Cyanobacteria sequence that encodes DGATd.

Fatty Acyl-CoA Synthetases.

Certain embodiments relate to the use of overexpressed fatty acyl-CoA synthetases to increase activation of fatty acids, and thereby increase production of TAGs in a TAG-producing strain (e.g., a DGAT-expressing strain). For instance, specific embodiments may utilize an acyl-ACP reductase in combination with a fatty acyl-CoA synthetase and a DGAT. These embodiments may then further utilize an ACP, an ACCase, or both, and/or any of the modifications to glycogen production and storage or glycogen breakdown described herein.

Fatty acyl-CoA synthetases activate fatty acids for metabolism by catalyzing the formation of fatty acyl-CoA thioesters. Fatty acyl-CoA thioesters can then serve not only as substrates for beta-oxidation, at least in bacteria capable of growing on fatty acids as a sole source of carbon (e.g., E. coli, Salmonella), but also as acyl donors in phospholipid biosynthesis. Many fatty acyl-CoA synthetases are characterized by two highly conserved sequence elements, an ATP/AMP binding motif, which is common to enzymes that form an adenylated intermediate, and a fatty acid binding motif.

According to one non-limiting theory, certain embodiments may employ fatty acyl-CoA synthetases to increase activation of free fatty acids, which can then be incorporated into TAGs, mainly by the DGAT-expressing (and thus TAG-producing) photosynthetic microorganisms described herein. Hence, fatty acyl-CoA synthetases can be used in any of the embodiments described herein, such as those that produce increased levels of free fatty acids, where it is desirable to turn free fatty acids into TAGs. As noted above, these free fatty acids can then be activated by fatty acyl-CoA synthetases to generate acyl-CoA thioesters, which can then serve as substrates by DGAT to produce increased levels of TAGs.

One exemplary fatty acyl-CoA synthetase includes the FadD gene from E. coli (SEQ ID NOS:16 and 17 for nucleotide and polypeptide sequence, respectively), which encodes a fatty acyl-CoA synthetase having substrate specificity for medium and long chain fatty acids. Other exemplary fatty acyl-CoA synthetases include those derived from S. cerevisiae; Faa1p can use C12-C16 acyl-chains in vitro (see SEQ ID NOS:18 and 19 for nucleotide and polypeptide sequence, respectively), Faa2p shows a less restricted specificity ranging from C7-C17 (see SEQ ID NOS:20 and 21 for nucleotide and polypeptide sequence, respectively), and Faa3p, together with that of DGAT1, enhances lipid accumulation in the presence of exogenous fatty acids is S. cerevisiae (see SEQ ID NO:22 and 23 for nucleotide and polypeptide sequence, respectively). SEQ ID NO:22 is codon-optimized for expression in S. elongatus PCC7942. Hence, certain embodiments include fatty acyl-CoA synthetase polypeptides that comprises or consist of the Faa1p polypeptide sequence set forth in SEQ ID NO:19, the Faa2p polypeptide sequence set forth in SEQ ID NO:21, and/or the Faa3p polypeptide sequence set forth in SEQ ID NO:23, including active variants/fragments thereof.

Alcohol Dehydrogenases.

Embodiments of the present invention optionally include one or more introduced or expressed (e.g., overexpressed) alcohol dehydrogenases. Examples of alcohol dehydrogenases include those capable of using acyl or fatty aldehydes (e.g., one or more of nonyl-aldehyde, C₁₂, C₁₄, C₁₆, C₁₈, C₂₀ fatty aldehyde) as a substrate, and converting them into fatty alcohols. Specific examples include long-chain alcohol dehydrogenases, capable of using long-chain aldehydes (e.g., C₁₆, C₁₈, C₂₀) as substrates. In certain embodiments, the alcohol dehydrogenase is naturally-occurring or endogenous to the modified microorganism, and is sufficient to convert increased acyl aldehydes (produced by acyl-ACP reductase) into fatty alcohols, and thereby contribute to increased wax ester production and overall satisfactory growth characteristics. In certain embodiments, the alcohol dehydrogenase is derived from a microorganism that differs from the one being modified.

In these and related embodiments, expression or overexpression of an alcohol dehydrogenase may increase shunting of acyl aldehydes towards production of fatty alcohols, and away from production of other products such as alkanes, fatty acids, or triglycerides. When combined with one or more wax ester synthases, such as DGAT or other enzyme having wax ester synthase activity (e.g., the ability to convert fatty alcohols into wax esters), alcohol dehydrogenases may contribute to production of wax esters, optionally in combination with an overexpressed or introduced acyl-ACP reductase. They may also reduce accumulation of potentially toxic acyl aldehydes, and thereby improve growth characteristics of a modified microorganism.

Non-limiting examples of alcohol dehydrogenases include those encoded by slr1192 of Synechocystis sp. PCC6803 (SEQ ID NOS:104-105) and ACIAD3612 of Acinetobacter baylyi (SEQ ID NOS:106-107). Also included are homologs or paralogs thereof, functional equivalents thereof, and fragments or variants thereofs. Functional equivalents can include alcohol dehydrogenases with the ability to efficiently convert acyl aldehydes (e.g., C₆, C₈, C₁₀, C₁₂, C₁₄, C₁₆, C₁₈, C_{20 aldehydes) into fatty alcohols. Specific examples of functional equivalents include long-chain alcohol dehydrogenases, having the ability to utilize long-chain aldehydes (e.g., C}₁₆, C₁₈, C₂₀) as substrates. In particular embodiments, the alcohol dehydrogenase has the amino acid sequence of SEQ ID NO:105 (encoded by the polynucleotide sequence of SEQ ID NO:104), or an active fragment or variant of this sequence. In some embodiments, the alcohol dehydrogenase has the amino acid sequence of SEQ ID NO:107 (encoded by the polynucleotide sequence of SEQ ID NO:106), or an active fragment or variant of this sequence.

Certain aspects may include modified photosynthetic microorganisms having reduced expression of an endogenous alcohol dehydrogenase. For instance, where the production of fatty acids, triglycerides, or alkanes/alkenes is desired, reduced expression of an alcohol dehydrogenase can shunt carbon away from production of fatty alcohols and towards fatty acids, triglycerides, or alkanes/alkenes, thereby increasing production of these latter lipids. Thus, certain aspects include mutations (e.g., genomic) such as point mutations or insertions that reduce or eliminate the expression and/or enzymatic activity of one or more endogenous alcohol dehydrogenases. Also included are full or partial deletions of an endogenous gene encoding an alcohol dehydrogenase involved in fatty alcohol synthesis.

Aldehyde Decarbonylases.

Embodiments of the present invention optionally include one or more introduced or expressed (e.g., overexpressed) alcohol decarbonylases. As used herein, an “aldehyde decarbonylase” is capable of catalyzing the conversion of an acyl aldehyde (or fatty aldehyde) to an alkane or alkene. Introduction or overexpression of an aldehyde decarbonylase can thus increase the production of alkanes and/or alkenes, optionally in combination with introduction or overexpression of an acyl-ACP reductase, and optionally in further combination with reduced expression of an aldehyde dehydrogenase, to shunt carbon away from fatty acids and towards alkanes/alkenes.

Included are members of the ferritin-like or ribonucleotide reductase-like family of nonheme diiron enzymes (see, e.g., Stubbe et al., Trends Biochem Sci. 23:438-43, 1998). Examples include PCC7942_orf1593 and PCC6803_orfsll0208 from Synechostis sp. PCC6803 and orthologs thereof, which can be found, for example, in N. punctiforme PCC73102, Thermosynechococcus elongatus BP-1, Synechococcus sp. Ja-3-3AB, P. marinus MIT9313, P. marinus NATL2A, and Synechococcus sp. RS 9117, the latter having at least two paralogs (RS 9117-1 and -2).

Certain embodiments include photosynthetic microorganism having reduced expression of one or more endogenous aldehyde decarbonylases. According to one non-limiting theory, because the aldehyde decarbonylase encoded by PCC7942_orf1593 or PCC6803_orfsll0208 (from Synechostis sp. PCC6803) utilizes acyl aldehyde as a substrate for alkane or alkene production, reducing expression of this protein may further increase yields of free fatty acids by shunting acyl aldehydes (produced by acyl-ACP reductase) away from an alkane-producing pathway, and towards a fatty acid-producing pathway. Included are mutations (e.g., genomic) that reduce or eliminate the expression and/or enzymatic activity of one or more endogenous aldehyde decarbonylases. Also included are full or partial deletions of an endogenous gene encoding an aldehyde decarbonylase.

Aldehyde Dehydrogenases.

Embodiments of the present invention optionally include one or more aldehyde dehydrogenases. Examples of aldehyde dehydrogenases include enzymes capable of using acyl aldehydes (e.g., nonyl-aldehyde, C16 fatty aldehyde) as a substrate, and converting them into fatty acids. In certain embodiments, the aldehyde dehydrogenase is naturally-occurring or endogenous to the modified microorganism, and is sufficient to convert increased acyl aldehydes (produced by acyl-ACP reductase) into fatty acids, and thereby contribute to increased fatty acid production and overall satisfactory growth characteristics.

In certain embodiments, the aldehyde dehydrogenase can be overexpressed, for example, by recombinantly introducing a polynucleotide that encodes the enzyme, increasing expression of an endogenous enzyme, or both. An aldehyde dehydrogenase can be overexpressed in a strain that already expresses a naturally-occurring or endogenous enzyme, to further increase fatty acid production of an acyl-ACP reductase over-expressing strain and/or improve its growth characteristics, relative, for example, to an acyl-ACP reductase-overexpressing strain that only expresses endogenous aldehyde dehydrogenase. An aldehyde dehydrogenase can also be expressed or overexpressed in a strain that does not have a naturally occurring aldehyde dehydrogenase of that type, e.g., it does not naturally express an enzyme that is capable of efficiently converting acyl aldehydes such as nonyl-aldehyde into fatty acids.

In these and related embodiments, expression or overexpression of an aldehyde dehydrogenase may increase shunting of acyl aldehydes towards production of fatty acids, and away from production of other products such as fatty alcohols alkanes. It may also reduce accumulation of potentially toxic acyl aldehydes, and thereby improve growth characteristics of a modified microorganism.

One exemplary aldehyde dehydrogenase is encoded by orf0489 of Synechococcus elongatus PCC7942. Also included are homologs or paralogs thereof, functional equivalents thereof, and fragments or variants thereofs. Functional equivalents can include aldehyde dehydrogenases with the ability to efficiently convert acyl aldehydes (e.g., nonyl-aldehyde) into fatty acids. In specific embodiments, the aldehyde dehydrogenase has the amino acid sequence of SEQ ID NO:103 (encoded by the polynucleotide sequence of SEQ ID NO:102), or an active fragment or variant of this sequence.

Certain embodiments include photosynthetic microorganism having reduced expression and/or activity of one or more endogenous aldehyde dehydrogenases, particularly those associated with production of fatty acids. In these and related embodiment, reducing the activity of endogenous aldehyde dehydrogenase can shunt carbon away from fatty acids and towards other desired carbon-containing compounds, such as alkanes/alkenes, fatty alcohols, and/or wax esters. Included are mutations (e.g., genomic) that reduce or eliminate the expression and/or enzymatic activity of one or more endogenous aldehyde dehydrogenases. Also included are full or partial deletions of an endogenous gene encoding an aldehyde dehydrogenase, such as orf0489 from Synechococcus elongatus PCC7942.

### Glucose Secretion Proteins

In additional embodiments, the modified photosynthetic microorganism with reduced glycogen accumulation are further modified to include one or more introduced or overexpressed polynucleotides involved in glucose secretion, to allow for continued secretion of glucose from glycogen deficient strains that are placed under stress conditions. Examples of such polypeptides include glucose permeases and glucose/H+ symporters, such as glcP (e.g., Bacillus subtilis168 glcP; NCBI NP_{—}388933; SEQ ID NO:176), glcP1 (e.g., Streptomyces coelicolor glcP1; NCBI NP_{—}629713.1; SEQ ID NO:177), glcP2 (e.g., Streptomyces coelicolor A3 glcP2; NCBI NP_{—}631212; SEQ ID NO:178), and Mycobacterium smegmatis MC2 155 (NCBI YP_{—}888461; SEQ ID NO:179), and functional fragments and variants thereof.

### Isobutanol/Isopentanol Synthesis Proteins

In particular embodiments, the modified photosynthetic microorganism with reduced glycogen accumulation are further modified to express one or more polypeptides associated with isobutanol and/or isopentanol biosynthesis, to allow for continued production of isobutanol and/or isopentanol from glycogen deficient strains that are placed under stress conditions. Examples of such polypeptides include 2-keto acid decarboxylases and certain alcohol dehydrogenases. For instance, one exemplary polypeptide includes an alpha-ketoisovalerate decarboxylase (2-keto acid decarboxylase) from Lactococcus lactis (SEQ ID NO:181), and another exemplary polypeptide includes an alcohol dehydrogenase (YqhD) from E. coli (SEQ ID NO:183). Also included are active variants and fragments thereof.

### 4-Hydroxybutyrate/1,4-Butanediol Synthesis Proteins

In some embodiments, the modified photosynthetic microorganism with reduced glycogen accumulation are further modified to express one or more polypeptides associated with 4-hydroxybutyrate and optionally 1,4-butanediol biosynthesis, to allow for continued production of 4-hydroxybutyrate and optionally 1,4-butanediol from glycogen deficient strains that are placed under stress conditions. Examples of such polypeptides include alpha-ketoglutarate decarboxylases, 4-hydroxybutyrate dehydrogenases, succinyl-CoA synthetases, succinate-semialdehyde dehydrogenases, 4-hydroxybutyryl-CoA transferases, and aldehyde/alcohol dehydrogenases (see FIG. 22). Specific examples include alpha-ketoglutarate decarboxylases encoded by CCDC5180_{—}0513 (SEQ ID NO:200) from Mycobacterium bovis and SYNPCC7002_A2770 (SEQ ID NO:202) from Synechococcus sp PCC 7002; 4-hydroxybutyrate dehydrogenases encoded by PGN_{—}0724 (SEQ ID NO:204) from Porphyromonas gingivalis and CKR_{—}2662 (SEQ ID NO:206) from Clostridium kluyveri; succinyl-CoA synthetases encoded by the alpha subunit sucC (b0728) (SEQ ID NO:214) from E. coli and the beta subunit sucD (b0729) (SEQ ID NO:216) from E. coli; succinate-semialdehyde dehydrogenase encoded by PGTDC60_{—}1813 (SEQ ID NO:218) from Porphyromonas gingivalis; 4-hydroxybutyryl-CoA transferases encoded by cat2 (CKR_{—}2666) (SEQ ID NO:208) from Clostridium kluyveri, and homologs from Clostridium aminobutyricum and Porphyromonas gingivalis; and aldehyde/alcohol dehydrogenases encoded by adhE2 (CEA_P0034) (SEQ ID NO:210) from Clostridium acetobutylicum and adhE (b1241) (SEQ ID NO:212) from E. coli. Also included are active variants and fragments thereof.

### Polyamine Synthesis Proteins

In certain embodiments, the modified photosynthetic microorganism with reduced glycogen accumulation are further modified to express one or more polypeptides associated with polyamine biosynthesis, to allow for continued production of polyamines or intermediates thereof from glycogen deficient strains that are placed under stress conditions. Exemplary polyamine precursors include agmatine and putrescine. Examples of such polypeptides include arginine decarboxylases to convert L-arginine into agmatine, agmatine deiminases to convert agmatine into N-carbamoylputrescine, and N-carbamoylputrescine amidases to convert N-carbamoylputrescine into putrescine. One example of an arginine decarboxylase is encoded by Synpcc7942_{—}1037 (SEQ ID NO:220) from S. elongatus PCC7942. Specific examples of agmatine deiminases are encoded by Synpcc7942_{—}2402 (SEQ ID NO:222) and Synpcc7942_{—}2461 from S. elongatus PCC7942. One exemplary N-carbamoylputrescine amidase is encoded by Synpcc7942_{—}2145 (SEQ ID NO:224) from S. elongatus PCC7942. Also included are active variants and fragments thereof.

### Polypeptide Variants and Fragments

As noted above, embodiments of the present invention include variants and fragments of any of the reference polypeptides and polynucleotides described herein (see, e.g., the Sequence Listing). Variant polypeptides are biologically active, that is, they continue to possess the enzymatic activity of a reference polypeptide. Such variants may result from, for example, genetic polymorphism and/or from human manipulation.

Biologically active variants of a reference polypeptide will have at least 40%, 50%, 60%, 70%, generally at least 75%, 80%, 85%, usually about 90% to 95% or more, and typically about 97% or 98% or more sequence similarity or identity to the amino acid sequence for a reference protein as determined by sequence alignment programs described elsewhere herein using default parameters. A biologically active variant of a reference polypeptide may differ from that protein generally by as much 200, 100, 50 or 20 amino acid residues or suitably by as few as 1-15 amino acid residues, as few as 1-10, such as 6-10, as few as 5, as few as 4, 3, 2, or even 1 amino acid residue. In some embodiments, a variant polypeptide differs from the reference sequences referred to herein (see, e.g., the Sequence Listing) by at least one but by less than 15, 10 or 5 amino acid residues. In other embodiments, it differs from the reference sequences by at least one residue but less than 20%, 15%, 10% or 5% of the residues.

A biologically active fragment can be a polypeptide fragment which is, for example, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 220, 240, 260, 280, 300, 320, 340, 360, 380, 400, 450, 500, 600 or more contiguous amino acids, including all integers in between, of a reference polypeptide sequence.

A reference polypeptide may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (“Molecular Biology of the Gene”, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of reference polypeptides. Recursive ensemble mutagenesis (REM), a technique which enhances the frequency of functional mutants in the libraries, can be used in combination with the screening assays to identify polypeptide variants (Arkin and Yourvan (1992) Proc. Natl. Acad. Sci. USA 89: 7811-7815; Delgrave et al., (1993) Protein Engineering, 6: 327-331). Conservative substitutions, such as exchanging one amino acid with another having similar properties, may be desirable as discussed in more detail below.

Polypeptide variants may contain conservative amino acid substitutions at various locations along their sequence, as compared to a reference amino acid sequence. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al., (1978), A model of evolutionary change in proteins. Matrices for determining distance relationships In M. O. Dayhoff, (ed.), Atlas of protein sequence and structure, Vol. 5, pp. 345-358, National Biomedical Research Foundation, Washington DC; and by Gonnet et al., (Science, 256: 14430-1445, 1992), however, include proline in the same group as glycine, serine, alanine and threonine. Accordingly, for the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behaviour.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always non-aromatic. Dependent on their structural properties, amino acid residues may fall in two or more classes. For the naturally-occurring protein amino acids, sub-classification according to this scheme is presented in Table A.

TABLE A
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine, |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional truncated and/or variant polypeptide can readily be determined by assaying its enzymatic activity, as described herein. Conservative substitutions are shown in Table B under the heading of exemplary substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE B
Exemplary Amino Acid Substitutions

| Original | Exemplary | Preferred |
|---|---|---|
| Residue | Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm. C. Brown Publishers (1993).

Thus, a predicted non-essential amino acid residue in reference polypeptide is typically replaced with another amino acid residue from the same side chain family. Alternatively, mutations can be introduced randomly along all or part of a coding sequence, such as by saturation mutagenesis, and the resultant mutants can be screened for an activity of the parent polypeptide to identify mutants which retain that activity. Following mutagenesis of the coding sequences, the encoded peptide can be expressed recombinantly and the activity of the peptide can be determined. A “non-essential” amino acid residue is a residue that can be altered from the wild-type sequence of an embodiment polypeptide without abolishing or substantially altering one or more of its activities. Suitably, the alteration does not substantially abolish one of these activities, for example, the activity is at least 20%, 40%, 60%, 70% or 80% 100%, 500%, 1000% or more of wild-type. An “essential” amino acid residue is a residue that, when altered from the wild-type sequence of a reference polypeptide, results in abolition of an activity of the parent molecule such that less than 20% of the wild-type activity is present. For example, such essential amino acid residues may include those that are conserved in the enzymatic sites of reference polypeptides from various sources.

Accordingly, the present invention also contemplates variants of the naturally-occurring reference polypeptide sequences or their biologically-active fragments, wherein the variants are distinguished from the naturally-occurring sequence by the addition, deletion, or substitution of one or more amino acid residues. In general, variants will display at least about 30, 40, 50, 55, 60, 65, 70, 75, 80, 85, 90, 91, 92, 93, 94, 95, 96, 97, 98, 99% similarity or sequence identity to a reference polypeptide sequence. Moreover, sequences differing from the native or parent sequences by the addition, deletion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100 or more amino acids but which retain the properties of a parent or reference polypeptide sequence are contemplated.

In some embodiments, variant polypeptides differ from a reference polypeptide sequence by at least one but by less than 50, 40, 30, 20, 15, 10, 8, 6, 5, 4, 3 or 2 amino acid residue(s). In other embodiments, variant polypeptides differ from a reference sequence by at least 1% but less than 20%, 15%, 10% or 5% of the residues. (If this comparison requires alignment, the sequences should be aligned for maximum similarity. “Looped” out sequences from deletions or insertions, or mismatches, are considered differences.)

In certain embodiments, a variant polypeptide includes an amino acid sequence having at least about 50%, 55%, 60%, 65%, 70%, 75%, 80%, 85%, 90%, 91%, 92%, 93%, 94% 95%, 96%, 97%, 98% or more sequence identity or similarity to a corresponding sequence of a reference polypeptide described herein, and retains the enzymatic activity of that reference polypeptide.

Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch, (1970, J. Mol. Biol. 48: 444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997, Nucleic Acids Res, 25: 3389-3402). When utilizing BLAST and Gapped BLAST programs, the default parameters of the respective programs (e.g., XBLAST and NBLAST) can be used.

In one embodiment, as noted above, polynucleotides and/or polypeptides can be evaluated using a BLAST alignment tool. A local alignment consists simply of a pair of sequence segments, one from each of the sequences being compared. A modification of Smith-Waterman or Sellers algorithms will find all segment pairs whose scores cannot be improved by extension or trimming, called high-scoring segment pairs (HSPs). The results of the BLAST alignments include statistical measures to indicate the likelihood that the BLAST score can be expected from chance alone.

The raw score, S, is calculated from the number of gaps and substitutions associated with each aligned sequence wherein higher similarity scores indicate a more significant alignment. Substitution scores are given by a look-up table (see PAM, BLOSUM).

Gap scores are typically calculated as the sum of G, the gap opening penalty and L, the gap extension penalty. For a gap of length n, the gap cost would be G+Ln. The choice of gap costs, G and L is empirical, but it is customary to choose a high value for G (10-15), e.g., 11, and a low value for L (1-2) e.g., 1.

The bit score, S′, is derived from the raw alignment score S in which the statistical properties of the scoring system used have been taken into account. Bit scores are normalized with respect to the scoring system, therefore they can be used to compare alignment scores from different searches. The terms “bit score” and “similarity score” are used interchangeably. The bit score gives an indication of how good the alignment is; the higher the score, the better the alignment.

The E-Value, or expected value, describes the likelihood that a sequence with a similar score will occur in the database by chance. It is a prediction of the number of different alignments with scores equivalent to or better than S that are expected to occur in a database search by chance. The smaller the E-Value, the more significant the alignment. For example, an alignment having an E value of e⁻¹¹⁷ means that a sequence with a similar score is very unlikely to occur simply by chance. Additionally, the expected score for aligning a random pair of amino acids is required to be negative, otherwise long alignments would tend to have high score independently of whether the segments aligned were related. Additionally, the BLAST algorithm uses an appropriate substitution matrix, nucleotide or amino acid and for gapped alignments uses gap creation and extension penalties. For example, BLAST alignment and comparison of polypeptide sequences are typically done using the BLOSUM62 matrix, a gap existence penalty of 11 and a gap extension penalty of 1.

In one embodiment, sequence similarity scores are reported from BLAST analyses done using the BLOSUM62 matrix, a gap existence penalty of 11 and a gap extension penalty of 1.

In a particular embodiment, sequence identity/similarity scores provided herein refer to the value obtained using GAP Version 10 (GCG, Accelrys, San Diego, Calif.) using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix (Henikoff and Henikoff, PNAS USA. 89:10915-10919, 1992). GAP uses the algorithm of Needleman and Wunsch (J Mol Biol. 48:443-453, 1970) to find the alignment of two complete sequences that maximizes the number of matches and minimizes the number of gaps.

In one particular embodiment, the variant polypeptide comprises an amino acid sequence that can be optimally aligned with a reference polypeptide sequence (see, e.g., Sequence Listing) to generate a BLAST bit scores or sequence similarity scores of at least about 50, 60, 70, 80, 90, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or more, including all integers and ranges in between, wherein the BLAST alignment used the BLOSUM62 matrix, a gap existence penalty of 11, and a gap extension penalty of 1.

Variants can be identified by screening combinatorial libraries of mutants of a reference polypeptide. Libraries or fragments e.g., N terminal, C terminal, or internal fragments, of protein coding sequence can be used to generate a variegated population of fragments for screening and subsequent selection of variants of a reference polypeptide.

Methods for screening gene products of combinatorial libraries made by point mutation or truncation, and for screening cDNA libraries for gene products having a selected property are known in the art. Such methods are adaptable for rapid screening of the gene libraries generated by combinatorial mutagenesis of polypeptides.

The present invention also contemplates the use of chimeric or fusion proteins for increasing lipid or fatty acid production and/or producing triglycerides. As used herein, a “chimeric protein” or “fusion protein” includes a reference polypeptide or a polypeptide fragment thereof linked to either another reference polypeptide (e.g., to create multiple fragments), to a non-reference polypeptide, or to both. A “non-reference polypeptide” refers to a “heterologous polypeptide” having an amino acid sequence corresponding to a protein which is different from the reference protein sequence, and which is derived from the same or a different organism. The reference polypeptide of the fusion protein can correspond to all or a portion of a biologically active amino acid sequence. In certain embodiments, a fusion protein includes at least one or two biologically active portions of a reference polypeptide. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order.

The fusion partner may be designed and included for essentially any desired purpose provided they do not adversely affect the enzymatic activity of the polypeptide. For example, in one embodiment, a fusion partner may comprise a sequence that assists in expressing the protein (an expression enhancer) at higher yields than the native recombinant protein. Other fusion partners may be selected so as to increase the solubility or stability of the protein or to enable the protein to be targeted to desired intracellular compartments.

The fusion protein can include a moiety which has a high affinity for a ligand. For example, the fusion protein can be a GST-fusion protein in which the reference polypeptide sequences are fused to the C-terminus of the GST sequences. Such fusion proteins can facilitate the purification and/or identification of the resulting polypeptide. Alternatively, the fusion protein can be reference polypeptide containing a heterologous signal sequence at its N-terminus. In certain host cells, expression and/or secretion of such proteins can be increased through use of a heterologous signal sequence.

Fusion proteins may generally be prepared using standard techniques. For example, DNA sequences encoding the polypeptide components of a desired fusion may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures, if desired. Such a peptide linker sequence is incorporated into the fusion protein using standard techniques well known in the art. Certain peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39 46 (1985); Murphy et al., Proc. Natl. Acad. Sci. USA 83:8258 8262 (1986); U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences may be operably linked to suitable transcriptional or translational regulatory elements. Certain of the regulatory elements responsible for expression of DNA are located 5′ to the DNA sequence encoding the first polypeptides. Similarly, other regulatory elements such as stop codons required to end translation and transcription termination signals are present 3′ to the DNA sequence encoding the second polypeptide.

In general, polypeptides and fusion polypeptides (as well as their encoding polynucleotides) are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

### Polynucleotides and Vectors

Certain modified photosynthetic microorganisms (e.g., Cyanobacteria) of the present invention comprise one or more introduced polynucleotides encoding a reference polypeptide described herein. Examples include polynucleotides that encode one or more polypeptides associated with glycogen breakdown, secretion of glycogen precursors, glucose secretion, and biosynthesis of lipids or other carbon-containing compounds described herein, such as isobutanol, isopentanol, 4-hydroxybutyrate, 1,4-butanediol, and polyamines or intermediates thereof such as agmatine and putrescine. Also included are nucleotide sequences that encode any functional naturally-occurring variants or fragments (i.e., allelic variants, orthologs, splice variants) or non-naturally occurring variants or fragments of these native enzymes (i.e., optimized by engineering), as well as compositions comprising such polynucleotides, including, e.g., cloning and expression vectors.

As used herein, the terms “DNA” and “polynucleotide” and “nucleic acid” refer to a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

As will be understood by those skilled in the art, the polynucleotide sequences of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will be recognized by the skilled artisan, polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

Polynucleotides may comprise a native sequence (e.g., an endogenous sequence that encodes an acyl-ACP reductase, an ACP, a diacylglycerol acyltransferase, a fatty acyl-CoA synthetase, a glycogen breakdown protein, an acetyl-CoA carboxylase, aldehyde dehydrogenase, or a portion thereof) or may comprise a variant, or a biological functional equivalent of such a sequence.

Polynucleotide variants may contain one or more substitutions, additions, deletions and/or insertions, as further described below, preferably such that the enzymatic activity of the encoded polypeptide is not substantially diminished relative to the unmodified polypeptide. The effect on the enzymatic activity of the encoded polypeptide may generally be assessed as described herein.

In certain embodiments, a modified photosynthetic microorganism comprises one or more polynucleotides encoding one or more acyl-ACP reductase polypeptides. Exemplary acyl-ACP reductase nucleotide sequences include orf1954 from Synechococcus elongatus PCC7942 (SEQ ID NO:1), and orfsll0209 from Synechocystis sp. PCC6803 (SEQ ID NO:3).

In certain embodiments, a modified photosynthetic microorganism comprises one or more polynucleotides encoding one or more acyl carrier proteins (ACP). Exemplary ACP nucleotide sequences include SEQ ID NO:5 from Synechococcus elongatus PCC7942, SEQ ID NOS:7, 9, and 11 from Acinetobacter sp. ADP1, and SEQ ID NO:13 from Spinacia oleracea.

In certain embodiments of the present invention, a polynucleotide encodes an acetyl-CoA carboxylase (ACCase) comprising or consisting of a polypeptide sequence set forth in any of SEQ ID NOs:55, 45, 46, 47, 48 or 49, or a fragment or variant thereof. In particular embodiments, a ACCase polynucleotide comprises or consists of a polynucleotide sequence set forth in any of SEQ ID NOs:56, 57, 50, 51, 52, 53 or 54, or a fragment or variant thereof. SEQ ID NO:55 is the sequence of Saccharomyces cerevisiae acetyl-CoA carboxylase (yAcc1); and SEQ ID NO:56 is a codon-optimized for expression in Cyanobacteria sequence that encodes yAcc1. SEQ ID NO:45 is Synechococcus sp. PCC 7002 AccA; SEQ ID NO:46 is Synechococcus sp. PCC 7002 AccB; SEQ ID NO:47 is Synechococcus sp. PCC 7002 AccC; and SEQ ID NO:48 is Synechococcus sp. PCC 7002 AccD. SEQ ID NO:50 encodes Synechococcus sp. PCC 7002 AccA; SEQ ID NO:51 encodes Synechococcus sp. PCC 7002 AccB; SEQ ID NO:52 encodes Synechococcus sp. PCC 7002 AccC; and SEQ ID NO:53 encodes Synechococcus sp. PCC 7002 AccD. SEQ ID NO:49 is a Triticum aestivum ACCase; and SEQ ID NO:54 encodes this Triticum aestivum ACCase.

In certain embodiments, a modified photosynthetic microorganism comprises one or more polynucleotides encoding one or more DGAT enzymes. In certain embodiments of the present invention, a polynucleotide encodes a DGAT comprising of consisting of a polypeptide sequence set forth in any one of SEQ ID NOs:58, 59, 60 or 61, or a fragment or variant thereof. SEQ ID NO:58 is the sequence of DGATn; SEQ ID NO: 59 is the sequence of Streptomyces coelicolor DGAT (ScoDGAT or SDGAT); SEQ ID NO:60 is the sequence of Alcanivorax borkumensis DGAT (AboDGAT); and SEQ ID NO:61 is the sequence of DGATd (Acinetobacter baylii sp.). In certain embodiments of the present invention, a DGAT polynucleotide comprises or consists of a polynucleotide sequence set forth in any one of SEQ ID NOs:62, 63, 64, 65 or 66, or a fragment or variant thereof. SEQ ID NO:62 is a codon-optimized for expression in Cyanbacteria sequence that encodes DGATn; SEQ ID NO: 63 has homology to SEQ ID NO:62; SEQ ID NO:64 is a codon-optimized for expression in Cyanobacteria sequence that encodes ScoDGAT; SEQ ID NO:65 is a codon-optimized for expression in Cyanobacteria sequence that encodes AboDGAT; and SEQ ID NO:66 is a codon-optimized for expression in Cyanobacteria sequence that encodes DGATd. DGATn and DGATd correspond to Acinetobacter baylii DGAT and a modified form thereof, which includes two additional amino acid residues immediately following the initiator methionine.

Certain embodiments employ one or more fatty acyl-CoA synthetase encoding polynucleotide sequences. One exemplary fatty acyl-CoA synthetase includes the FadD gene from E. coli (SEQ ID NO:16) which encodes a fatty acyl-CoA synthetase having substrate specificity for medium and long chain fatty acids. Other exemplary fatty acyl-CoA synthetases include those derived from S. cerevisiae; for example, the Faa1p coding sequence is set forth in SEQ ID NO:18, the Faa2p coding sequence is set forth in SEQ ID NO:20, and the Faa3p is set forth in SEQ ID NO:22. SEQ ID NO:22 is codon-optimized for expression is S. elongatus PCC7942.

Certain embodiments may employ one or more aldehyde dehydrogenase encoding polynucleotide sequences. One exemplary aldehyde dehydrogenase is orf0489 of Synechococcus elongatus PCC7942 (SEQ ID NO:102). Also included are active fragments or variants of this sequence.

Certain embodiments may employ one or more alcohol dehydrogenase encoding polynucleotide sequences. Exemplary alcohol dehydrogenases include slr1192 of Synechocystis sp. PCC6803 (SEQ ID NO:104) and ACIAD3612 from Acinetobacter baylyi (SEQ ID NO:106).

In certain embodiments of the present invention, a modified photosynthetic microorganism comprise one or more polynucleotides encoding one or more polypeptides associated with a glycogen breakdown, or a fragment or variant thereof. In particular embodiments, the one or more polypeptides are glycogen phosphorylase (GlgP), glycogen isoamylase (GlgX), glucanotransferase (MalQ), phosphoglucomutase (Pgm), glucokinase (Glk), and/or phosphoglucose isomerase (Pgi), or a functional fragment or variant thereof. A representative glgP polynucleotide sequence is provided in SEQ ID NO:67, and a representative GlgP polypeptide sequence is provided in SEQ ID NO:68. A representative glgX polynucleotide sequence is provided in SEQ ID NO:69, and a representative GlgX polypeptide sequence is provided in SEQ ID NO:70. A representative malQ polynucleotide sequence is provided in SEQ ID NO:71, and a representative MalQ polypeptide sequence is provide in SEQ ID NO:72. A representative phosphoglucomutase (pgm) polynucleotide sequence is provided in SEQ ID NO:24, and a representative phosphoglucomutase (Pgm) polypeptide sequence is provided in SEQ ID NO:73, with others provided infra (SEQ ID NOs:25, 26, 74-81). A representative glk polynucleotide sequence is provided in SEQ ID NO:82, and a representative Glk polypeptide sequence is provided in SEQ ID NO:83. A representative pgi polynucleotide sequence is provided in SEQ ID NO:84, and a representative Pgi polypeptide sequence is provided in SEQ ID NO:85. In particular embodiments of the present invention, a polynucleotide comprises one of these polynucleotide sequences, or a fragment or variant thereof, or encodes one of these polypeptide sequences, or a fragment or variant thereof.

In certain embodiments, the present invention provides isolated polynucleotides comprising various lengths of contiguous stretches of sequence identical to or complementary to a polypeptide described herein, such as an acyl-ACP reductase, acyl carrier protein (ACP), acetyl-CoA carboxylase (ACCase), glycogen breakdown protein, diacylglycerol acyltransferase (DGAT), aldehyde dehydrogenase, or fatty acyl-CoA synthetase, wherein the isolated polynucleotides encode a biologically active, truncated enzyme.

In certain embodiments, a modified photosynthetic microorganism comprises one or more polynucleotides encoding one or more thioesterases (TES) including acyl-ACP thioesterases and/or acyl-CoA thioesterases. In certain embodiments, the polynucleotide sequence of the TES encodes a TesA or TesB polypeptide from E. coli, or a cytoplasmic TesA variant (*TesA) having the sequence set forth in SEQ ID NO:121.

In certain embodiments, the polynucleotide sequence of the TES comprises that of the FatB gene, encoding a FatB enzyme, such as a C8, C12, C14, C16, or C18 FatB enzyme. In certain embodiments, the polynucleotide encodes a thioesterase (e.g., FatB thioesterase), having only thioesterase activity and little or no lysophospholipase activity. In specific embodiments, the thioesterase is a FatB acyl-ACP thioesterase, which can hydrolyze acyl-ACP but not acyl-CoA. SEQ ID NO:197 is an exemplary nucleotide sequence of a C8/C10 FatB2 thioesterase derived from Cuphea hookeriana, and SEQ ID NO:122 is codon-optimized for expression in Cyanobacteria. SEQ ID NO:123 is an exemplary nucleotide sequence of a C12 FatB1 acyl-ACP thioesterase derived from Umbellularia californica, and SEQ ID NO:124 is a codon-optimized version of SEQ ID NO:123 for optimal expression in Cyanobacteria. SEQ ID NO:126 is an exemplary nucleotide sequence of a C14 FatB1 thioesterase derived from Cinnamomum camphora, and SEQ:125 is a codon-optimized version of SEQ ID NO:126. SEQ ID NO:127 is an exemplary nucleotide sequence of a C16 FatB1 thioesterase derived from Cuphea hookeriana, and SEQ ID NO:128 is a codon-optimized version of SEQ ID NO:127. In certain embodiments, one or more FatB sequences are operably linked to a strong promoter, such as a Ptrc promoter. In other embodiments, one or more FatB sequences are operably linked to a relatively weak promoter, such as an arabinose promoter.

In certain embodiments of the present invention, a polynucleotide encodes a phosphatidate phosphatase (also referred to as a phosphatidic acid phosphatase; PAP) comprising or consisting of a polypeptide sequence set forth in SEQ ID NO:131, or a fragment or variant thereof. In particular embodiments, a phosphatidate phosphatase polynucleotide comprises or consists of a polynucleotide sequence set forth in SEQ ID NO:129 or SEQ ID NO:130, or a fragment or variant thereof. SEQ ID NO:131 is the sequence of Saccharomyces cerevisiae phosphatidate phosphatase (yPAH1), and SEQ ID NO:129 is a codon-optimized for expression in Cyanobacteria sequence that encodes yPAH1. In certain embodiments, the nucleotide sequence of the PAP is derived from the E. coli PgpB gene, and/or the PAP gene from Synechocystis sp. PCC6803.

In certain embodiments of the present invention, a modified photosynthetic microorganism comprises one or more polynucleotides encoding one or more phospholipases, including lysophospholipases, or a fragment or variant thereof. In certain embodiments, the encoded lysophospholipase is Lysophospholipase L1 (TesA), Lysophospholipase L2, TesB, Vu Patatin 1 protein, or a homolog thereof.

In particular embodiments, the encoded phospholipase, e.g., a lysophospholipase, is a bacterial phospholipase, or a fragment or variant thereof, and the polynucleotide comprises a bacterial phospholipase polynucleotide sequence, e.g., a sequence derived from Escherichia coli, Enterococcus faecalis, or Lactobacillus plantarum. In particular embodiments, the encoded phospholipase is Lysophospholipase L1 (TesA), Lysophospholipase L2, TesB, Vu Patatin 1 protein, or a functional fragment thereof.

In certain embodiments, a lysophospholipase is a bacterial Lysophospholipase L1 (TesA) or TesB, such as an E. coli Lysophospholipase L1 encoded by a polynucleotide (pldC) having the wild-type sequence set forth in SEQ ID NO:196, or an E. coli TesB encoded by a polynucleotide having the wild-type sequence set forth in SEQ ID NO:132. The polypeptide sequence of E. coli Lysophospholipase L1 is provided in SEQ ID NO:133, and the polypeptide sequence of E. coli TesB is provided in SEQ ID NO:134. In other embodiments, a lysophospholipase is a Lysophospholipase L2, such as an E. coli Lysophospholipase L2 encoded by a polynucleotide (pldB) having the wild-type sequence set forth in SEQ ID NO:135, or a Vu patatin 1 protein encoded by a polynucleotide having the wild-type sequence set forth in SEQ ID NO:136. The polypeptide sequence of E. coli Lysophospholipase L2 is provided in SEQ ID NO:137, and the polypeptide sequence of Vu patatin 1 protein is provided in SEQ ID NO:138.

In particular embodiments, the polynucleotide encoding the phospholipase variant is modified such that it encodes a phospholipase that localizes predominantly to the cytoplasm instead of the periplasm. For example, it may encode a phospholipase having a deletion or mutation in a region associated with periplasmic localization. In particular embodiments, the encoded phospholipase variant is derived from Lysophospholipase L1 (TesA). In certain embodiments, the Lysophospholipase L1 (TesA) variant is a bacterial TesA, such as an E. coli Lysophospholipase (TesA) variant encoded by a polynucleotide having the sequence set forth in SEQ ID NO:139. The polypeptide sequence of the Lysophospholipase L1 variant is provided in SEQ ID NO:121 (PldC(*TesA)).

Additional examples of phospholipase-encoding polynucleotide sequences include phospholipase A1 (PldA) from Acinetobacter sp. ADP1 (SEQ ID NO:140), phospholipase A (PldA) from E. coli (SEQ ID NO:141), phospholipase from Streptomyces coelicolor A3(2) (SEQ ID NO:142), phospholipase A2 (PLA2-α) from Arabidopsis thaliana (SEQ ID NO:143). phospholipase A1/triacylglycerol lipase (DAD1; Defective Anther Dehiscence 1) from Arabidopsis thaliana (SEQ ID NO:144), chloroplast DONGLE from Arabidopsis thaliana (SEQ ID NO:145), patatin-like protein from Arabidopsis thaliana (SEQ ID NO:146), and patatin from Anabaena variabilis ATCC 29413 (SEQ ID NO:147). Additional non-limiting examples of lysophospholipase-encoding polynucleotide sequences include phospholipase B (Plb1p) from Saccharomyces cerevisiae S288c (SEQ ID NO:148), phospholipase B (Plb2p) from Saccharomyces cerevisiae S288c (SEQ ID NO:149), ACIAD1057 (TesA homolog) from Acinetobacter ADP1 (SEQ ID NO:150), ACIAD1943 lysophospholipase from Acinetobacter ADP1 (SEQ ID NO:151), and a lysophospholipase (YP_{—}702320; RHA1_ro02357) from Rhodococcus (SEQ ID NO:152).

Certain embodiments employ one or more TAG hydrolase encoding polynucleotide sequences. Non-limiting examples of TAG hydrolase polynucleotide sequences include SDP1 (SUGAR-DEPENDENT1) triacylglycerol lipase from Arabidopsis thaliana (SEQ ID NO:153), ACIAD1335 from Acinetobacter sp. ADP1 (SEQ ID NO:154), TG14P from S. cerevisiae (SEQ ID NO:175), and RHA1_ro04722 (YP_{—}704665) TAG lipase from Rhodococcus (SEQ ID NO:155). Additional polynucleotide sequences for exemplary lipases/esterases include RHA1_ro01602 lipase/esterase from Rhodococcus sp. (see SEQ ID NO:156), and the RHA1_ro06856 lipase/esterase (see SEQ ID NO:119) from Rhodococcus sp.

Particular embodiments employ glucose permease or glucose/H+ symporter encoding polynucleotide sequences. Non-limiting examples include those that encode glcP (e.g., Bacillus subtilis168 glcP; NCBI NP_{—}388933; SEQ ID NO:176), glcP1 (e.g., Streptomyces coelicolor glcP1; NCBI NP_{—}629713.1; SEQ ID NO:177), glcP2 (e.g., Streptomyces coelicolor A3 glcP2; NCBI NP_{—}631212; SEQ ID NO:178), and Mycobacterium smegmatis MC2 155 (NCBI YP_{—}888461; SEQ ID NO:179).

Certain embodiments employ polynucleotides that encode polypeptides associated with the biosynthesis of isobutanol and/or isopentanol. Examples of such polynucleotides include those that encode a 2-keto acid decarboxylase and those that encode an alcohol dehydrogenase. Specific examples include the alpha-ketoisovalerate decarboxylase (2-keto acid decarboxylase) coding sequence from Lactococcus lactis (SEQ ID NO:180), and the alcohol dehydrogenase coding sequence from E. coli (YqhD) (SEQ ID NO:182).

Certain embodiments utilize polynucleotides that encode polypeptides associated with the biosynthesis of 4-hydroxybutyrate and/or 1,4-butanediol. For instance, certain embodiments employ one or more alpha-ketoglutarate decarboxylase encoding polynucleotides, including CCDC5180_{—}0513 (SEQ ID NO:199) from Mycobacterium bovis and SYNPCC7002_A2770 (SEQ ID NO:201) from Synechococcus sp PCC 7002. Some embodiments employ one or more 4-hydroxybutyrate dehydrogenase encoding polynucleotides, including PGN_{—}0724 (SEQ ID NO:203) from Porphyromonas gingivalis and CKR_{—}2662 (SEQ ID NO:205) from Clostridium kluyveri. Particular embodiments employ one or more succinyl-CoA synthetase encoding polynucleotides, such as the alpha subunit sucC (b0728) (SEQ ID NO:213) from E. coli and the beta subunit sucD (b0729) (SEQ ID NO:215) from E. coli. Some embodiments utilize one or more succinate-semialdehyde dehydrogenase encoding polynucleotides, such as PGTDC60_{—}1813 (SEQ ID NO:217) from Porphyromonas gingivalis. Examples of 4-hydroxybutyryl-CoA transferase encoding polynucleotides include cat2 (CKR_{—}2666) (SEQ ID NO:207) from Clostridium kluyveri, and homologs from Clostridium aminobutyricum and Porphyromonas gingivalis. Particular examples of aldehyde/alcohol dehydrogenase encoding polynucleotides include adhE2 (CEA_P0034) (SEQ ID NO:209) from Clostridium acetobutylicum and adhE (b1241) (SEQ ID NO:211) from E. coli.

Certain embodiments employ polynucleotides that encode polypeptides associated with the biosynthesis of polyamines or intermediates thereof. Examples include polynucleotides that encode an arginine decarboxylase to convert L-arginine into agmatine, an agmatine deiminase to convert agmatine into N-carbamoylputrescine, and an N-carbamoylputrescine amidase to convert N-carbamoylputrescine into putrescine. One example of an arginine decarboxylase encoding polynucleotide is Synpcc7942_{—}1037 (SEQ ID NO:219) from S. elongatus PCC7942. Specific examples of agmatine deiminase encoding polynucleotides include Synpcc7942_{—}2402 (SEQ ID NO:221) and Synpcc7942_{—}2461 from S. elongatus PCC7942. One exemplary N-carbamoylputrescine amidase encoding polynucleotide is Synpcc7942_{—}2145 (SEQ ID NO:223) from S. elongatus PCC7942.

Exemplary nucleotide sequences that encode the proteins and enzymes of the application encompass full-length reference polypeptides described herein (e.g., full-length acyl-ACP reductases, ACPs, glycyogen breakdown proteins, ACCases, DGATs, fatty acyl-CoA synthetases, aldehyde dehydrogenases, alcohol dehydrogenases), as well as portions of the full-length or substantially full-length nucleotide sequences of these genes or their transcripts or DNA copies of these transcripts. Portions of a nucleotide sequence may encode polypeptide portions or segments that retain the biological activity of the reference polypeptide. A portion of a nucleotide sequence that encodes a biologically active fragment of an enzyme provided herein may encode at least about 20, 21, 22, 23, 24, 25, 30, 40, 50, 60, 70, 80, 90, 100, 120, 150, 200, 300, 400, 500, 600, or more contiguous amino acid residues, almost up to the total number of amino acids present in a full-length enzyme. It will be readily understood that “intermediate lengths,” in this context and in all other contexts used herein, means any length between the quoted values, such as 101, 102, 103, etc.; 151, 152, 153, etc.; 201, 202, 203, etc.

The polynucleotides of the present invention, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a polynucleotide fragment of almost any length may be employed, with the total length preferably being limited by the ease of preparation and use in the intended recombinant DNA protocol.

The invention also contemplates variants of the nucleotide sequences of the polypeptides described herein (e.g., acyl-ACP reductases, ACPs, DGATs, glycogen breakdown proteins, fatty acyl-CoA synthetases, aldehyde dehydrogenases, alcohol dehydrogenases, ACCases). Nucleic acid variants can be naturally-occurring, such as allelic variants (same locus), homologs (different locus), and orthologs (different organism) or can be non naturally-occurring. Naturally occurring variants such as these can be identified and isolated using well-known molecular biology techniques including, for example, various polymerase chain reaction (PCR) and hybridization-based techniques as known in the art. Naturally occurring variants can be isolated from any organism that encodes one or more genes having an activity of interest, such as a glycogen synthesis/storage associated activity, glycogen breakdown activity, acyl-ACP reductase activity, ACP activity, DGAT, fatty acyl-CoA synthetase, aldehyde dehydrogenase, and/or an acetyl-CoA carboxylase activity. Embodiments of the present invention, therefore, encompass Cyanobacteria comprising such naturally occurring polynucleotide variants.

Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. In certain aspects, non-naturally occurring variants may have been optimized for use in Cyanobacteria, such as by engineering and screening the enzymes for increased activity, stability, or any other desirable feature. The variations can produce both conservative and non-conservative amino acid substitutions (as compared to the originally encoded product). For nucleotide sequences, conservative variants include those sequences that, because of the degeneracy of the genetic code, encode the amino acid sequence of a reference polypeptide. Variant nucleotide sequences also include synthetically derived nucleotide sequences, such as those generated, for example, by using site-directed mutagenesis but which still encode a biologically active polypeptide described herein, including but not limited to polypeptides having an acyl-ACP reductase activity, an ACP activity, glycogen breakdown activity, DGAT activity, fatty acyl-CoA synthetase activity, aldehyde dehydrogenase activity, alcohol dehydrogenase, and/or an acetyl-CoA carboxylase activity. Generally, variants of a particular reference nucleotide sequence will have at least about 30%, 40% 50%, 55%, 60%, 65%, 70%, generally at least about 75%, 80%, 85%, 90%, 95% or 98% or more sequence identity to that particular nucleotide sequence as determined by sequence alignment programs described elsewhere herein using default parameters.

Known reference nucleotide sequences can be used to isolate corresponding sequences and alleles from other organisms, particularly other microorganisms. Methods are readily available in the art for the hybridization of nucleic acid sequences. Coding sequences from other organisms may be isolated according to well known techniques based on their sequence identity with the coding sequences set forth herein. In these techniques all or part of the known coding sequence is used as a probe which selectively hybridizes to other reference coding sequences present in a population of cloned genomic DNA fragments or cDNA fragments (i.e., genomic or cDNA libraries) from a chosen organism.

Accordingly, the present invention also contemplates polynucleotides that hybridize to reference nucleotide sequences, or to their complements, under stringency conditions described below. As used herein, the term “hybridizes under low stringency, medium stringency, high stringency, or very high stringency conditions” describes conditions for hybridization and washing. Guidance for performing hybridization reactions can be found in Ausubel et al., (1998, supra), Sections 6.3.1-6.3.6. Aqueous and non-aqueous methods are described in that reference and either can be used.

Reference herein to “low stringency” conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6× sodium chloride/sodium citrate (SSC) at about 45° C., followed by two washes in 0.2×SSC, 0.1% SDS at least at 50° C. (the temperature of the washes can be increased to 55° C. for low stringency conditions).

“Medium stringency” conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C.

“High stringency” conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In certain embodiments, a acyl-ACP reductase, ACP, glycogen breakdown protein, aldehyde dehydrogenase, alcohol dehydrogenase, alcohol dehydrogenase, and/or acetyl-CoA carboxylase enzyme (or other enzyme described herein) is encoded by a polynucleotide that hybridizes to a disclosed nucleotide sequence under very high stringency conditions. One embodiment of very high stringency conditions includes hybridizing in 0.5 M sodium phosphate, 7% SDS at 65° C., followed by one or more washes in 0.2×SSC, 1% SDS at 65° C.

Other stringency conditions are well known in the art and the skilled artisan will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridization rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8).

In general, the Tₘ of a perfectly matched duplex of DNA may be predicted as an approximation by the formula: Tₘ=81.5+16.6 (log₁₀ M)+0.41 (% G+C)−0.63 (% formamide)−(600/length) wherein: M is the concentration of Na⁺, preferably in the range of 0.01 molar to 0.4 molar; % G+C is the sum of guano sine and cytosine bases as a percentage of the total number of bases, within the range between 30% and 75% G+C; % formamide is the percent formamide concentration by volume; length is the number of base pairs in the DNA duplex. The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In one example of a hybridization procedure, a membrane (e.g., a nitrocellulose membrane or a nylon membrane) containing immobilized DNA is hybridized overnight at 42° C. in a hybridization buffer (50% deionized formamide, 5×SSC, 5× Reinhardt's solution (0.1% fecal, 0.1% polyvinylpyrollidone and 0.1% bovine serum albumin), 0.1% SDS and 200 mg/mL denatured salmon sperm DNA) containing a labeled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.

Polynucleotides and fusions thereof may be prepared, manipulated and/or expressed using any of a variety of well established techniques known and available in the art. For example, polynucleotide sequences which encode polypeptides of the invention, or fusion proteins or functional equivalents thereof, may be used in recombinant DNA molecules to direct expression of a triglyceride or lipid biosynthesis enzyme in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence. Such nucleotides are typically referred to as “codon-optimized.”

Moreover, the polynucleotide sequences of the present invention can be engineered using methods generally known in the art in order to alter polypeptide encoding sequences for a variety of reasons, including but not limited to, alterations which modify the cloning, processing, expression and/or activity of the gene product.

In order to express a desired polypeptide, a nucleotide sequence encoding the polypeptide, or a functional equivalent, may be inserted into appropriate expression vector, i.e., a vector that contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. In certain embodiments, the polynucleotides of the present invention may be introduced and expressed in Cyanobacterial systems. As such, the present invention contemplates the use of vector and plasmid systems having regulatory sequences (e.g., promoters and enhancers) that are suitable for use in various Cyanobacteria (see, e.g., Koksharova et al. Applied Microbiol Biotechnol 58:123-37, 2002). For example, the promiscuous RSF1010 plasmid provides autonomous replication in several Cyanobacteria of the genera Synechocystis and Synechococcus (see, e.g., Mermet-Bouvier et al., Curr Microbiol 26:323-327, 1993). As another example, the pFC1 expression vector is based on the promiscuous plasmid RSF1010. pFC1 harbors the lambda cl857 repressor-encoding gene and pR promoter, followed by the lambda cro ribosome-binding site and ATG translation initiation codon (see, e.g., Mermet-Bouvier et al., Curr Microbiol 28:145-148, 1994). The latter is located within the unique Ndel restriction site (CATATG) of pFC1 and can be exposed after cleavage with this enzyme for in-frame fusion with the protein-coding sequence to be expressed.

The “control elements” or “regulatory sequences” present in an expression vector (or employed separately) are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. Generally, it is well-known that strong E. coli promoters work well in Cyanobacteria. Also, when cloning in Cyanobacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. Other vectors containing IPTG inducible promoters, such as pAM1579 and pAM2991trc, may be utilized according to the present invention.

Certain embodiments may employ a temperature inducible system or temperature inducible regulatory sequences (e.g., promoters, enhancers, repressors). As one example, an operon with the bacterial phage left-ward promoter (PO and a temperature sensitive repressor gene C1857 may be employed to produce a temperature inducible system for producing fatty acids and/or triglycerides in Cyanobacteria (see, e.g., U.S. Pat. No. 6,306,639, herein incorporated by reference). It is believed that at a non-permissible temperature (low temperature, 30 degrees Celsius), the repressor binds to the operator sequence, and thus prevents RNA polymerase from initiating transcription at the P_{L} promoter. Therefore, the expression of encoded gene or genes is repressed. When the cell culture is transferred to a permissible temperature (37-42 degrees Celsius), the repressor cannot bind to the operator. Under these conditions, RNA polymerase can initiate the transcription of the encoded gene or genes.

In Cyanobacterial systems, a number of expression vectors or regulatory sequences may be selected depending upon the use intended for the expressed polypeptide. When large quantities are needed, vectors or regulatory sequences which direct high level expression of encoded proteins may be used. For example, overexpression of ACCase enzymes may be utilized to increase fatty acid biosynthesis. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of (3-galactosidase so that a hybrid protein is produced; pIN vectors (Van Heeke & Schuster, J. Biol. Chem. 264:5503 5509 (1989)); and the like. pGEX Vectors (Promega, Madison, Wis.) may also be used to express foreign polypeptides as fusion proteins with glutathione S-transferase (GST).

Certain embodiments may employ Cyanobacterial promoters or regulatory operons. In certain embodiments, a promoter may comprise an rbcLS operon of Synechococcus, as described, for example, in Ronen-Tarazi et al. (Plant Physiology 18:1461-1469, 1995), or a cpc operon of Synechocystis sp. strain PCC 6714, as described, for example, in Imashimizu et al. (J Bacteriol. 185:6477-80, 2003). In certain embodiments, the tRNApro gene from Synechococcus may also be utilized as a promoter, as described in Chungjatupornchai et al. (Curr Microbiol. 38:210-216, 1999). Certain embodiments may employ the nirA promoter from Synechococcus sp. strain PCC7942, which is repressed by ammonium and induced by nitrite (see, e.g., Maeda et al., J. Bacteriol. 180:4080-4088, 1998; and Qi et al., Applied and Environmental Microbiology 71:5678-5684, 2005). The efficiency of expression may be enhanced by the inclusion of enhancers which are appropriate for the particular Cyanobacterial cell system which is used, such as those described in the literature.

In certain embodiments, expression vectors or introduced promoters utilized to overexpress an exogenous or endogenous acyl-ACP reductase, ACP, DGAT, fatty acyl-CoA synthetase, glycogen breakdown protein, aldehyde dehydrogenase, alcohol dehydrogenase, and/or acetyl-CoA carboxylase, or fragment or variant thereof, comprise a weak promoter under non-inducible conditions, e.g., to avoid toxic effects of long-term overexpression of any of these polypeptides. One example of such a vector for use in Cyanobacteria is the pBAD vector system. Expression levels from any given promoter may be determined, e.g., by performing quantitative polymerase chain reaction (qPCR) to determine the amount of transcript or mRNA produced by a promoter, e.g., before and after induction. In certain instances, a weak promoter is defined as a promoter that has a basal level of expression of a gene or transcript of interest, in the absence of inducer, that is ≦2.0% of the expression level produced by the promoter of the rnpB gene in S. elongatus PCC7942. In other embodiments, a weak promoter is defined as a promoter that has a basal level of expression of a gene or transcript of interest, in the absence of inducer, that is ≦5.0% of the expression level produced by the promoter of the rnpB gene is S. elongatus PCC7942.

It will be apparent that further to their use in vectors, any of the regulatory elements described herein (e.g., promoters, enhancers, repressors, ribosome binding sites, transcription termination sites) may be introduced directly into the genome of a photosynthetic microorganism (e.g., Cyanobacterium), typically in a region surrounding (e.g., upstream or downstream of) an endogenous or naturally-occurring gene such as an acyl-ACP reductase (e.g., orf1594 in Synechococcus elongatus), an ACP, or an ACCase, to regulate expression (e.g., facilitate overexpression) of that gene.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983). The presence of a desired polynucleotide, such as an acyl-ACP reductase, ACP, glycogen breakdown protein, and/or an acetyl-CoA carboxylase encoding polypeptide, may also be confirmed by PCR.

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Cyanobacterial host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct localization of the encoded polypeptide to a desired site within the cell. Other recombinant constructions may be used to join sequences encoding a polypeptide of interest to nucleotide sequence encoding a polypeptide domain which will direct secretion of the encoded protein.

In particular embodiments of the present invention, a modified photosynthetic microorganism of the present invention has reduced expression of one or more genes selected from glucose-1-phosphate adenyltransferase (glgC), phosphoglucomutase (pgm), and/or glycogen synthase (glgA). In particular embodiments, the modified photosynthetic microorganism comprises a mutation of one or more of these genes. Specific glgC, pgm, and glgA sequences may be mutated or modified, or targeted to reduce expression.

Examples of such glgC polynucleotide sequences are provided in SEQ ID NOs:28 (Synechocystis sp. PCC6803), 34 (Nostoc sp. PCC 7120), 33 (Anabaena variabilis), 32 (Trichodesmium erythraeum IMS 101), 27 (Synechococcus elongatus PCC7942), 30 (Synechococcus sp. WH8102), 31 (Synechococcus sp. RCC 307), and 29 (Synechococcus sp. PCC 7002), which respectively encode GlgC polypeptides having sequences set forth in SEQ ID NOs: 86, 87, 88, 89, 90, 91, 92, and 93.

Examples of such pgm polynucleotide sequences are provided in SEQ ID NOs: 25 (Synechocystis sp. PCC6803), 75 (Synechococcus elongatus PCC7942), 26 (Synechococcus sp. WH8102), 78 (Synechococcus RCC307), and 80 (Synechococcus 7002), which respectively encode Pgm polypeptides having sequences set forth in SEQ ID NOs:74, 76, 77, 79 and 81.

Examples of such glgA polynucleotide sequences are provided in SEQ ID NOs:36 (Synechocystis sp. PCC6803), 42 (Nostoc sp. PCC 7120), 41 (Anabaena variabilis), 40 (Trichodesmium erythraeum IMS 101), 35 (Synechococcus elongatus PCC7942), 38 (Synechococcus sp. WH8102), 39 (Synechococcus sp. RCC 307), and 37 (Synechococcus sp. PCC 7002), which respectively encode GlgA polypeptides having sequences set forth in SEQ ID NOs:94, 95, 96, 97, 98, 99, 100 and 101.

In particular embodiments of the present invention, a modified photosynthetic microorganism of the present invention has reduced expression of one or more endogenous aldehyde decarbonylases. One example of an aldehyde decarbonylase is encoded by orf1953 is S. elongatus PCC7942. Another example is an aldehyde decarbonylase encoded by orfsll0208 in Synechocystis sp. PCC6803. In particular embodiments, a modified photosynthetic microorganism of the present invention has reduced expression of one or more endogenous acyl-ACP synthetases (Aas). One example is encoded by Aas of S. elongatus PCC7942. In some embodiments, a modified photosynthetic microorganism has reduced expression of one or more endogenous aldehyde dehydrogenases. One example is encoded by orf0489 of Synechococcus elongatus PCC7942.

### EXAMPLES

### Example 1

### Generation of Glycogen Synthesis Mutants

Glycogen synthesis mutants (ΔglgC and ΔglgA) were prepared as described, for example, in U.S. Application No. 2010/0184169 and WO/2010/075440. Briefly, the glucose-1-phosphate adenylyltransferase gene (Synpcc7942_{—}0603, glgC) and the UDP-glucose-glycogen glucosyltransferase gene (Synpcc7942_{—}2518, glgA) in the S. elongatus PCC 7942 strain were individually inactivated by deletion to generate two different modified S. elongatus strains, ΔglgC and ΔglgA.

The ΔglgC and ΔglgA deletion strains were constructed as follows. Polymerase chain reaction was used to amplify genomic DNA regions flanking the glgC and glgA genes. Amplified upstream and downstream flanking regions were sequentially cloned upstream and downstream of the gentamicin resistance marker in plasmid pCRG. The pCRG plasmid is not capable of autonomous replication in Synechococcus.

The resulting plasmids were individually transformed into S. elongatus PCC 7942 using established methods. Following selection on gentamicin-containing medium, recombinant strains were propagated and their genomic DNA analyzed by PCR to verify deletion of the targeted gene in the respective ΔglgC and ΔglgA deletion strains. Strains, plasmids, primer sequences, vector construction, and primer sequences may be found in Tables C, D and E.

TABLE C

| Strain | Relevant phenotype or genotype |
|---|---|
| S. elongatus | |
| S. elongatus | Wild-type strain |
| PCC 7942 | |
| ΔglgC | Wild type derivative; deletion of 831 bp of glgC |
| | replaced by ~1 kb Gm^{r} cartridge |
| E. coli | |
| DH5α | supE44 ΔlacU169 (φ80 lacZΔM15) hsdR178 recA1 |
| | endA1 gyrA96 thi-1 relA1 |

TABLE D

| Plasmid | Construction |
|---|---|
| pAM2314 | Spec^{r}Strep^{r}, Ap^{r}; Neutral site 1 |
| | recombination vector |
| pAM2314FTlux | promoterless neutral site 1 recombination |
| | vector containing luxAB from V. harveyi |
| pAM2314FT_PnblA_luxAB | Neutral site 1 recombination vector |
| | containing luxAB under control of the |
| | nblA promoter |
| pAM2314FTtrc3_kgtP | Neutral site 1 recombination vector |
| | containing E. coli kgtP under control of |
| | the P_{trc} promoter |
| pAM1579 | Km^{r}, Ap^{r}; Neutral site 2 recombination |
| | vector |
| pAM1579Ftrc3_kgtP | Neutral site 2 recombination vector |
| | containing E. coli kgtP under control of |
| | the P_{trc} promoter |
| pAM1579F | Km^{r}, Ap^{r}; NdeI site of pAM1579 removed |
| | by fill-in and self-ligation |
| pAM1579F-glgC | Km^{r}, Ap^{r}; 1815 bp PCR product |
| | containing glgC and 522 bp upstream |
| | of glgC translational start cloned |
| | into EcoRV-XbaI sites of pAM1579F |
| pAM3558 | Gm^{r}; Neutral site 2 recombination vector |
| pCR2.1-TOPO | Km^{r}, Ap^{r}; TOPO cloning vector |
| pCRG | Km^{r}, Ap^{r}, Gm^{r}; 1012 bp fragment from |
| | pAM3558 cloned into TOPO site of |
| | pCR2.1-TOPO |
| pCRglgA | Km^{r}, Ap^{r}, Gm^{r}; 718 bp PCR fragment |
| | upstream glgC cloned into SpeI-HindIII |
| | sites of pCRG |
| pCRglgAB | Km^{r}, Ap^{r}, Gm^{r}; 720 bp PCR fragment |
| | downstream glgC cloned into NotI-XbaI |
| | sites of pCRglgA |

TABLE D

| Gene | Primer Paris | |
|---|---|---|
| glgA5 | TGAGCCAAGTTGCGGTGCAG | SEQ ID |
| | | NO: 225 |
| glgA3 | CGCGCACTAGTGGAGAGGTTGTAGGTC | SEQ ID |
| | TGAC | NO: 226 |
| glgB5 | GTAGCGCGGCCGCCCTCGGAGCTACGG | SEQ ID |
| | CACCAG | NO: 227 |
| glgB3 | CGCGGTCTAGATACCGGCATAGCGCAG | SEQ ID |
| | TAAG | NO: 228 |
| gent5b | CGATCTCCTGAAGCCAGGGC | SEQ ID |
| | | NO: 229 |
| gent3a | GGCGTTGTGACAATTTACCG | SEQ ID |
| | | NO: 230 |
| glgCUpEcoR | GTTGTTGATATCTGAGCCAAGTTGCGG | SEQ ID |
| Vnew | TGCAG | NO: 231 |
| glgCcompdo | GTTGTTTCTAGATTAGATCACCGTGTT | SEQ ID |
| wnXbaI | GTCGGGAATAACC | NO: 232 |
| glg1 | CGGCACCGAGACACCAATGC | SEQ ID |
| | | NO: 233 |
| glg2 | GCATTGCTTGAGAATGCAGC | SEQ ID |
| | | NO: 234 |
| gent5 | ACATAAGCCTGTTCGGTTCG | SEQ ID |
| | | NO: 235 |
| gent3 | TTAGGTGGCGGTACTTGGGTC | SEQ ID |
| | | NO: 236 |
| NS1inta | GTCGATATCTGGCACGGTGC | SEQ ID |
| | | NO: 237 |
| NS1intb | CATTTCCGATGAGGTCGGTTATC | SEQ ID |
| | | NO: 238 |
| NS2inta | GCGATCGCCGAAGACTGTGAC | SEQ ID |
| | | NO: 239 |
| NS2intb | CGTTGCCGTAGACCAGTTGCTC | SEQ ID |
| | | NO: 240 |
| luxA1 | ACACCTATTAGGTGCGACAG | SEQ ID |
| | | NO: 241 |
| luxA2 | CATGATCGACGGAGGTGATG | SEQ ID |
| | | NO: 242 |
| kgtP_seq_f2 | GAAGTATCTGGTAAATACTGCGGG | SEQ ID |
| | | NO: 243 |
| NS2_rev_a | ACCAATGCTGGGTAGTTCTC | SEQ ID |
| | | NO: 244 |
| nbIA | GCAATAATGCGGCCGCGGCGCTGCCTG | SEQ ID |
| promoter_ | GGAAAGTCAC | NO: 245 |
| fwd | | |
| nbIA | CATTGAACATATGAGCCTCCGGCACTG | SEQ ID |
| promoter_ | CAGATG | NO: 246 |
| rev | | |
| kgtP_F1_Nde | GCAATAATCATATGGCTGAAAGTACTG | SEQ ID |
| | TAAC | NO: 247 |
| kgtP_R1_BgI | CATTGAAAGATCTCTAAAGACGCATCC | SEQ ID |
| | CCTTC | NO: 248 |
| kgtP_int_F1 | CATTAGGCGTTGGTCTGTCGTATGCGG | SEQ ID |
| | TCGCTAATGCTATATTTG | NO: 249 |
| kgtP_int_R1 | CAAATATAGCATTAGCGACCGCATACG | SEQ ID |
| | ACAGACCAACGCCTAATG | NO: 250 |

### Example 2

### Glycogen Synthesis Mutants Maintain Photosynthetic Complexes During Nitrogen Starvation

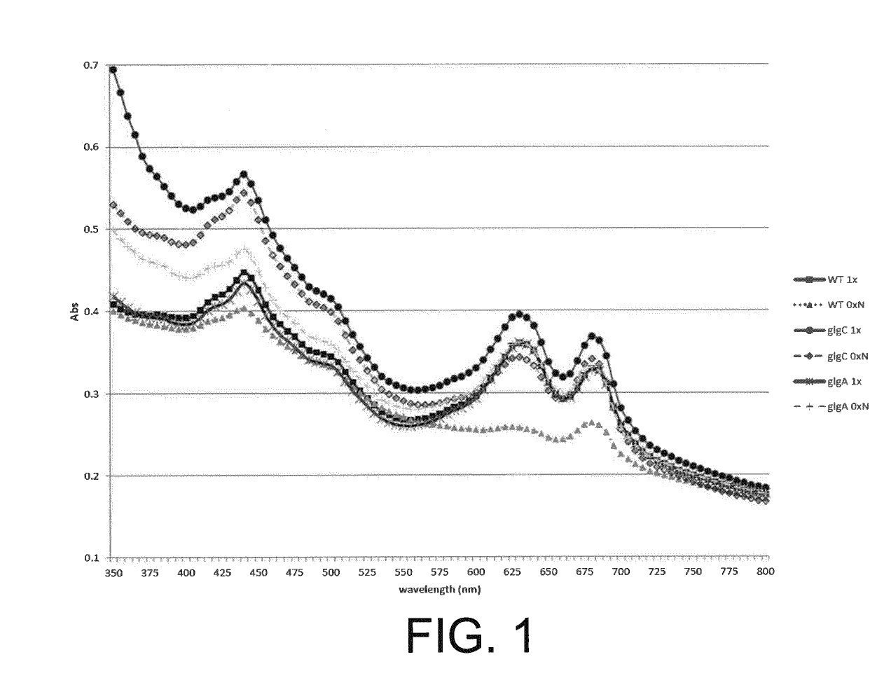

Glycogen synthesis mutants (ΔglgC and ΔglgA deletion strains) of S. elongatus PCC 7942 strain were tested for the presence of photosynthetic complexes during nitrogen starvation, relative to wild-type S. elongatus PCC 7942 strain. Wild-type S. elongatus, ΔglgC, and ΔglgA strains were cultured under conditions of nitrogen starvation, and measurements were take of photosynthetic complexes. The results are shown in FIG. 1, where photosynthetic complexes are lost during nitrogen starvation of wild-type S. elongatus, but are not degraded during starvation of glgC or glgA mutants. These data show that photosynthetic complexes are lost during nitrogen starvation of wild-type S. elongatus, but are not degraded during starvation of glgC or glgA mutants, indicating continued photosynthetic activity in glycogen synthesis mutants despite nutrient limitation. In one experiment, cultures of wild type (WT), ΔglgC and ΔglgA were resuspended in either nitrogen replete or 0×N media at a density of 0.25 OD750 and maintained in constant light (50 μE) at 300 C. After 24 hours in culture, spectral scans of each culture measuring absorbance from 350 to 800 nm were obtained to assess photosystem integrity. These data indicate that glycogen synthesis mutants maintain photosynthetic activity despite nutrient limitation.

NtcA-Transcriptional Activity.

One key aspect to the regulation of photosynthetic activity is the NtcA-mediated transcriptional response to nitrogen starvation. For instance, NtcA can bind to 2-oxoglutarate and regulate transcription of multiple genes, resulting in the degradation of photosynthetic complexes and the storage of carbon as glycogen. The possible suppression of NtcA-mediated transcriptional response was tested in the ΔglgC strain relative to wild-type.

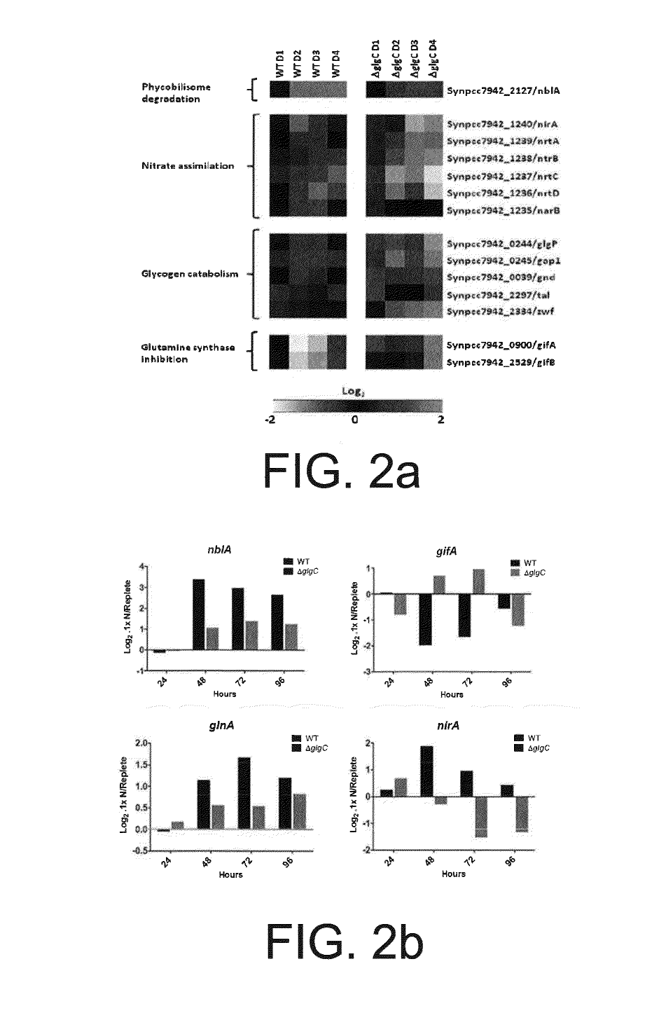

Wild-type S. elongatus and ΔglgC strains were cultured under conditions of nitrogen starvation, and transcriptome profiles were obtained for each cell culture. The results are shown in FIGS. 2a-2b, where transcriptome profiling shows suppression of the NtcA-mediated response in the glgC mutant, but not wild-type. With respect to FIG. 2a, wild type and ΔglgC were cultured in 1×N (Replete) or 0.1×N BG11. Total cellular RNA was isolated from each culture at 24 hour intervals for 4 days (D1-D4). Fluorescently labeled cDNA was hybridized to Nimblegen, PCC7942 v2 4x72K high-density gene expression arrays. The data is expressed as the log₂ ratio between the selected gene intensity from the 0.1×N BG11 sample and the gene intensity obtained from the time-matched, strain specific, nitrogen-replete control. The log₂ ratios (0.1×N/Replete) of selected NtcA-regulated, nitrogen-responsive transcripts are presented in this ordered gene cluster segmented by gene annotation and strain. WT=wild type, D1-D4 refers to days 1 to 4 in culture. With respect to FIG. 2b, wild type and ΔglgC were cultured in 1×N (Replete) or 0.1×N BG11. Total cellular RNA was isolated from each culture at 24 hour intervals for 4 days. Fluorescently labeled cDNA was hybridized to Nimblegen, PCC7942 v2 4x72K high-density gene expression arrays. The data is expressed as the log₂ ratio between the selected gene intensity from the 0.1×N BG11 sample and the gene intensity obtained from the time-matched, strain specific, nitrogen-replete control. The log₂ ratios (0.1×N/Replete) of NtcA-regulated, nitrogen-responsive transcripts nblA, gifA, glnA, and nirA are presented.

Suppression of the NtcA-mediated transcriptional response, which would otherwise degrade photosynthetic complexes, further indicates that glycogen synthesis mutants maintain photosynthetic activity despite nutrient limitation.

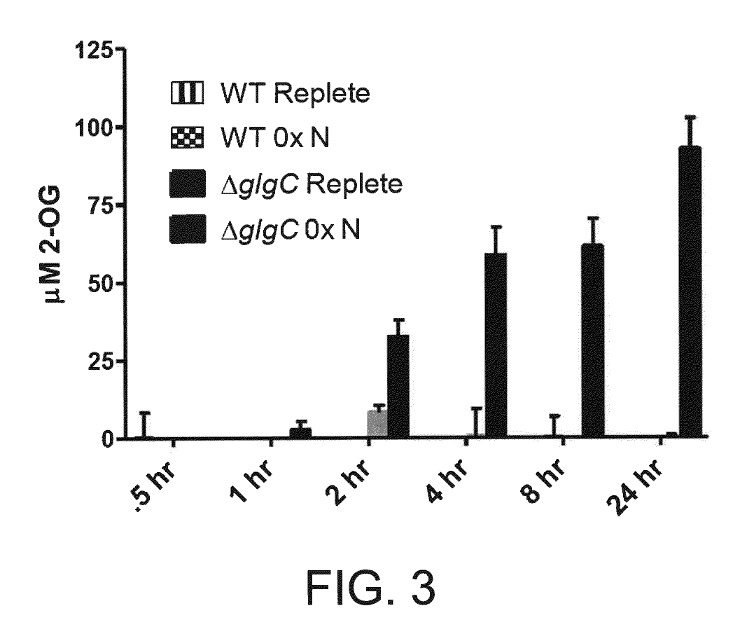

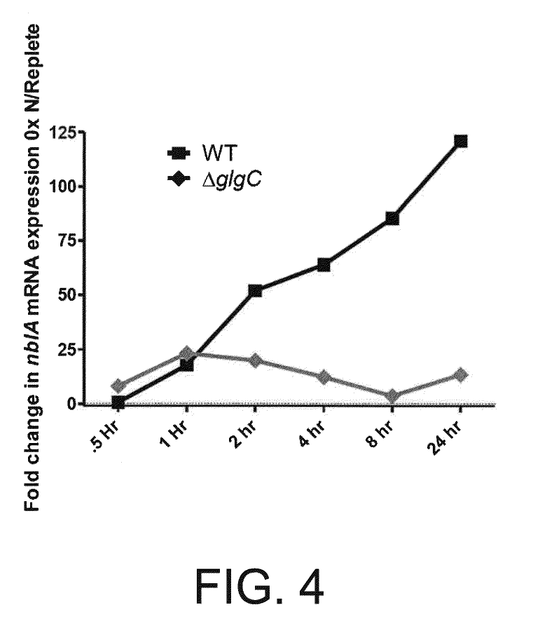

Quantitiative PCR was used to measure the induction and maintenance of nblA expression in response to nitrogen deprivation of both wild type and glgC null cells. While a significant increase in nblA transcript was detected in both wild type and glgC null cells one hour after nitrogen removal, the continued accumulation of nblA transcript detected in wild type by 24 hours did not occur in glgC null cells. Transcript levels for nblA in glgC null cells were actually less than one tenth that of wild type after 8 hours of nitrogen stress (FIG. 4). In one experiment, cultures were maintained in constant light at 30° C. and total cellular RNA was obtained from each culture at the indicated times after resuspension. RNA was converted to cDNA and relative nblA transcript levels were assessed by quantitative PCR. RNA input for each sample was normalized to rnpB, and nblA expression is presented as the fold change in expression compared to the time-matched, strain specific, nitrogen replete control. Moreover, 2-oxoglutarate accumulation and excretion from nitrogen starved ΔglgC coincided temporally with attenuation of nblA transcript levels in response to nitrogen deprivation (FIGS. 3 and 4). Furthermore, the initial induction of nblA transcript in nitrogen deprived glgC null cells was observed 30 minutes after nitrogen removal compared to 1 hour in wild type (FIG. 4). This faster induction of nblA indicates a significant difference in accumulation of 2-oxoglutarate in nitrogen starved glgC null compared to wild type cells.

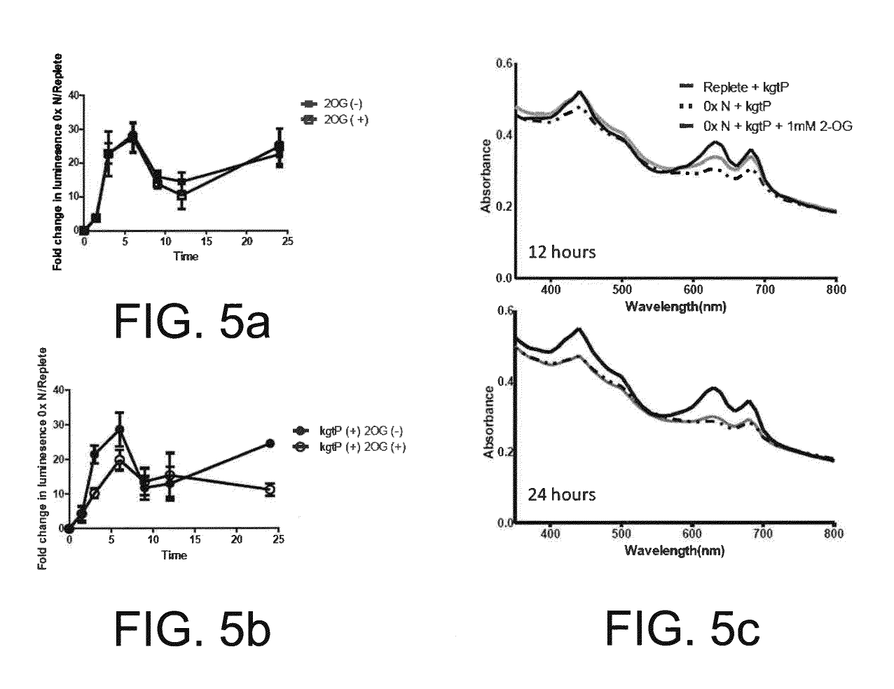

The strain WT^{nblA-lux×kgtP} was generated by transforming wild type S. elongatus with a translational fusion of the nblA promoter to a luxAB reporter system and the E. coli 2-oxoglutarate permease, kgtP placed under control of the IPTG inducible P_{trc} promoter. WT^{nblA-lux×kgtP} served as a tool in which the activity of the nblA promoter in response to nitrogen deprivation could be monitored in the presence and absence of 2-oxoglutarate internalization. While the expression of KgtP did not alter the activity of nblA-luxAB in response to nitrogen deprivation (FIGS. 5a and 5b) incubation of IPTG-induced WT^{nblA-lux×kgtP} in 0×N media containing 1 mM 2-oxoglutarate delayed the induction of nblA-luxAB, reduced the magnitude of nblA-luxAB luminescence (FIG. 5b), and delayed the onset of phycobilisome degradation (FIG. 5c). In one experiment, cultures in triplicate of (FIG. 5a) wild type WT^{nblA-lux} and (FIG. 5b) WT^{nblA-lux×kgtP} were seeded at an OD_{750/ml} of 0.4 and were maintained in constant light (50 μE) at 30° C. for 24 hours in replete media buffered at pH 7.5 with 20 mM HEPES containing 1 mM IPTG. At the end of the 24 hour induction period cultures were pelleted and resuspended to an OD_{750/ml} of 0.3 in HEPES buffered media into the following three conditions: 1) Replete media+1 mM IPTG, 2) 0×N media+1 mM IPTG, 3) 0×N media+1 mM IPTG and 1 mM α-ketoglutaric acid potassium salt (20G). Luminescence was measured at the indicated times using culture samples normalized to 0.2 OD_{750/ml}. Data is presented as the average fold change in luminescence (0×N/replete). Errors bars represent standard deviation of the mean fold change in Luminescence. FIG. 5c shows spectral scans of kgtP expressing cultures measuring absorbance from 350 to 800 nm were obtained to assess photosystem integrity at 12 and 24 hours after nitrate removal.

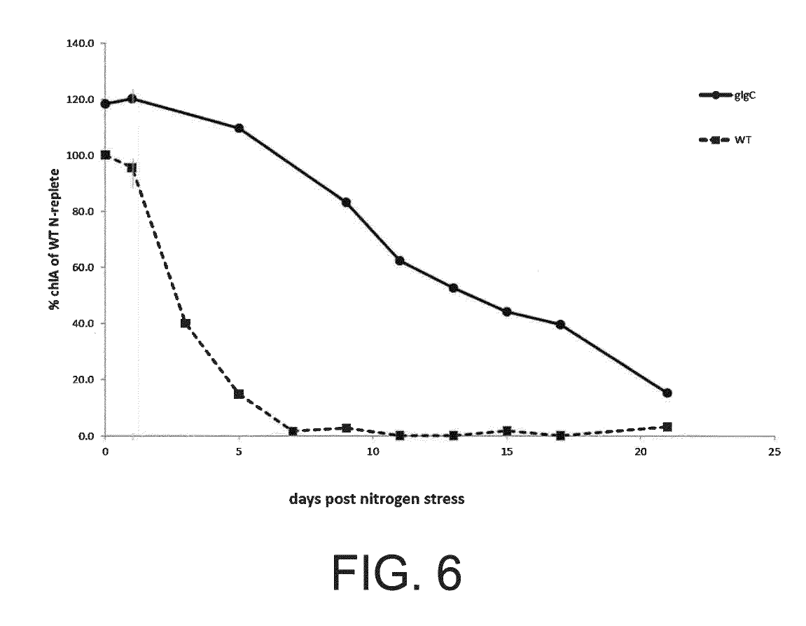

Chlorophyll A (chlA) levels were determined by the absorbance of cultures at 680 nm wavelength normalized to the optical density of the culture read at 750 nm (680 nm/750 nm). Relative chlA levels were then determined by setting WT under nitrogen replete growth conditions at day 0 as 100% chlA. The results are shown in FIG. 6, where wild-type S. elongatus showed near total loss of chlA by about 1 week of culture under nitrogen starvation, and ΔglgC strain maintained at least 20% chlA levels (relative to WT under non-stress conditions) even after three weeks. In one experiment, cultures of wild type (WT) and ΔglgC were resuspended in either nitrogen replete or 0×N media at a density of 0.25 OD₇₅₀ and maintained in constant light (50 μE) at 30° C. Spectral scans of each culture measuring absorbance from 350 to 800 nm were obtained to assess chlorophyll a levels at the indicated times. Data is expressed as the % of wild type chlorophyll a levels under non-stress (nitrogen replete) conditions.

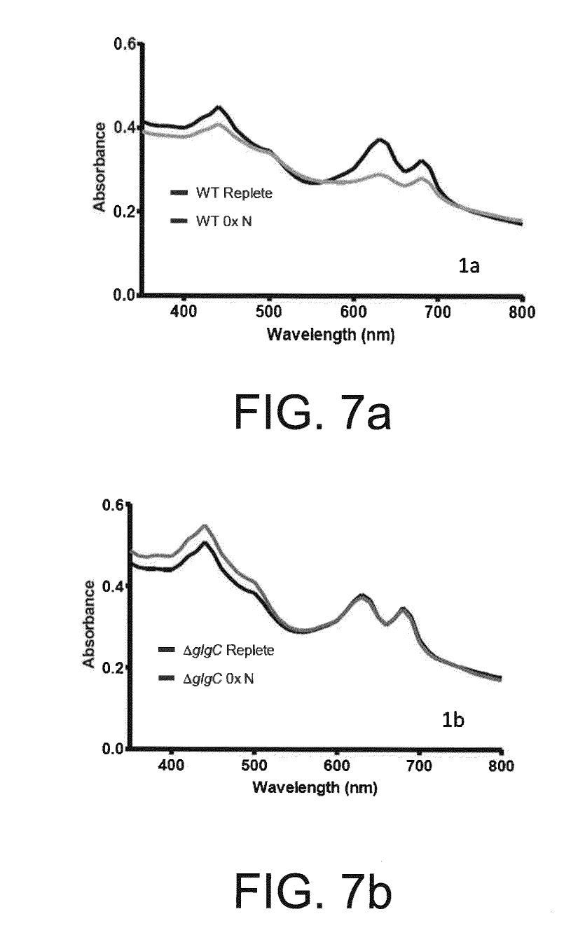

An S. elongatus mutant lacking the glgC gene (Synpcc7942_{—}0603) was generated to examine the significance of glycogen synthesis as a component of the global nitrogen starvation response. Growth of the wild type in nitrogen-deficient media triggered chlorosis as evidenced by a significant reduction in chlorophyll a and phycobiliprotein content by 24 hours (FIGS. 7a-7b). Chlorosis in the glgC mutant was severely impaired after 24 hours of nitrogen starvation (FIGS. 7a-7b). Furthermore, while 4 days of nitrogen stress induced the complete loss of both chlorophyll a and phycobiliprotein in wild type, it took twice as long for nitrogen starvation to render the glgC mutant completely chlorotic. In one experiment, cultures of wild type (WT) (FIG. 7a) and ΔglgC (FIG. 7b) were resuspended in either nitrogen replete or 0×N media at a density of 0.25 OD₇₅₀ and maintained in constant light (50 μE) at 30° C. After 24 hours in culture, spectral scans of each culture measuring absorbance from 350 to 800 nm were obtained to assess photosystem integrity.

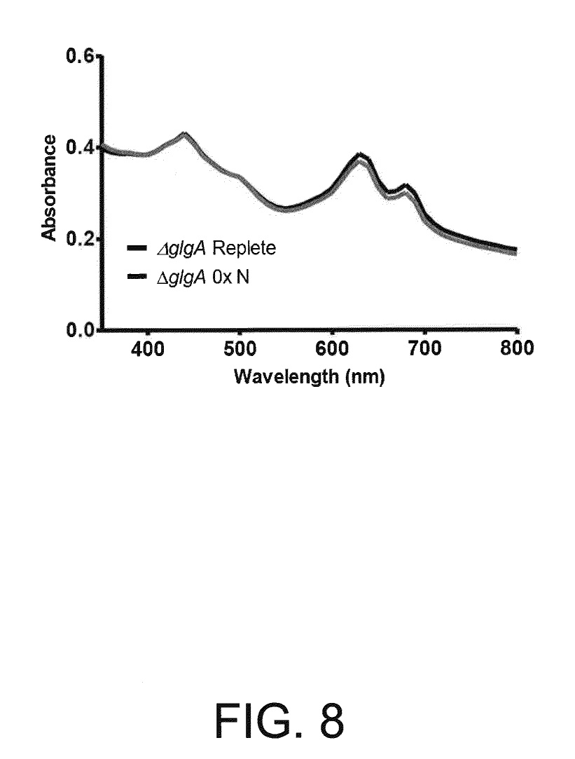

Attenuation of chlorosis was not specific to the glgC mutant as a glgA mutant lacking glycogen synthase activity exhibited a similar non-bleaching phenotype in response to nitrogen starvation (FIG. 8). In one experiment, An actively growing culture of glgA null cells (ΔglgA) was resuspended in either replete or 0×N media at a density of 0.25 OD750 and maintained in constant light (50 μE) at 300 C. After 24 hours in culture, spectral scans of each culture measuring absorbance from 350 to 800 nm were obtained to assess photosystem integrity.

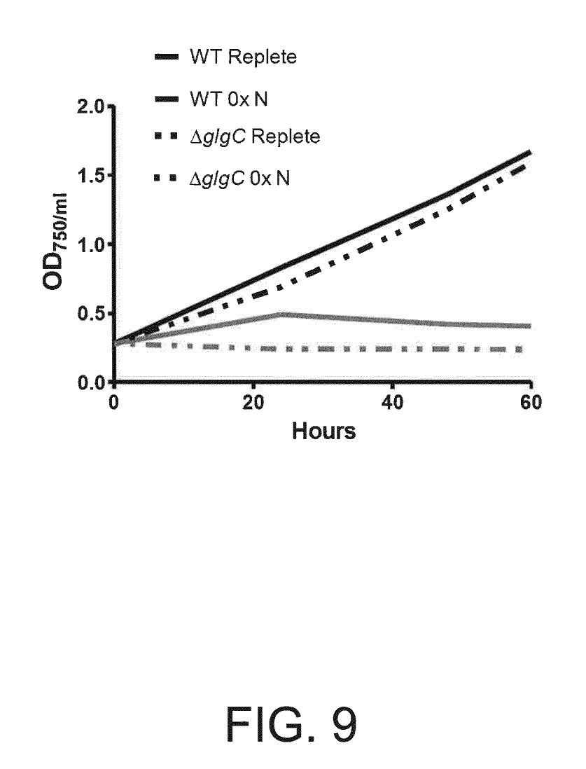

Growth of the wild type continued for approximately one doubling in nitrogen-deficient media after which growth stopped (FIG. 9). However, growth arrest of glgC null cells occurred immediately upon transfer into nitrogen-deficient media (FIG. 9) as did the growth of glgA null cells starved for nitrogen. In one experiment, cultures of wild type (WT) and ΔglgC were resuspended in either nitrogen replete or 0×N media at a density of 0.25 OD₇₅₀ and maintained in constant light (50 μE) at 30° C. The growth of each culture was assessed at the times indicated by measuring culture absorbance at 750 nm.

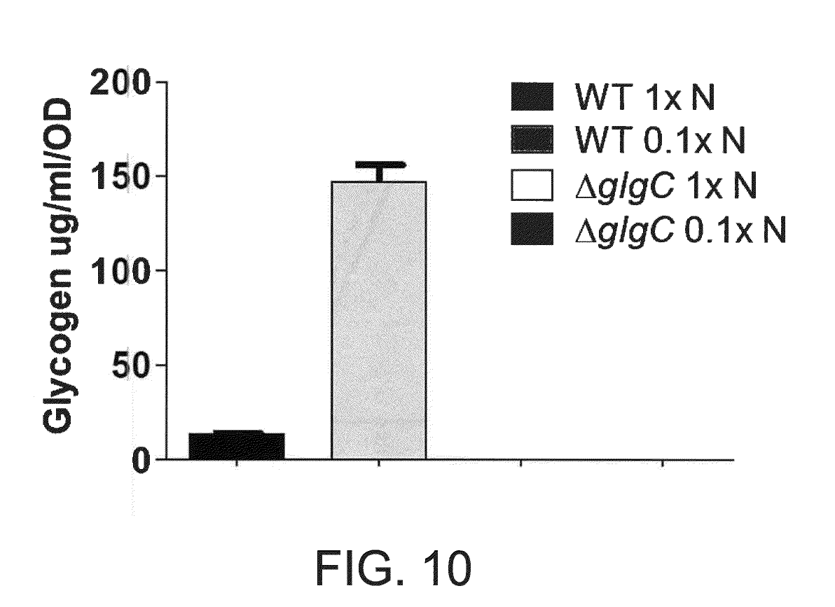

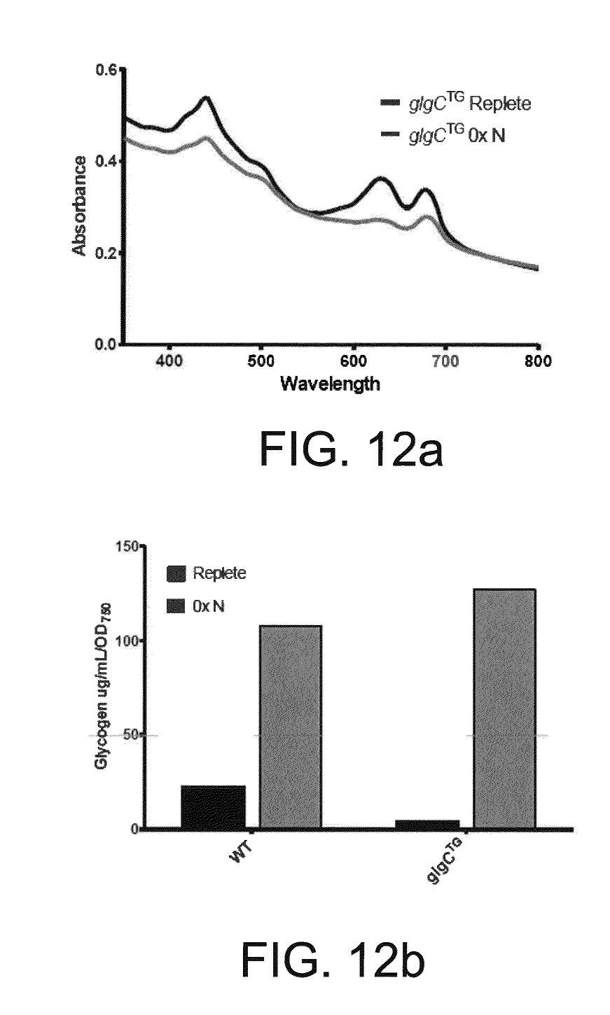

In addition, wild type accumulated approximately 50% of its dry weight as glycogen when cultured in nitrogen limited media, while glycogen was not detected in the glgC mutant (FIG. 10). An actively growing culture of glgC null cells (ΔglgC) was resuspended in either nitrogen replete (1×N) or nitrogen limited (0.1×N) media at a density of 0.3 OD750 and maintained in constant light (50 μE) at 300 C. Glycogen levels were assayed after 6 days in culture. The amount of glycogen (4) normalized to cell OD750 is shown. Moreover, the 50% decrease in cellular protein levels observed in nitrogen starved wild type, did not occur in the glgC mutant cells confirming the maintenance of phycobilisomes in the mutant which can constitute up to half of all cellular protein. Notably, phycobilisome maintenance did not have a positive impact on PSII activity as nitrogen stress triggered a similar drop in O₂ evolution rates in both WT and glgC null cells (FIG. 11). In one experiment, cells were grown and washed as described but were resuspended in either BG11 or 0×N BG11 to OD₇₅₀ of 0.35. For oxygen measurements, samples were diluted to OD₇₅₀ of 0.35 in 2 ml in BG11 or 0×N BG11 and supplemented with 10 mM sodium bicarbonate. The sample was sealed in a 1 cm cuvette and illuminated at room temperature with a LED white light source at a PPFD of 50 μmol/m²/s. Oxygen evolution was detected using a NeoFox fluorescent oxygen measuring system (Ocean Optics). A linear increase in oxygen concentration was observed, and the slope of this line was divided by 0.35 to determine oxygen evolution per OD₇₅₀. These phenotypes were complemented by expression of glgC recombined into neutral site 2 (FIGS. 12a-12b). In one experiment, actively growing cultures of wild type (WT) and ΔglgC carrying a glgC transgene (glgC^{TG}) were resuspended in either nitrogen replete or 0×N media at a density of 0.25 OD₇₅₀ and maintained in constant light (50 μE) at 30° C. After 24 hours, spectral scans were obtained (FIG. 12a) and glycogen levels (FIG. 12b) measured for each culture.

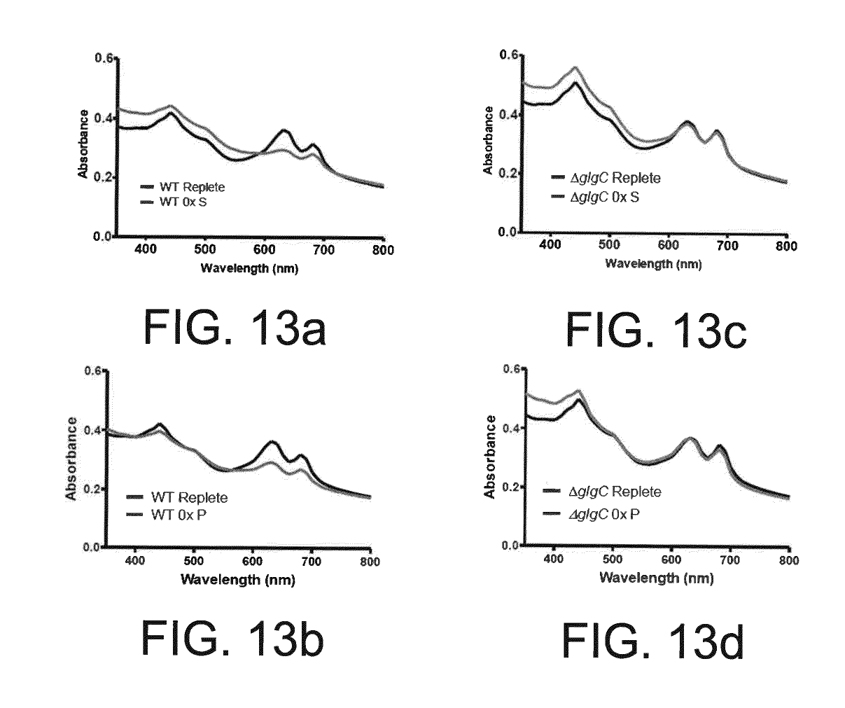

The chlorotic response of the glgC mutant to both sulfur and phosphate stress was evaluated while NtcA is not required for phycobilisome degradation under these stress conditions. Notably, glgC null cells exhibited a non-bleaching phenotype in response to both sulfur and phosphate stress (FIGS. 13a-13d). In one experiment, actively growing cultures of wild type (WT) and glgC null cells (ΔglgC) were resuspended in replete media or media without either sulfur (0×S) or phosphate (0×P) at a density of 0.25 OD₇₅₀ and maintained in constant light (50 μE) at 30° C. Spectral scans were obtained 24 hours after initiation of sulfur starvation and 120 hours after initiation of phosphate starvation.

### Example 3

### Glycogen Synthesis Mutants Secrete Carbon Skeletons and Produce Metabolites that are Convertible into Biofuel End-Products During Nitrogen Starvation

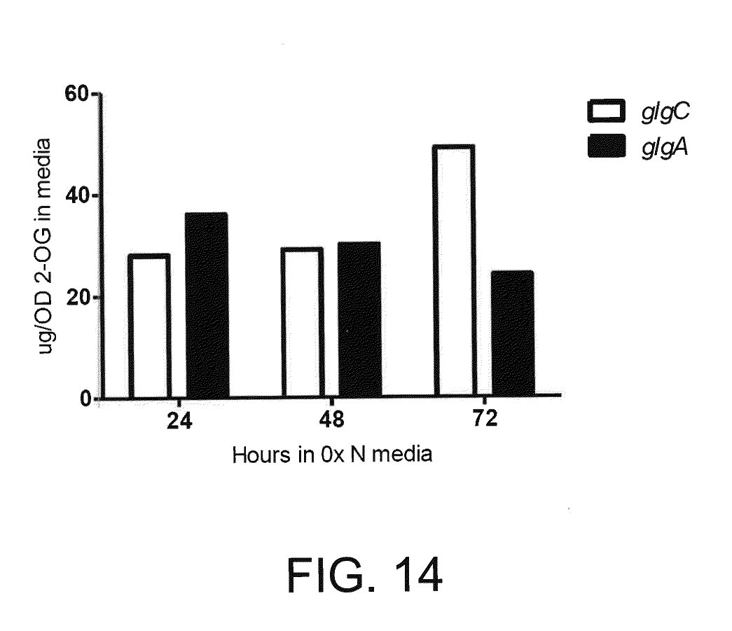

Glycogen synthesis mutants were tested for their ability to secrete carbon skeletons and produce metabolites that are convertible into biofuel end-products during nitrogen starvation. In one experiment, ΔglgC and ΔglgA deletion strains were cultured under conditions of nitrogen starvation and tested for the secretion of 2-oxoglutarate. The results are shown in FIG. 14, which shows that significant levels of 2-oxoglutarate are secreted each day by the glgC and glgA mutants post nitrogen starvation. In the experiment, cultures of wild type (WT), ΔglgC and ΔglgA were resuspended in either nitrogen replete (1×N) or media depleted entirely of nitrate (0×N) and maintained in constant light at 30° C. for 24, 48 and 72 hours. 2-oxoglutarate level (μg/OD) in the culture media was monitored for each sample by enzymatic assay at the specified times.

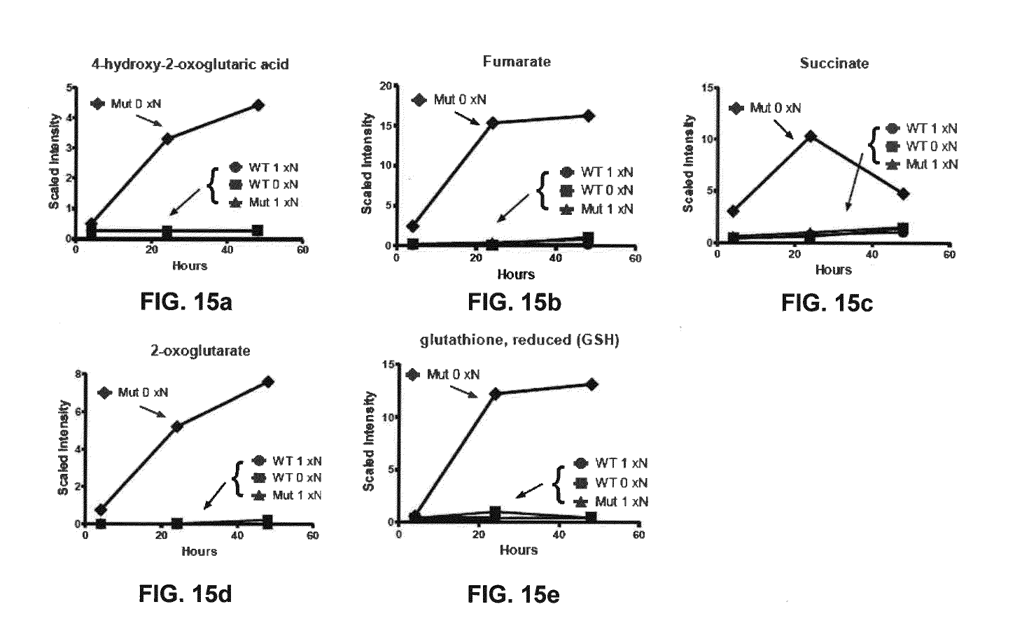

In another experiment, the wild-type S. elongatus and ΔglgC deletion strain were cultured under conditions of nitrogen starvation and tested for secretion of carbon skeletons fumarate, α-ketoglutarate, succinate, reduced glutathione (GSH), and 4-hydroxy-2-oxoglutaric acid. The results are shown in FIGS. 15a-15e, where the ΔglgC strain under nitrogen starvation (Mut 0×) showed significantly increased secretion of each carbon skeleton, relative to wild-type (Wt 1×) and the ΔglgC strain (Mut 1×) under normal conditions, and the wild-type under nitrogen starvation (Wt 0×). In this experiment, cultures of wild type (WT) and ΔglgC (Mut) were resuspended in either nitrogen replete (1×N) or media depleted entirely of nitrate (0×N) and maintained in constant light at 30° C. for 48 hours. Culture supernatant samples were taken 4, 24, and 48 hours after media transfer and prepared by centrifugation and aspiration of the supernatant. Mass spectrometry was used to determine relative levels of metabolites in culture supernatants. Shown are the relative levels of 2-oxoglutarate, fumarate and succinate in culture supernatants over time.

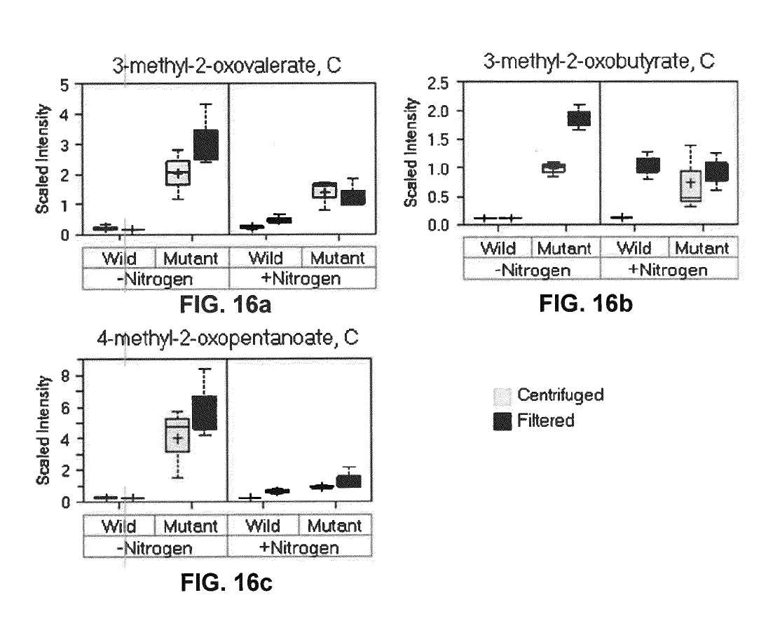

In another experiment, the wild-type S. elongatus and ΔglgC deletion strain were cultured under conditions of nitrogen starvation and tested for the production of metabolites such as 3-methyl-2-oxovalerate, 3-methyl-2-oxobutyrate, and 4-methyl-2-oxopentanoate, which are convertible into volatile 4- and 5-carbon alcohols that can serve as biofuel end-products. Samples were either centrifuged or filtered prior to testing. The results are shown in FIGS. 16a-16c, where ΔglgC deletion strain (mutant) under conditions of nitrogen starvation produced increased levels of each metabolite tested, relative to wild-type under any conditions (+Nitrogen, −Nitrogen), and relative to the ΔglgC mutant under normal conditions (+Nitrogen). In the experiment, cultures of wild type and ΔglgC were resuspended in either nitrogen replete (1×N) or media depleted entirely of nitrate (0×N). The cultures were maintained in constant light at 30° C. for 4 hrs at which time cells were snap frozen after being harvested either by harvested or after fast filtration in the light. Frozen samples were extracted and subjected to metabolite analysis. Data is normalized to protein in each sample and scaled relative to the median of the specified metabolite in all samples. Box plot details: mean value (+), median value (−), error bars indicate maximum and minimum of data distribution, box plot top and bottom denotes upper and lower quartile of the data.

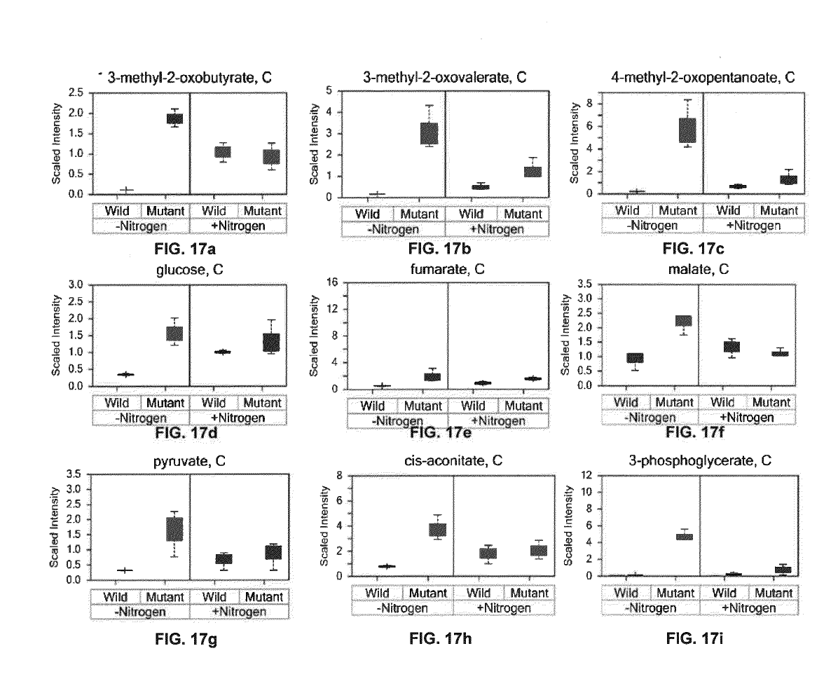

In another experiment, the wild-type S. elongatus and ΔglgC deletion strain were cultured under conditions of nitrogen starvation and tested for the production of metabolites such as 3-methyl-2-oxobutyrate, 3-methyl-2-oxovalerate, 4-methyl-2-oxopentanoate, glucose, fumarate, malate, pyruvate, cis-aconitatate and 3-phosphoglycerate. The results are shown in FIGS. 17a-17i, where ΔglgC deletion strain (mutant) under conditions of nitrogen starvation produced increased levels of each metabolite tested, relative to wild-type under any conditions (+Nitrogen, −Nitrogen), and relative to the ΔglgC mutant under normal conditions (+Nitrogen). In the experiment, cultures of wild type and ΔglgC were resuspended in either nitrogen replete (1×N) or media depleted entirely of nitrate (0×N). The cultures were maintained in constant light at 30° C. for 4 hrs at which time cells were snap frozen after being harvested by fast filtration in the light. Frozen samples were extracted and subjected to metabolite analysis. Data is normalized to protein in each sample and scaled as box plots relative to the median of the specified metabolite in all samples. Box plot details: mean value (+), median value (−), error bars indicate maximum and minimum of data distribution, box plot top and bottom denotes upper and lower quartile of the data.

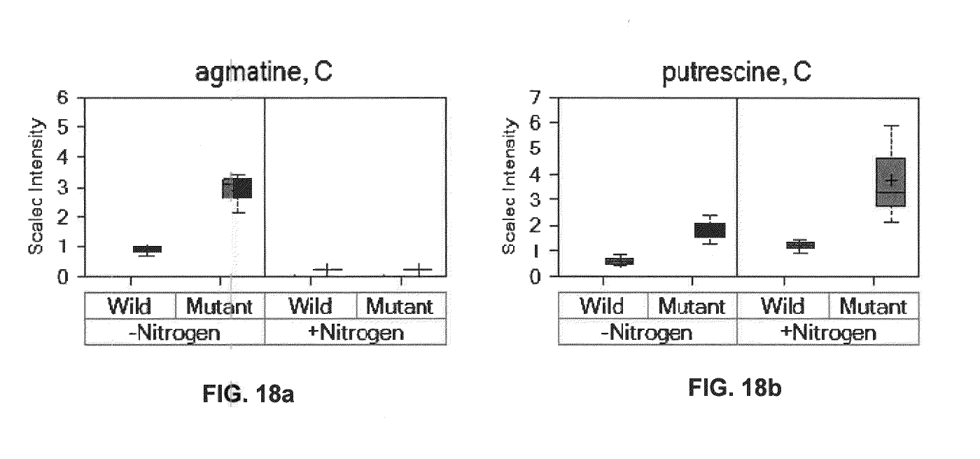

In another experiment, the wild-type S. elongatus and ΔglgC deletion strain were cultured under conditions of nitrogen starvation and tested for the production of polyamine intermediates such as agmatine and putrescine. The results are shown in FIGS. 18a-18b, where ΔglgC deletion strain (mutant) under conditions of nitrogen starvation produced increased levels of agmatine and putrescine relative to wild-type. In the experiment, cultures were maintained in constant light at 30° C. for 4 hrs at which time cells were snap frozen after being harvested by fast filtration in the light. Frozen samples were extracted and subjected to metabolite analysis. Data is normalized to protein in each sample and scaled as box plots relative to the median of the specified metabolite in all samples. Box plot details: mean value (+), median value (−), error bars indicate maximum and minimum of data distribution, box plot top and bottom denotes upper and lower quartile of the data.

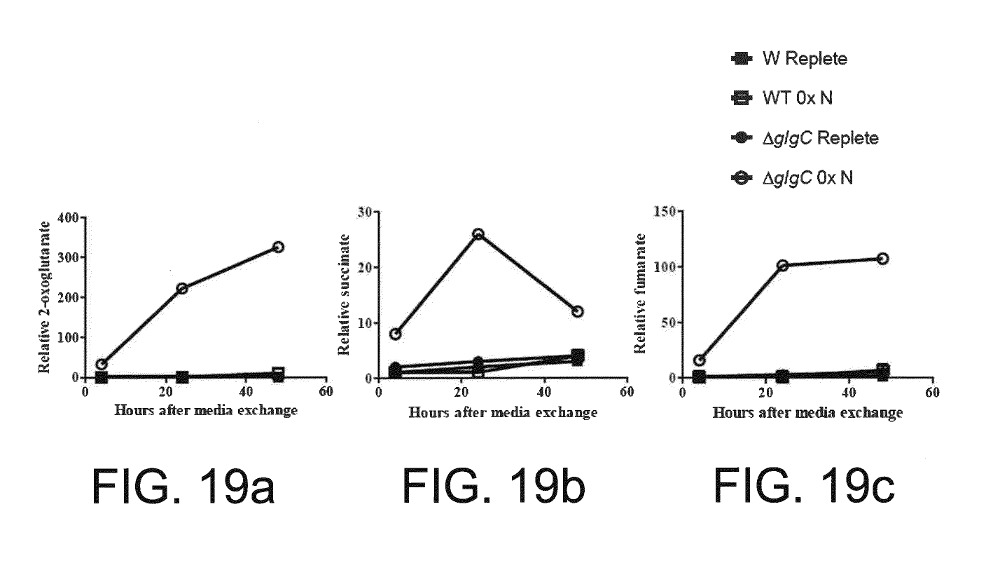

In another experiment, the metabolite profiles of media obtained from both wild type and ΔglgC cultures grown in the presence or absence of nitrogen were analyzed using mass spectrometry. TCA cycle intermediates succinate, fumarate, and 2-oxoglutarate were observed to accumulate in ΔglgC culture supernatants after transfer to nitrogen free media, with 2-oxoglutarate exhibiting the largest relative change in abundance (FIGS. 19a-19c). In the experiment, cultures of wild type (WT) and ΔglgC were resuspended in either nitrogen replete (1×N) or media depleted entirely of nitrate (0×N) and maintained in constant light at 30° C. for 48 hours. The culture supernatant samples were taken 4, 24, and 48 hours after media transfer and prepared by centrifugation and aspiration of the supernatant. Mass spectrometry was used to determine relative levels of metabolites in culture supernatants. Shown are the relative levels of 2-oxoglutarate, fumarate and succinate in culture supernatants over time. In addition, extracellular 2-oxoglutarate was quantitated at discrete time points following nitrogen starvation. Extracellular accumulation of 2-oxoglutarate from nitrogen starved glgC null cells was first detected 2 hours after nitrogen removal and increased from 30 μM at 2 hours to 90 μM by 24 hours of nitrogen starvation (FIG. 3). In one experiment, cultures of wild type (WT) and ΔglgC were resuspended in either nitrogen replete (1×N) or media depleted entirely of nitrate (0×N) and maintained in constant light at 30° C. for 24 hours. 2-oxoglutarate level (μM) in the culture media was monitored for each sample by enzymatic assay at the specified times. In FIG. 3, data was generated in triplicate and error is expressed as standard deviation of the mean. Samples whose mean fell below the below the 1 μM detection limit of the assay are not graphed.

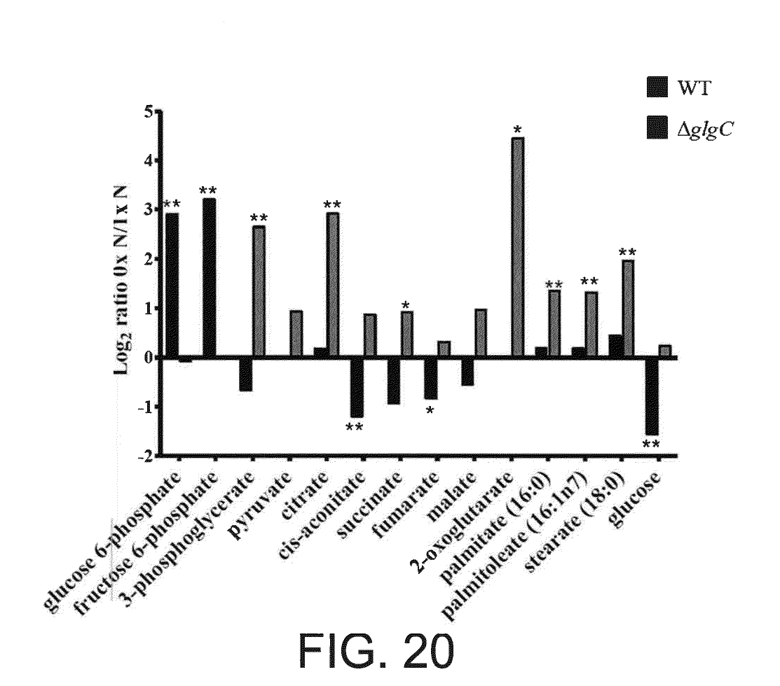

In another experiment, mass spectrometry was used to generate metabolic profiles of actively growing wild type and ΔglgC cultures transferred into nitrogen replete or nitrogen deprived media. Relative amounts of intracellular metabolites were determined for wild type and ΔglgC samples 4 hours after media transfer to monitor metabolites immediately after initiation of 2-oxoglutarate excretion. In wild type cells, glucose-6-phosphate and fructose-6-phosphate exhibited a significant increase while glucose levels decreased 4 hours after nitrogen removal compared to nitrogen replete samples (FIG. 20). In the experiment, cultures of wild type and ΔglgC were resuspended in either nitrogen replete (1×N) or media depleted entirely of nitrate (0×N). The cultures were maintained in constant light at 30° C. for 4 hrs at which time cells were harvested, snap frozen and subjected to metabolite analysis. Shown are the Logₑ ratios of indicated metabolites from nitrogen starved cells versus nitrogen replete cells. Metabolites without a reported ratio for the indicated strain were below the detection limit in at least two out of three biological replicates for at least one growth condition. [*]=ratio with a p-value 0.05, [**]=ratio with a p-value 0.01.

These results show that glycogen synthesis mutant strain(s) maintain photosynthetic capacity even under conditions of nutrient limitation, and can be used, for example, in continuous production systems under such conditions to generate internally accumulating and/or secreted metabolites suitable for conversion to, for example, volatile biofuel products.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not necessarily limited by the disclosure.

### EXEMPLARY EMBODIMENTS

The following are exemplary embodiments of the invention.

1. A system for producing a carbon-containing compound, comprising:

(a) a modified photosynthetic microorganism that accumulates a reduced amount of glycogen as compared to the wild-type photosynthetic microorganism; and

(b) a culture system for culturing said modified photosynthetic microorganism under a stress condition,

wherein said modified photosynthetic microorganism maintains photosynthetic activity and accumulates reduced biomass when grown under said stress condition as compared to when grown under non-stress conditions.

2. The system of embodiment 1, wherein said modified photosynthetic microorganism secretes and/or intracellularly accumulates an increased amount of a carbon-containing compound when grown under said stress condition as compared to when grown under non-stress conditions, or as compared to a corresponding wild-type microorganism grown under said stress condition.

3. The system of embodiment 1 or 2, wherein said modified photosynthetic microorganism:

(a) has reduced expression of one or more genes of a glycogen biosynthesis or storage pathway as compared to the wild-type photosynthetic microorganism;

(b) comprises one or more introduced polynucleotides encoding a protein that increases glycogen breakdown; and/or

(c) comprises one or more introduced polynucleotides encoding a protein that increases secretion of a glycogen precursor.

4. The system of any one of embodiments 1-3, wherein said stress condition is a reduced level of an essential nutrient.

5. The system of embodiment 4, wherein said essential nutrient is selected from at least one of nitrogen, sulfur, and phosphorous.

6. A method for producing a carbon-containing compound other than glycogen, comprising culturing in a culture media under a stress condition a modified photosynthetic microorganism that accumulates a reduced amount of glycogen as compared to the wild-type photosynthetic microorganism, wherein said modified photosynthetic microorganism maintains photosynthetic activity and accumulates reduced biomass when grown under said stress condition as compared to when grown under non-stress conditions.

7. The method of embodiment 6, wherein said modified photosynthetic microorganism secretes and/or intracellularly accumulates an increased amount of one or more carbon-containing compounds when grown under said stress condition as compared to when grown under non-stress conditions, or as compared to a corresponding wild-type microorganism grown under said stress condition.

8. The method of embodiment 6 or 7, wherein said modified photosynthetic microorganism:

(a) has reduced expression of one or more genes of a glycogen biosynthesis or storage pathway as compared to the wild-type photosynthetic microorganism;

(b) comprises one or more introduced polynucleotides encoding a protein that increases glycogen breakdown or secretion, and/or

(c) comprises one or more introduced polynucleotides encoding a protein that increases secretion of a glycogen precursor.

9. The method of any one of embodiments 6-8, further comprising harvesting said culture media after said modified photosynthetic organism has been cultured under said stress condition.

10. The method of any one of embodiments 6-9, further comprising obtaining said carbon-containing compound from said harvested culture media.

11. The method of any one of embodiments 6-10, further comprising harvesting said modified photosynthetic microorganism after it has been cultured under said stress condition.

12. The method of embodiment 11, further comprising obtaining said carbon-containing compound from said harvested modified photosynthetic microorganism.

13. The method of any one of embodiments 6-12, wherein said stress condition is a reduced level of an essential nutrient.

14. The method of embodiment 13, wherein said essential nutrient is selected from at least one of nitrogen, sulfur, and phosphorous.

15. The system of embodiment 3 or the method of claim 8, wherein said modified photosynthetic microorganism has reduced expression of one or more genes of a glycogen biosynthesis or storage pathway as compared to the wild-type photosynthetic microorganism, and wherein said one or more genes are selected from the group consisting of: a glucose-1-phosphate adenyltransferase (glgC) gene, a phosphoglucomutase (pgm) gene, and a glycogen synthase (glgA) gene.

16. The system or method of embodiment 15, wherein said one or more genes comprise a complete or partial gene deletion.

17. The system or method of any one of embodiments 1-16, wherein said photosynthetic microorganism is a Cyanobacterium.

18. The system or method of embodiment 17, wherein said Cyanobacterium is a Synechococcus elongatus.

19. The system or method of embodiment 18, wherein the Synechococcus elongatus is strain PCC 7942.

20. The system or method of embodiment 19, wherein the Cyanobacterium is a salt tolerant variant of Synechococcus elongatus strain PCC 7942.

21. The system or method of embodiment 17, wherein said Cyanobacterium is Synechococcus sp. PCC 7002.

22. The system or method of embodiment 17, wherein said Cyanobacterium is Synechocystis sp. PCC 6803.

23. The system or method of any one of embodiments 1-22, wherein said carbon-containing compound is a lipid.

24. The system or method of embodiment 23, wherein said lipid is a fatty acid, optionally a free fatty acid, a triglyceride, a wax ester, a fatty alcohol, or an alkane.

25. The system or method of embodiment 23, wherein said carbon-containing compound is one or more of 2-oxoglutarate, pyruvate, malate, fumarate, succinate, 4-hydroxybutyrate, 1,4 butanediol, glutaconic acid, 3-methyl-2-oxobutyrate, 3-methyl-2-oxovalerate, 4-methyl-2-oxopentanoate, isobutaraldehyde, isobutanol, 2-methyl-1-butanol, 3-methyl-2-butanol, isopentanol, glucose, glutathione, 3-phosphoglycerate, cis-aconitate, agmatine, putrescine, or glycyerin.

26. The system or method of any one of embodiments 1-25, wherein said photosynthetic microorganism comprises one or more introduced or overexpressed polynucleotides encoding one or more enzymes associated with lipid biosynthesis.

27. The system of method of embodiment 26, wherein said one or more enzymes associated with lipid biosynthesis comprises an acyl carrier protein (ACP), acyl ACP synthase (Aas), acyl-ACP reductase, alcohol dehydrogenase, aldehyde dehydrogenase, aldehyde decarbonylase, thioesterase (TES), acetyl coenzyme A carboxylase (ACCase), diacylglycerol acyltransferase (DGAT), phosphatidic acid phosphatase (PAP; or phosphatidate phosphatase), triacylglycerol (TAG) hydrolase, fatty acyl-CoA synthetase, lipase/phospholipase, or any combination thereof.

28. The system or method of embodiment 26, wherein said one or more enzymes comprises a diacylglycerol acyltransferase (DGAT), and wherein said carbon-containing compound comprises a triglyceride.

29. The system or method of embodiment 28, wherein said one or more enzymes further comprises a phosphatidate phosphatase, acetyl coenzyme A carboxylase (ACCase), acyl carrier protein (ACP), phospholipase B, phospholipase C, fatty acyl Co-A synthetase, or any combination thereof.

30. The system or method of embodiment 26, where said one or more enzymes comprises an acyl-ACP reductase.

31. The system or method of embodiment 30, wherein said one or more enzymes comprises a DGAT, and wherein said carbon-containing compound comprises a triglyceride.

32. The system or method of embodiment 31, wherein said one or more enzymes comprises an aldehyde dehydrogenase.

33. The system or method of embodiment 30, wherein said one or more enzymes comprises a DGAT having wax ester synthase activity and an alcohol dehydrogenase, and wherein said carbon-containing compound comprises a wax ester.

34. The system or method of embodiment 33, comprising reduced expression of an endogenous aldehyde dehydrogenase.

35. The system of method of any of embodiments 31-34, comprising reduced expression of an endogenous aldehyde decarbonylase.

36. The system or method of embodiment 30, wherein said one or more enzymes comprises an alcohol dehydrogenase, and wherein said carbon-containing compound comprises a fatty alcohol.

37. The system or method of embodiment 36, comprising reduced expression of an endogenous aldehyde decarbonylase, reduced expression of an endogenous aldehyde dehydrogenase, or both.

38. The system or method of embodiment 30, wherein said one or more enzymes comprises an aldehyde decarbonylase, and wherein said carbon-containing compound comprises an alkane.

39. The system or method of embodiment 38, comprising reduced expression of an endogenous aldehyde dehydrogenase, reduced expression of an endogenous alcohol dehydrogenase, or both.

40. The system or method of embodiment 30, wherein said carbon-containing compound is a fatty acid, optionally a free fatty acid.

41. The system or method of embodiment 40, wherein said one or more enzymes comprises an aldehyde dehydrogenase.

42. The system or method of embodiment 40 or 41, comprising reduced expression of an aldehyde decarbonylase, reduced expression of an endogenous alcohol dehydrogenase, or both.

43. A method for providing secretion of glucose from a photosynthetic microorganism, comprising culturing a modified photosynthetic microorganism in a media under a stress condition, wherein said photosynthetic microorganism:

(a) accumulates a reduced amount of glycogen as compared to the wild-type photosynthetic microorganism, and

(b) comprises one or more introduced or (over)expressed polynucleotides encoding a glucose permease,

wherein said modified photosynthetic microorganism maintains photosynthetic activity and accumulates reduced biomass when grown under said stress condition as compared to when grown under non-stress conditions.

44. A method of producing isobutanol or isopentanol, comprising culturing a modified photosynthetic microorganism in a media under a stress condition, wherein said photosynthetic microorganism:

(a) accumulates a reduced amount of glycogen as compared to the wild-type photosynthetic microorganism, and

(b) comprises one or more introduced or overexpressed polynucleotides encoding one or more polypeptides associated with production of isobutanol or isopentanol,

wherein said modified photosynthetic microorganism maintains photosynthetic activity and accumulates reduced biomass when grown under said stress condition as compared to when grown under non-stress conditions.

45. The method of embodiment 44, wherein said one or more polypeptides of (b) are a gene that converts a 2-keto acid to an aldehyde (2-keto acid decarboxylase), a gene that converts the aldehyde to an alcohol (alcohol dehydrogenase), or both.

46. A method of producing 4-hydroxybutyrate, comprising culturing a modified photosynthetic microorganism in a media under a stress condition, wherein said photosynthetic microorganism:

(a) accumulates a reduced amount of glycogen as compared to the wild-type photosynthetic microorganism, and

(b) comprises one or more introduced or overexpressed polynucleotides encoding one or more polypeptides associated with production of 4-hydroxybutyrate,

wherein said modified photosynthetic microorganism maintains photosynthetic activity and accumulates reduced biomass when grown under said stress condition as compared to when grown under non-stress conditions.

47. The method of embodiment 46, where said one or more polypeptides of (b) are an alpha ketoglutarate decarboxylase, a 4-hydroxybutyrate dehydrogenase, a succinyl-CoA synthetase, a succinate-semialdehyde dehydrogenase, or any combination thereof.

48. The method of embodiment 46 or 47, for producing 1,4-butanediol, wherein said photosynthetic microorganism: (c) further comprises one or more introduced or overexpressed polynucleotides encoding one or more polypeptides associated with production of 1,4-butanediol from 4-hydroxybutyrate.

49. The method of embodiment 48, wherein said one or more polypeptides of (c) are a 4-hydroxybutyryl-coA transferase, an aldehyde/alcohol dehydrogenase that is optionally capable of reducing coA-linked substrates to aldehydes/alcohols, or both.

50. A method of producing a polyamine intermediate, comprising culturing a modified photosynthetic microorganism in a media under a stress condition, wherein said photosynthetic microorganism:

(a) accumulates a reduced amount of glycogen as compared to the wild-type photosynthetic microorganism, and

(b) optionally comprises one or more introduced or overexpressed polynucleotides encoding one or more polypeptides associated with production of a polyamine intermediate,

wherein said modified photosynthetic microorganism maintains photosynthetic activity and accumulates reduced biomass when grown under said stress condition as compared to when grown under non-stress conditions.

51. The method of embodiment 50, where said polyamine intermediate is putrescine or agmatine.

52. The method of embodiment 50 or 51, wherein said one or more polypeptides is an arginine decarboxylase, and wherein said polyamine intermediate is agmatine.

53. The method of any of embodiments 50-52, wherein said one or more polypeptides is an arginine decarboxylase, an agmatine deiminase, or an N-carbamoylputrescine amidase, or any combination thereof, and wherein said polyamine intermediate is putrescine.

54. The method of any of embodiments 6-53, comprising relieving the stress condition when the ratio of absorbance of the culture at 680/750 nm is (or falls to) about 10%-90% of the ratio of a corresponding culture under non-stress conditions, where relieving the stress condition increases photosynthetic activity and/or the ratio of absorbance.

55. The method of embodiment 54, where the stress condition comprises reduced level of an essential nutrient, and relieving the stress condition comprises adding (pulsing the culture with) the essential nutrient in an amount sufficient to increase photosynthetic activity and/or the ratio of absorbance.

56. The method of embodiment 54 or 55, where said photosynthetic activity increases by at least about 10% relative to photosynthetic activity immediately prior to relief of said stress condition.

57. The method of any of embodiments 54-56, where the modified photosynthetic microorganism maintains the increased photosynthetic activity for a substantially longer time than a wild-type photosynthetic microorganism under the same or comparable culture conditions.

58. The method of any of embodiments 54-57, where the ratio of absorbance increases to greater than about 90% of the ratio of a corresponding culture under non-stress conditions, where non-stress conditions optionally comprise nutrient replete conditions.

59. The method of any of embodiments 54-58, where the modified photosynthetic microorganism culture maintains the increased ratio of absorbance for a substantially longer time than a wild-type photosynthetic microorganism culture under the same or comparable culture conditions.

60. The method of any of embodiments 54-57, where following relief of the stress condition and increased photosynthetic activity, the subsequent decrease in photosynthetic activity by the modified photosynthetic microorganism is substantially less than the subsequent decrease in photosynthetic activity by a wild-type photosynthetic microorganism culture under the same or comparable culture conditions.

61. The method of any of embodiments 54-60, further comprising repeating the step of relieving the stress condition when the ratio of absorbance falls to about 10%-90% of the ratio of a corresponding culture under non-stress conditions.

62. The method of any of embodiments 6-54, comprising relieving the stress condition at about 2, 3, 4, 5, 6, 7, 8, 9, 10, 11, 12, 13, or 14 days following initiation of the stress condition, where relieving the stress condition increases photosynthetic activity.

63. The method of embodiment 62, where the stress condition comprises reduced level of an essential nutrient, and relieving the stress condition comprises adding (pulsing the culture with) the essential nutrient in an amount sufficient to increase photosynthetic activity.

64. The method of embodiments 62 or 63, where said photosynthetic activity increases by at least about 10% relative to photosynthetic activity immediately prior to relief of said stress condition.

65. The method of any of embodiments 62-64, where the modified photosynthetic microorganism maintains the increased photosynthetic activity for a substantially longer time than a wild-type photosynthetic microorganism under the same or comparable culture conditions.

66. The method of any of embodiments 62-64, where following relief of the stress condition and increased photosynthetic activity, the subsequent decrease in photosynthetic activity by the modified photosynthetic microorganism is substantially less than the subsequent decrease in photosynthetic activity by a wild-type photosynthetic microorganism under the same or comparable culture conditions.

67. The method of any of embodiments 60-66, further comprising repeating the step of relieving the stress condition about every 2, 3, 4, 5, 6, 7, 8, 9, 10, 11, 12, 13, or 14 days following previous relief of the stress condition, where relieving the stress condition increases photosynthetic activity.

68. The method of any of embodiments 55-67, where the essential nutrient is selected from at least one of nitrogen, sulfur, and phosphorous.

69. The method of embodiment 68, where the essential nutrient is nitrogen.

70. The method of embodiment 69, where nitrogen is added in the form of NaNO₃, NH₄Cl, (NH₄)₂SO₄, NH₄HCO₃, CH₄N₂O, KNO₃, or any combination thereof, optionally to achieve a final concentration ranging from about 0.02 mM to about 20 mM.

71. The system or method of any of embodiments 1-70, where photosynthetic activity of the modified photosynthetic microorganism under the stress condition is least about 20% of photosynthetic activity of the modified photosynthetic microorganism or the wild-type photosynthetic microorganism under non-stress conditions.

72. The system or method of embodiment 71, where photosynthetic activity of the modified photosynthetic microorganism under the stress condition is least about 50% of photosynthetic activity of the modified photosynthetic microorganism or the wild-type photosynthetic microorganism under non-stress conditions.

73. The system or method of any of embodiments 1-72, where photosynthetic activity of the modified photosynthetic microorganism under the stress condition is substantially greater than photosynthetic activity of the wild-type photosynthetic microorganism under the stress condition.

74. The system or method of embodiment 73, where photosynthetic activity of the modified photosynthetic microorganism under the stress condition is at least about 1.5, 2, 3, 4, 5, 6, 7, 8, 9, 10 or 20-fold greater than photosynthetic activity of the wild-type photosynthetic microorganism under the stress condition.

75. The system or method of any of embodiments 71-74, where said photosynthetic activity is measured at about day 2, 3, 4, 5, 6, 7, 8, 9, 10, 11, 12, 13, or 14 post-initiation of the stress condition.

76. The system or method of any of embodiments 1-70, where maintenance of photosynthetic activity comprises maintenance of chlorophyll A levels.

77. The system or method of embodiment 76, where chlorophyll A levels of the modified photosynthetic microorganism under the stress condition are at least about 20% of chlorophyll A levels of the modified photosynthetic microorganism or the wild-type photosynthetic microorganism under non-stress conditions.

78. The system or method of embodiment 77, wherein chlorophyll A levels of the modified photosynthetic microorganism under the stress condition are at least about 50% of chlorophyll A levels of the modified photosynthetic microorganism or the wild-type photosynthetic microorganism under non-stress conditions.

79. The system or method of any of embodiments 76-78, where chlorophyll A levels of the modified photosynthetic microorganism under the stress condition are substantially greater than chlorophyll A levels of the wild-type photosynthetic microorganism under the stress condition.

80. The system or method of embodiment 79, where chlorophyll A levels of the modified photosynthetic microorganism under the stress condition are at least about 1.5, 2, 3, 4, 5, 6, 7, 8, 9, 10 or 20-fold greater than chlorophyll A levels of the wild-type photosynthetic microorganism under the stress condition.

81. The system or method of any of embodiments 76-80, where chlorophyll A levels are measured at about day 2, 3, 4, 5, 6, 7, 8, 9, 10, 11, 12, 13, or 14 post-initiation of the stress condition.

1.-63. (canceled)

64. A system for producing a carbon-containing compound, comprising:
a modified Cyanobacterium that accumulates a reduced amount of glycogen as compared to a corresponding wild-type Cyanobacterium; and
a culture system for culturing the modified Cyanobacterium under a stress condition,
wherein the modified Cyanobacterium maintains photosynthetic activity and accumulates reduced biomass when grown under the stress condition as compared to when grown under a non-stress condition.

65. The system of claim 64, wherein the modified Cyanobacterium secretes and/or intracellularly accumulates an increased amount of a carbon-containing compound when grown under the stress condition as compared to when grown under non-stress conditions, or as compared to the corresponding wild-type Cyanobacterium grown under the stress condition.

66. The system of claim 64, wherein the modified Cyanobacterium has reduced expression of one or more genes of a glycogen biosynthesis or storage pathway as compared to the corresponding wild-type photosynthetic microorganism, and wherein the one or more genes are selected from the group consisting of: a glucose-1-phosphate adenyltransferase (glgC) gene, a phosphoglucomutase (pgm) gene, and a glycogen synthase (glgA) gene.

67. The system of claim 64, wherein the stress condition is a reduced level of an essential nutrient, and wherein the essential 5 nutrient is selected from at least one of nitrogen, sulfur, and phosphorous.

68. The system of claim 64, wherein the modified Cyanobacterium further: comprises one or more introduced polynucleotides encoding a protein that increases glycogen breakdown; and/or comprises one or more introduced polynucleotides encoding a protein that increases secretion of a glycogen precursor.

69. The system of claim 64, wherein the Cyanobacterium is a Synechococcus elongatus.

70. The system of claim 64, wherein the carbon-containing compound is a lipid.

71. The system of claim 64, wherein the carbon-containing compound is one or more of 2-oxoglutarate, pyruvate, malate, fumarate, succinate, 4-hydroxybutyrate, 1,4 butanediol, glutaconic acid, 3-methyl-2-oxobutyrate, 3methyl-2-oxovalerate, 4-methyl-2-oxopentanoate, isobutaraldehyde, isobutanol, 2methyl-1-butanol, 3-methyl-2-butanol, isopentanol, glucose, glutathione, 3phosphoglycerate, cis-aconitate, agmatine, putrescine, or glycyerin.

72. The system of claim 64, wherein photosynthetic activity of the modified Cyanobacterium under the stress condition is substantially greater than photosynthetic activity of the corresponding wild-type Cyanobacterium under the stress condition.

73. The system of claim 64, wherein photosynthetic activity of the modified Cyanobacterium under the stress condition:
is at least about 1.5, 2, 3, 4, 5, 6, 7, 8, 9, 10 or 20-fold greater than photosynthetic activity of the corresponding wild-type Cyanobacterium under the stress condition; or
is measured at about day 2, 3, 4, 5, 6, 7, 8, 9, 10, 11, 12, 13, or 14 post-initiation of the stress condition.

74. A method for producing a carbon-containing compound other than glycogen, comprising culturing in a culture media under a stress condition a modified Cyanobacterium that accumulates a reduced amount of glycogen as compared to a corresponding wild-type Cyanobacterium, wherein the modified Cyanobacterium maintains photosynthetic activity and accumulates reduced biomass when grown under the stress condition as compared to when grown under non-stress conditions.

75. The method of claim 74, wherein the modified Cyanobacterium secretes and/or intracellularly accumulates an increased amount of a carbon-containing compound when grown under the stress condition as compared to when grown under non-stress conditions, or as compared to a corresponding wild-type Cyanobacterium grown under the stress condition.

76. The method of claim 74, wherein the modified Cyanobacterium has reduced expression of one or more genes of a glycogen biosynthesis or storage pathway as compared to the corresponding wild-type Cyanobacterium, and wherein the one or more genes are selected from the group consisting of: a glucose-1-phosphate adenyltransferase (glgC) gene, a phosphoglucomutase (pgm) gene, and a glycogen synthase (glgA) gene.

77. The system of claim 74, wherein the stress condition is a reduced level of an essential nutrient, and wherein the essential 5 nutrient is selected from at least one of nitrogen, sulfur, and phosphorous.

78. The method of claim 74, wherein the modified Cyanobacterium further: comprises one or more introduced polynucleotides encoding a protein that increases glycogen breakdown; and/or comprises one or more introduced polynucleotides encoding a protein that increases secretion of a glycogen precursor.

79. The method of claim 74, wherein Cyanobacterium is a Synechococcus elongatus.

80. The method of claim 74, wherein the carbon-containing compound is a lipid.

81. The method of claim 74, wherein the carbon-containing compound is one or more of 2-oxoglutarate, pyruvate, malate, fumarate, succinate, 4-hydroxybutyrate, 1,4 butanediol, glutaconic acid, 3-methyl-2-oxobutyrate, 3-methyl-2-oxovalerate, 4-methyl-2-oxopentanoate, isobutaraldehyde, isobutanol, 2-methyl-1-butanol, 3-methyl-2-butanol, isopentanol, glucose, glutathione, 3-phosphoglycerate, cis-aconitate, agmatine, putrescine, or glycyerin.

82. The method of claim 74, wherein photosynthetic activity of the modified Cyanobacterium under the stress condition is substantially greater than photosynthetic activity of the corresponding wild-type Cyanobacterium under the stress condition.

83. The method of claim 74, wherein the maintained photosynthetic activity comprises maintenance of chlorophyll A levels, wherein the chlorophyll A levels:
are at least about 1.5, 2, 3, 4, 5, 6, 7, 8, 9, 10 or 20-fold greater than chlorophyll A levels of the 10 corresponding wild-type Cyanobacterium under the stress condition.
are measured at about day 2, 3, 4, 5, 6, 7, 8, 9, 10, 11, 12, 13, or 14 post-initiation of the stress condition.
